(19) United States
(12) Patent Application Publication
(10) Pub. No.: US 2013/0266554 A1
(43) Pub. Date: Oct. 10, 2013

(54) Lysozymes

(71) Applicants: Mikkel Klausen, Copenhagen (DK); Marie Allesen Holm, Hilleroed (DK); Leonardo De Maria, Frederiksberg (DK); Michael Skjoet, Roskilde (DK)

(72) Inventors: Mikkel Klausen, Copenhagen (DK); Marie Allesen Holm, Hilleroed (DK); Leonardo De Maria, Frederiksberg (DK); Michael Skjoet, Roskilde (DK)

(73) Assignee: NOVOZYMES A/S, Bagsvaerd (DK)

(21) Appl. No.: 13/819,918

(22) PCT Filed: Sep. 15, 2011
(86) PCT No.: PCT/EP11/66015

§ 371 Date: Feb. 28, 2013

(30) Foreign Application Priority Data
Sep. 16, 2010 (EP) 10177020.4

(51) Int. Cl. C12N 9/24

(52) U.S. Cl. CPC C12N 9/2462
USPC 424/94.61; 435/206; 536/23.2

(57) ABSTRACT

The present invention is directed to lysozyme variants and nucleotide sequences encoding same. The lysozyme variants have antimicrobial and/or lysozyme activity and comprise an alteration of an amino acid sequence at one or more positions. The present invention is also directed to methods for producing and of using the Opisthocomus hoazin lysozyme and the lysozyme variants of the present invention as antimicrobial agents.

### REFERENCE TO A SEQUENCE LISTING

This application contains a Sequence Listing in computer readable form. The computer readable form is incorporated herein by reference.

### REFERENCE TO ATOMIC COORDINATES

This application sets forth in FIG. 1, the atomic coordinates of the three-dimensional structure of the Opisthocomus hoazin lysozyme.

### FIELD OF THE INVENTION

The present invention relates to lysozymes and their use, including as anti-microbial agents.

### BACKGROUND OF THE INVENTION

Lysozyme (EC 3.2.1.17), also known as muramidase or N-acetylmuramide glycanhydrolase, catalyzes hydrolysis of 1,4-beta-linkages between N-acetylmuramic acid and N-acetyl-D-glucosamine residues in a peptidoglycan and between N-acetyl-D-glucosamine residues in chitodextrins. Lysozymes are typically produced as a defensive mechanism against bacteria by many organisms, including viruses, plants, insects, birds, reptiles and mammals. Lysozymes hydrolyze bacterial cell walls by cleaving the glycosidic bonds of peptidoglycan, an important structural molecule in bacteria. After having their cell walls weakened by lysozyme action, bacterial cells lyse resulting from osmotic pressure. There is an increasing interest in the potential of lysozymes as anti-microbial agents. For example, lysozyme activity has been shown against pathogens such as Streptococcus pneumoniae, Bacillus anthracis, Enterococcus faecium, Bacillus stearothermophilus, Clostridium botulinum, Clostridium butyricum, Clostridium perfringens, Clostridium sporogenes, Clostridium tyrobutyricum, and Listeria monocytogenes.

Lysozymes have been classified into five different glycoside hydrolase (GH) families (CAZy, www.cazy.org): hen egg-white lysozyme (GH22), goose egg-white lysozyme (GH23), bacteriophage T4 lysozyme (GH24), Sphingomonas flagellar protein (GH73) and Chalaropsis lysozymes (GH25).

### RELATED PRIOR ART

As indicated above lysozymes have been shown to be active against a number of pathogens, see (Zhang G, Darius S, Smith S R, Ritchie S J. In vitro inhibitory effect of hen egg white lysozyme on Clostridium perfringens type A associated with broiler necrotic enteritis and its alpha-toxin production. Lett Appl Microbiol. 2006 February; 42(2):138-43. Cunningham F E, Proctor V A, Goetsch S J. Egg-white lysozyme as a food preservative: an overview. World's Poultry Science Journal. 1991; 47: 141-163).

Lysozymes have been disclosed for use in animal feed (WO 00/21381 and WO 04/026334), cheese production (WO 05/080559), food preservation (Hughey and Johnson (1987) Appl Environ Microbiol 53:2165), detergents (U.S. Pat. No. 5,041,236 and EP 0425016), oral care (U.S. Pat. No. 4,355,022, WO 04/017988 and WO 08/124,764), cosmetology and dermatology, contraception, urology, and gynecology (WO 08/124,764).

Hen egg-white lysozyme is a commercially available lysozyme product. Lysozymes isolated from other animal (vertebrate, birds and mammals), viral and microbial sources are also known.

Hoatzin (Opisthocomus hoazin), is an unusual species of tropical bird found in swamps, riverine forest and mangrove of the Amazon and the Orinoco delta in South America. It is the only member of the genus Opisthocomus.

The amino acid sequence, genomic DNA and cDNA sequences of the Hoatzin lysozyme were disclosed by Kornegay et al. Mol. Biol. Evol. 11 (6):921-928. 1994,

In an article, Wada et al. “A molecular neuroethological approach for identifying and characterizing a cascade of behaviorally regulated genes.” Proc Natl Acad Sci USA, 103:15212-15217 (2006) an amino acid sequence having an identity of 77.8% to the Hoatzin lysozyme sequence has been disclosed.

### SUMMARY OF THE INVENTION

The present invention is directed to lysozyme variants and nucleotide sequences encoding same. The lysozyme variants have antimicrobial and/or lysozyme activity and comprise an alteration/modification of an amino acid sequence at one or more positions selected from the group consisting of position number 3, 9, 10, 11, 17, 20, 25, 29, 32, 34, 37, 41, 47, 50, 60, 61, 67, 69, 73, 74, 81, 87, 88, 89, 90, 91, 92, 96, 97, 98, 101, 104, 108, 109, 111, and/or 124, which position corresponds to a position in amino acid sequence SEQ ID NO:5 of the mature protein.

The present invention is also directed to methods of using the hoazin (Opisthocomus hoazin) lysozyme and lysozyme variants as antimicrobial agents. In particular embodiments, the present invention relates to the use of the Opisthocomus hoazin lysozyme and lysozymes variants of the present invention as antimicrobial agents in animal feed, cheese production, food preservation, detergents, oral care, cosmetology and dermatology.

In another embodiment, the present invention also relates to the use of the Opisthocomus hoazin lysozyme and the lysozyme variants of the present invention as antimicrobial agents in a method of producing a fermentation product, such as, ethanol from biomass. The lysozymes may be used to reduce unwanted microbial growth in a fermentation process or other process steps susceptible to microbial contamination.

The hoazin lysozyme exhibits high stability at low pH (Kornegay et al. Mol. Biol. Evol. 11 (6):921-928. 1994) that potentially could target Clostridium perfringens,—the bacteria responsible for the disease clostridial necrotic enteritis in poultry. This disease may appear as “clinical” with increased mortality or “subclinical” with poor growth and feed conversion, both of which have severe economic implication for poultry farmers (Craven et al., 2003).

The present invention is also directed to compositions comprising the Opisthocomus hoazin lysozyme and the lysozyme variants of the present invention, such as, cleaning or disinfecting compositions.

The present invention is also directed to methods for producing the Opisthocomus hoazin lysozyme and the lysozyme variants of the present invention recombinantly in microbial host cells, especially bacteria and fungi.

### BRIEF DESCRIPTION OF THE FIGURES AND SEQUENCE LISTING

FIG. 1 sets forth the atomic coordinates of the three-dimensional structure of the Opisthocomus hoazin lysozyme. These atomic coordinates can aid in generating a three dimensional model depicting the structure of the Opisthocomus hoazin lysozyme and a three dimensional model of homologous structures, such as, the variants of the present invention.

In the Sequence Listing, the sequences apply as follows:

| | |
|---|---|
| SEQ ID NO: 1 | Opisthocomus hoazin lysozyme nucleic acid sequence |
| SEQ ID NO: 2 | Opisthocomus hoazin lysozyme predicted from SEQ ID |
| | NO: 1 |
| SEQ ID NO: 3 | optimized Opisthocomus hoazin lysozyme nucleic acid |
| | sequence |
| SEQ ID NO: 4 | Opisthocomus hoazin lysozyme from SEQ ID NO: 3 |
| SEQ ID NO: 5 | Opisthocomus hoazin lysozyme, mature protein |
| SEQ ID NO: 6 | KexB mature Opisthocomus hoazin lysozyme |

### DETAILED DESCRIPTION OF THE INVENTION

### Antimicrobial Activity:

The term “antimicrobial activity” is defined herein as is an activity that kills or inhibits the growth of microorganisms, such as, algae, archea, bacteria, fungi and/or protozoans. The antimicrobial activity can for example be bactericidal meaning the killing of bacteria or bacteriostatic meaning the prevention of bacterial growth. The antimicrobial activity can include catalyzing the hydrolysis of 1,4-beta-linkages between N-acetylmuramic acid and N-acetyl-D-glucosamine residues in a peptidoglycan and between N-acetyl-D-glucosamine residues in chitodextrins. Antimicrobial activity can also include the lysozyme binding to the surface of the microorganism and inhibiting its growth. The antimicrobial effect can also include the use of the lysozymes of the present invention for activation of bacterial autolysins, as an immunostimulator, by inhibiting or reducing bacterial toxins and by an opsonin effect. For purposes of the present invention, antimicrobial activity is determined according to the lysozyme turbidity activity assay described in the “Materials and Methods” section.

### Lysozyme Activity:

The term “lysozyme activity” is defined herein as a peptidoglycan N-acetylmuramoylhydrolase activity (EC 3.2.1.17) that catalyzes the hydrolysis of 1,4-beta-linkages between N-acetylmuramic acid and N-acetyl-D-glucosamine residues in a peptidoglycan and between N-acetyl-D-glucosamine residues in chitodextrins. For purposes of the present invention, lysozyme activity is determined according to the lysozyme turbidity activity assay described in the “Materials and Methods” section.

### Variant:

The term “variant” is defined herein as a polypeptide having antimicrobial activity and/or lysozyme activity comprising an alteration, such as a substitution, insertion, and/or deletion, of one or more (several) amino acid residues at one or more (several) positions. The variants of the present invention have at least one alteration/modification selected from the group consisting of position number 3, 9, 10, 11, 17, 20, 25, 29, 32, 34, 37, 41, 47, 50, 60, 61, 67, 69, 73, 74, 81, 87, 88, 89, 90, 91, 92, 96, 97, 98, 101, 104, 108, 109, 111, and 124, which position corresponds to a position in amino acid sequence SEQ ID NO:5 of the mature protein. and/or deletion, of one or more (several) amino acid residues at one or more (several) positions. The variants of the present invention have at least one alteration/modification selected from the group consisting of position number 3, 9, 10, 11, 17, 20, 25, 29, 32, 34, 37, 41, 47, 50, 60, 61, 67, 69, 73, 74, 81, 87, 88, 89, 90, 91, 92, 96, 97, 98, 101, 104, 108, 109, 111, and 124, The variant polypeptide sequence is preferably one which is not found in nature.

### Wild-Type Lysozyme:

The term “wild-type” lysozyme denotes a lysozyme expressed by a naturally occurring organism, preferably from a naturally occurring microorganism, such as, algae, archea, bacteria, yeast, filamentous fungus, protozoan, insect, fish, plant, and animal (vertebrate, such as avian, and mammalian) comprising the gene encoding the lysozyme in the natural genome as found in nature. The term wild-type may be used interchangeably with the term “naturally occurring”.

### Parent Enzyme:

The term “parent” lysozyme or “parental” lysozyme as used herein means a lysozyme to which a modification, e.g. substitution(s), insertion(s), deletion(s), and/or truncation(s), is made to produce the enzyme variants of the present invention. Alternatively or in addition, this term also refers to the polypeptide with which a variant of the present invention is compared and/or aligned. The parent may be a naturally occurring (wild-type) polypeptide, such as, the Opisthocomus hoazin lysozyme, the lysozyme encoded by the nucleic acid sequence of SEQ ID NO:1 or SEQ ID NO: 3, the mature lysozyme of SEQ ID NO:5 or SEQ ID NO:6, or a polypeptide which is at least 80%, identical to such parent lysozymes. The parent polypeptide may also be a variant of a naturally occurring polypeptide which has been modified or altered in the amino acid sequence. A parent may also be an allelic variant, which is a polypeptide encoded by any of two or more alternative forms of a gene occupying the same chromosomal locus.

The parent may be a naturally occurring (wild-type) polypeptide, such as, the Opisthocomus hoazin lysozyme, the lysozyme encoded by the nucleic acid sequence of SEQ ID NO:1 or SEQ ID NO: 3, the mature lysozyme of SEQ ID NO:5 or SEQ ID NO:6, or a polypeptide which is at least 85%, identical to such parent lysozymes. The parent polypeptide may also be a variant of a naturally occurring polypeptide which has been modified or altered in the amino acid sequence. A parent may also be an allelic variant, which is a polypeptide encoded by any of two or more alternative forms of a gene occupying the same chromosomal locus.

The parent may be a naturally occurring (wild-type) polypeptide, such as, the Opisthocomus hoazin lysozyme, the lysozyme encoded by the nucleic acid sequence of SEQ ID NO:1 or SEQ ID NO: 3, the mature lysozyme of SEQ ID NO:5 or SEQ ID NO:6, or a polypeptide which is at least 90%, identical to such parent lysozymes. The parent polypeptide may also be a variant of a naturally occurring polypeptide which has been modified or altered in the amino acid sequence. A parent may also be an allelic variant, which is a polypeptide encoded by any of two or more alternative forms of a gene occupying the same chromosomal locus.

The parent may be a naturally occurring (wild-type) polypeptide, such as, the Opisthocomus hoazin lysozyme, the lysozyme encoded by the nucleic acid sequence of SEQ ID NO:1 or SEQ ID NO: 3, the mature lysozyme of SEQ ID NO:5 or SEQ ID NO:6, or a polypeptide which is at least 95%, identical to such parent lysozymes. The parent polypeptide may also be a variant of a naturally occurring polypeptide which has been modified or altered in the amino acid sequence. A parent may also be an allelic variant, which is a polypeptide encoded by any of two or more alternative forms of a gene occupying the same chromosomal locus.

The parent may be a naturally occurring (wild-type) polypeptide, such as, the Opisthocomus hoazin lysozyme, the lysozyme encoded by the nucleic acid sequence of SEQ ID NO:1 or SEQ ID NO: 3, the mature lysozyme of SEQ ID NO:5 or SEQ ID NO:6, or a polypeptide which is at least 96%, at least 97%, at least 98% or at least 99% identical to such parent lysozymes. The parent polypeptide may also be a variant of a naturally occurring polypeptide which has been modified or altered in the amino acid sequence. A parent may also be an allelic variant, which is a polypeptide encoded by any of two or more alternative forms of a gene occupying the same chromosomal locus.

### Isolated Variant or Polypeptide:

The term “isolated variant” or “isolated polypeptide” as used herein refers to a variant or a polypeptide that is isolated from a source, e.g., the host cell from which it is expressed or the enzyme complex it is normally present in. Preferably, the polypeptide is at least 40% pure, such as, at least 60% pure, at least 80% pure, at least 90% pure, at least 95% pure, or 100% pure as determined by SDS-PAGE.

### Substantially Pure Variant or Polypeptide:

The term “substantially pure variant” or “substantially pure polypeptide” denotes herein a polypeptide preparation that contains at most 10%, such as, at most 8%, at most 6%, at most 5%, at most 4%, at most 3%, at most 2%, at most 1%, or at most 0.5% by weight of other polypeptide material with which it is natively or recombinantly associated. It is, therefore, preferred that the lysozyme variant is at least 92% pure, such as, at least 94% pure, at least 95% pure, at least 96% pure, at least 96% pure, more at least 97% pure, at least 98% pure, at least 99%, at least 99.5% pure or even 100% pure by weight of the total polypeptide material present in the preparation. The variants and polypeptides of the present invention are preferably in a substantially pure form. This can be accomplished, for example, by preparing the variant or polypeptide by well-known recombinant methods or by classical purification methods.

### Mature Polypeptide:

The term “mature polypeptide” is defined herein as a polypeptide having antimicrobial and/or lysozyme activity that is in its final form following translation and any post-translational modifications, such as N-terminal processing, C-terminal truncation, glycosylation, phosphorylation, etc. For the polypeptide defined by SEQ ID NO:2, an example of a mature lysozyme sequence starts at 19^{th} amino acid residue of SEQ ID NO:2 and ends at the 145^{th} amino acid residue of SEQ ID NO:2, which is the polypeptide shown in SEQ ID NO:3 and 5. Depending on expression system, however, the length of the actual mature polypeptide may vary, such as, e.g. 1 to 10 amino acids in length (longer or shorter) at the N and/or C termini from the predicted mature polypeptide.

### Mature Polypeptide Coding Sequence:

The term “mature polypeptide coding sequence” is defined herein as a nucleotide sequence that encodes a mature polypeptide having antimicrobial and/or lysozyme activity. In one aspect, the mature polypeptide coding sequence are nucleotides 58 to 436 of SEQ ID NO:1 or nucleotides 1 to 378 of SEQ ID NO:3. The mature polypeptide coding sequence may vary 3 to 30 nucleotides in length depending on the expression system.

### Identity:

The parameter “identity” as used herein describes the relatedness between two amino acid sequences or between two nucleotide sequences. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.orq), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al. (2000) supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

### Homologous Sequence:

The term “homologous sequence” is defined herein as a predicted polypeptide that gives an E value (or expectancy score) of less than 0.001 in a tfasty search (Pearson, W. R., 1999, Bioinformatics Methods and Protocols, S. Misener and S. A. Krawetz, ed., pp. 185-219) with the Opisthocomus hoazin lysozyme.

### Functional Fragment of a Polypeptide:

The term “functional fragment of a polypeptide” is used to describe a polypeptide which is derived from a longer polypeptide, e.g., a mature polypeptide, and which has been truncated either in the N-terminal region or the C-terminal region or in both regions to generate a fragment of the parent polypeptide. To be a functional polypeptide, the fragment must maintain antimicrobial and/or lysozyme activity, such as, maintain at least 20% of the full-length/mature polypeptide antimicrobial and/or lysozyme activity, such as, at least 40%, at least 50%, at least 60%, at least 70%, at least 80%, at least 90%, at least 95% of the antimicrobial and/or lysozyme activity of the full-length/mature polypeptide.

To be a functional polypeptide, the fragment must maintain antimicrobial and/or lysozyme activity, such as, maintain at least 50% of the full-length/mature polypeptide antimicrobial and/or lysozyme activity.

To be a functional polypeptide, the fragment must maintain antimicrobial and/or lysozyme activity, such as, maintain at least 60% of the full-length/mature polypeptide antimicrobial and/or lysozyme activity.

To be a functional polypeptide, the fragment must maintain antimicrobial and/or lysozyme activity, such as, maintain at least 80% of the full-length/mature polypeptide antimicrobial and/or lysozyme activity.

### Allelic Variant:

The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

### Isolated Polynucleotide:

The term “isolated polynucleotide” as used herein refers to a polynucleotide that is isolated from a source. In one aspect, the isolated polynucleotide is at least 40% pure, such as, at least 60% pure, at least 80% pure, at least 90% pure, at least 95% pure, or 100% pure, as determined by agarose electrophoresis.

### Substantially Pure Polynucleotide:

The term “substantially pure polynucleotide” as used herein refers to a polynucleotide preparation free of other extraneous or unwanted nucleotides and in a form suitable for use within genetically engineered polypeptide production systems. Thus, a substantially pure polynucleotide contains at most 10%, such as, at most 8%, at most 6%, at most 5%, at most 4%, at most 3%, at most 2%, at most 1%, or at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the polynucleotide is at least 90% pure, such as, at least 92% pure, at least 94% pure, at least 95% pure, at least 96% pure, at least 97% pure, at least 98% pure, at least 99%, or even at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semi-synthetic, synthetic origin, or any combinations thereof.

### Coding Sequence:

When used herein, the term “coding sequence” means a polynucleotide, which directly specifies the amino acid sequence of its polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

### Operably Linked:

The term “operably linked” denotes herein a configuration in which a control sequence is placed at an appropriate position relative to the coding sequence of the polynucleotide sequence such that the control sequence directs the expression of the coding sequence of a polypeptide.

### Host Cell:

The term “host cell”, as used herein, includes any cell type that is susceptible to transformation, transfection, transduction, and the like with a nucleic acid construct or a vector comprising a polynucleotide of the present invention. The term “host cell” encompasses any progeny of a parent cell that is not identical to the parent cell due to mutations that occur during replication.

### Altered/Modified Property:

The term “altered/modified property” is defined herein as a characteristic associated with a variant that is altered, as compared relative to the parent lysozyme or an identified reference sequence. Such altered properties include, but are not limited to, altered substrate specificity, altered pH-dependent activity profile, altered pH stability, altered pH optimum/activity, altered temperature stability, and/or altered resistance to non-enzymatic glycation.

### Improved Property:

The term “improved property” is defined herein as a characteristic associated with a variant that is improved, unless otherwise stated, relative to another reference lysozyme or the parent lysozyme. Such improved properties include, but are not limited to modified or improved substrate specificity, improved stability at alkaline or acidic pH conditions, a broader pH activity profile, and/or increased resistance to glycation. Improved properties may also include thermal properties, such as thermostability, steam stability, broader temperature activity profile. Further improved properties may include pelleting stability, protease-sensibility, and/or glycosylation pattern. Improvements are preferably assessed in relation to the desired application conditions.

### Substrate Specificity:

The term “substrate specificity” refers to the specificity of the lysozyme in regard to the type of bacteria it can kill and/or in relation to model lysozyme substrates (p-NP-(NAG-NAM)n or p-NP-(NAG)m oligomers).

### Thermostability:

The term “thermostability” refers to the lysozyme activity after a period of incubation at elevated temperature relative to the parent or an identified reference sequence, either in a buffer or under conditions such as those which exist during product storage/transport or conditions similar to those that exist during industrial use of the variant. A variant may or may not display an altered thermal activity profile relative to the parent. For example, a variant may have an improved ability to refold following incubation at an elevated temperature relative to the parent, e.g., at a temperature in the range of 45° C. to 110° C. In one aspect, the thermostability of the variant having lysozyme activity is at least 1.0-fold, e.g., at least 1.1-fold, at least 1.5-fold, at least 1.8-fold, at least 2-fold, at least 5-fold, at least 10-fold, at least 15-fold, at least 20-fold, and at least 25-fold more thermostable than the parent or reference sequence at the selected temperature. Preferably the activity is tested using the lysozyme turbidity activity assay described in the “Materials and Methods” section, with the deviation of the temperature to the desired increased temperature.

In one aspect, the thermostability of a lysozyme variant is improved such that the variant can survive high temperatures, e.g. temperatures from 45° C. to 110° C., preferably from 50° C. to 100° C., more preferably from 60° C. to 90° C., even more preferably from 70° C. to 80° C. Preferably, the variant lysozyme maintains at least 40%, preferably at least 50%, 60%, 70% or 80%, more preferably at least 90%, even more preferably at least 95% residual activity after incubation at a given high temperature for 1 hour when compared to the variant which has been maintained at room temperature for the same time. Preferably, the residual activity of the variant lysozyme is at least 1.5-fold, preferably at least 2-fold, more preferably at least 5-fold, most preferably at least 7-fold, and even most preferably at least 20-fold higher than the residual activity of the parent lysozyme which has been treated under the same conditions. Preferably the activity is tested using the lysozyme turbidity activity assay described in the “Materials and Methods” section, with the deviation of the temperature to the desired increased temperature.

### Heat-Stability:

Heat stability may be determined as described in Example 1 by determining the temperature/activity profile of the variant lysozyme.

### Temperature Profile/Temperature Stability:

Whether or not a lysozyme of the invention has a modified temperature profile as compared to a reference lysozyme may be determined as described in Example 1. Accordingly, in a particular embodiment the lysozyme of the invention has a modified temperature profile as compared to a reference lysozyme, wherein the temperature profile is determined as lysozyme activity as a function of temperature. The activity at each temperature is preferably indicated as relative activity (in %) normalized to the value at optimum temperature. The optimum temperature is that temperature within the tested temperatures (i.e. those with 5-10° C. jumps) where the activity is highest.

### pH Stability:

The term “pH stability” refers to the variant enzyme displaying structural stability relative to the parent enzyme after a period of incubation at a pH which is outside the pH range where the enzyme is active (pH activity range). Such a variant may or may not display an altered pH activity profile relative to the parent. For example, the variant may not be active at the increased or decreased pH, but is able to maintain its three dimensional structure and then regain activity once it is returned to the pH activity range. Alternatively, the variant may have an improved ability to refold relative to the parent following incubation at increased or decreased pH.

In one aspect, the pH stability profile is altered such that a lysozyme variant has improved pH stability at alkaline pH. As used herein, alkaline pH means from pH 7.5 to 12, including from 8 to 11, more preferably from 8.5 to 10, even more preferably from 9 to 9.5. Preferably, the variant lysozyme maintains at least 40%, preferably at least 50%, 60%, 70% or 80%, more preferably at least 90%, even more preferably at least 95% residual activity after incubation at a given pH for 1 hour when compared to the variant which has been maintained at pH 6.5 for the same time. Preferably, the residual activity of the variant lysozyme is at least 1.5-fold, preferably at least 2-fold, more preferably at least 5-fold, most preferably at least 7-fold, and even most preferably at least 20-fold higher than the residual activity of the parent lysozyme which has been treated under the same conditions. Preferably, the activity is tested using the lysozyme turbidity activity assay described in the “Materials and Methods” section, with the deviation of the pH to the desired increased pH. A lysozyme variant with improved stability at alkaline pH (e.g. from pH 7.5 to 12, preferably from 8 to 11, more preferably from 8.5 to 10, even more preferably from 9 to 9.5) will be able to function in more alkaline environments such as detergents.

In one aspect, the pH stability profile is altered such that a lysozyme variant has improved stability at acidic pH. As used herein, acidic pH means from pH 2 to 5.5, preferably from 2.5 to 5.25, more preferably from 3 to 5, even more preferably from 3.5 to 4. Preferably, the variant lysozyme maintains at least 40%, preferably at least 50%, 60%, 70% or 80%, more preferably at least 90%, even more preferably at least 95% residual activity after incubation at a given pH for 1 hour when compared to the variant which has been maintained at pH 6.5 for the same time. Preferably, the residual activity of the variant lysozyme is at least 1.5-fold, preferably at least 2-fold, more preferably at least 5-fold, most preferably at least 7-fold, and even most preferably at least 20-fold higher than the residual activity of the parent lysozyme which has been treated under the same conditions. Preferably, the activity is tested using the lysozyme turbidity activity assay described in the “Materials and Methods” section, with the deviation of the pH to the desired decreased pH. A lysozyme variant with improved stability at acidic pH (e.g. from pH 2 to 5.5, preferably from 2.5 to 5.25, more preferably from 3 to 5, even more preferably from 3.5 to 4) will be able to function under more acidic conditions, such as preservative in certain foods or as a eubiotic molecule in feeds.

### Improved pH Activity:

The term “improved pH activity” is defined herein as a variant enzyme displaying an alteration of the pH-dependent activity profile when compared to the pH activity profile of the parent lysozyme or reference lysozyme. The pH activity profile provides a measure of the enzyme's efficiency in preventing microbial growth, eliminating microbial cells and/or performing catalysis of a hydrolysis reaction over a pH range at given conditions such as temperature and solvent composition. A lysozyme has a specific pH range wherein the polypeptide is stable and retains its enzymatic activity, outside this range the lysozyme becomes less active and potentially also less stable. Within the pH range there generally is a pH optimum, where the lysozyme shows the highest activity.

A lysozyme variant with improved activity at alkaline pH (e.g. from pH 7.5 to 12, preferably from 8 to 11, more preferably from 8.5 to 10, even more preferably from 9 to 9.5) will be able to function in more alkaline environments such as detergents.

A variant with improved activity at acidic pH (e.g. from pH 2 to 5.5, preferably from 2.5 to 5.25, more preferably from 3 to 5, even more preferably from 3.5 to 4) will be able to function under more acidic conditions, such as preservative in certain foods or as a eubiotic molecule in feeds.

In one aspect, the pH activity profile is altered such that a lysozyme variant has improved activity at alkaline pH. Preferably, the activity of the lysozyme variant is compared with the activity of the parent lysozyme or reference lysozyme at a pH at least 0.5 units above the optimum pH of the parent enzyme, preferably 1, 1.5, 2, 2.5 or 3 pH units above the optimum pH of the parent enzyme or reference lysozyme, most preferably at least 3.5 pH units above the optimum pH of the parent enzyme or reference lysozyme and most preferably at least 4 pH units above the optimum pH of the parent enzyme or reference lysozyme, under which conditions the variant has an activity which is at least 1.5-fold, preferably at least 2-fold, more preferably at least 5-fold, most preferably at least 7-fold and even most preferably at least 20-fold higher than that of the parent enzyme or reference lysozyme. Preferably, the lysozyme variant at the same time maintains at least 40%, preferably at least 50%, 60%, 70% or 80%, or 90%, more preferably at least 95%, even more preferably at least 100% of the activity that parent lysozyme or reference lysozyme exhibits at its pH optimum. Preferably, the activity is tested using the lysozyme turbidity activity assay described in the “Materials and Methods” section, with the deviation of the pH to the desired increased pH.

In one aspect, the pH activity profile is altered such that a lysozyme variant has improved activity at acidic pH. Preferably, the activity of the lysozyme variant is compared with the activity of the parent lysozyme or reference lysozyme at a pH at least 0.5 units below the optimum pH of the parent enzyme, preferably 1, 1.5, 2, 2.5 or 3 pH units below the optimum pH of the parent enzyme or reference lysozyme, most preferably at least 3.5 pH units below the optimum pH of the parent enzyme or reference lysozyme and most preferably at least 4 pH units below the optimum pH of the parent enzyme or reference lysozyme, under which conditions the variant has an activity which is at least 1.5-fold, preferably at least 2-fold, more preferably at least 5-fold, most preferably at least 7-fold and even most preferably at least 20-fold higher than that of the parent enzyme. Preferably, the lysozyme variant at the same time maintains at least 40%, preferably at least 50%, 60%, 70% or 80%, or 90%, more preferably at least 95%, even more preferably at least 100% of the activity that parent lysozyme or reference lysozyme exhibits at its pH optimum. Preferably the activity is tested using the lysozyme turbidity activity assay described in the “Materials and Methods” section, with the deviation of the pH to the desired decreased pH.

### Glycation Susceptibility

Non-enzymatic glycation is a spontaneous posttranslational process where reducing sugars bind covalently to free amino groups in proteins primarily at Lysine (K) residues. Glycation may impact the activity of the lysozyme. In accordance with the present invention, the susceptibility of the lysozyme to non-enzymatic glycation may be reduced by specified amino acid changes. The effect of glycation, and improvement relative to a parent or reference enzyme, may be determined as described in the “Materials and Methods” section.

### Conventions for Designation of Variants

For purposes of the present invention, the amino acid sequence of the lysozyme disclosed in SEQ ID NO:5 is used to determine the corresponding amino acid residue in another lysozyme. The amino acid sequence of another lysozyme is aligned with the amino acid sequence of the lysozyme disclosed in SEQ ID NO:5, and based on the alignment the amino acid position number corresponding to any amino acid residue in the amino acid sequence of the lysozyme disclosed in SEQ ID NO:5 can be determined.

An alignment of polypeptide sequences may be made using the Needleman-Wunsch algorithm (Needleman and Wunsch (1970) J. Mol. Biol. 48:443-453) as implemented in the Needle program of the EMBOSS package (EMBOSS: The European Molecular Biology Open Software Suite, Rice et al. (2000) Trends in Genetics 16:276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix.

In describing the various lysozyme variants of the present invention, the nomenclature described below is adapted for ease of reference. In all cases, the accepted IUPAC single letter or triple letter amino acid abbreviation is employed.

The alteration(s) are an insertion and/or deletion of the amino acid which occupies the position, and/or a substitution of the amino acid which occupies the position with a different amino acid.

### Substitutions.

For an amino acid substitution, the following nomenclature is used: original amino acid/position/substituted amino acid. Accordingly, the substitution of threonine with alanine at position 226 is designated as “Thr226Ala” or “T226A”. Multiple substitutions are separated by addition marks (“+”), e.g., “G205R+5411F”, representing substitutions at positions 205 and 411 substituting glycine (G) with arginine (R), and serine (S) with phenylalanine (F), respectively. Where an original amino acid may be substituted by an amino acid selected from a group it is designated as “K129R,S,A,I,F,Q” representing the substitution of a lysine (K) at position 129 with an amino acid selected from the group consisting of: arginine (R), serine (S), alanine (A), isoleucine (I), phenylalanine (F) and glutamine (Q). Alternatively, “K129R,S,A,I,F,Q” could be written as K129R or K129S, or K129A, or K129I or K129F or K129Q. Where an original amino acid may be substituted by any amino acid it is designated as “K129X”

### Deletions.

For an amino acid deletion, the following nomenclature is used: original amino acid/position/asterisk (*). Accordingly, the deletion of glycine at position 195 is designated as “Gly195*” or “G195*”. Multiple deletions are separated by addition marks (“+”), e.g. G195*+S411*.

For both substitutions and deletions the designation 195G/X195G or 195*/X195* is used to show that independent of the amino acid in the indicated position of the parent/starting protein, the modification has to be to G or a deletion, respectively.

### Insertions.

For an amino acid insertion, the following nomenclature is used: asterisk (*)/position/lower case letter/inserted amino acid, where the lower case letter indicates the addition of an amino acid downstream of the position number. Accordingly, the insertion of a glutamic acid (E) downstream of position 10 is designated “*10aE”. If a second amino acid, e.g. a valine (V), is to be inserted downstream of position 10 after the glutamic acid (E) it is designated “*10aE+*10bV”. Additions to the N-terminal of the polypeptide are designated with a 0 (zero). The addition of a glutamic acid (E) and a valine (V) added to the N-terminal amino acid of a polypeptide is designated as “*0aE+*0bV”. A “downstream” insertion can also be described as the addition of one or more amino acids between the named position and the position immediately following the named position, e.g. an insertion downstream of position 195 results in the addition of one or more amino acids between position 195 and 196, thereby generating new positions *195a, *195b and so forth.

### Parent Lysozymes

Any suitable parental lysozyme may be used in the present invention. Sources of parental lysozymes include, e.g., microbial (bacterial, fungal), viral, plant, insect, fish, and animal (vertebrate, such as avian, and mammalian). In an embodiment, the parental enzyme is avian. In a specific embodiment, the parental enzyme is derived from Opisthocomus hoazin.

In another embodiment, the parental lysozyme is (a) a polypeptide corresponding to the mature peptide of SEQ ID NO:2, SEQ ID NO:4 or SEQ ID NO:5 or to a polypeptide shown in SEQ ID NO:6; or (b) a polypeptide comprising an amino acid sequence having at least 80%, at least 85%, at least 90%, at least 95%, at least 96%, at least 97%, at least 98% at least 99% or 100% identity with the mature polypeptide of SEQ ID NO:2, SEQ ID NO:4 or SEQ ID NO:5 or to a polypeptide shown in SEQ ID NO:6; or (c) a polypeptide encoded by a polynucleotide that hybridizes under at least medium stringency conditions with (i) the mature polypeptide encoded by sequence of SEQ ID NO:1 or SEQ ID NO:3, (ii) the genomic or artificial DNA sequence comprising the mature polypeptide coding sequence of SEQ ID NO:1 or SEQ ID NO:3, or (iii) a full-length complementary strand of (i) or (ii); or (d) a polypeptide encoded by a polynucleotide comprising a nucleotide sequence having at least at least at least 80%, at least 85%, at least 90%, at least 95%, at least 96%, at least 97%, at least 98%, at least 99%, or 100% identity with the mature polypeptide coding sequence of SEQ ID NO:1 or SEQ ID NO:3.

1. The variant of claim 1, wherein the variant lysozyme comprises an amino acid sequence which is at least at least 85%, identical to the a polypeptide corresponding to the mature polypeptide of SEQ ID NO:2, SEQ ID NO:4 or SEQ ID NO:5 or to a polypeptide shown in SEQ ID NO:6.
2. The variant of claim 1, wherein the variant lysozyme comprises an amino acid sequence which is at least at least 90%, identical to the a polypeptide corresponding to the mature polypeptide of SEQ ID NO:2, SEQ ID NO:4 or SEQ ID NO:5 or to a polypeptide shown in SEQ ID NO:6.
3. The variant of claim 1, wherein the variant lysozyme comprises an amino acid sequence which is at least at least 93% identical to the a polypeptide corresponding to the mature polypeptide of SEQ ID NO:2, SEQ ID NO:4 or SEQ ID NO:5 or to a polypeptide shown in SEQ ID NO:6.
4. The variant of claim 1, wherein the variant lysozyme comprises an amino acid sequence which is at least at least 95% identical to the a polypeptide corresponding to the mature polypeptide of SEQ ID NO:2, SEQ ID NO:4 or SEQ ID NO:5 or to a polypeptide shown in SEQ ID NO:6.
5. The variant of claim 1, wherein the variant lysozyme comprises an amino acid sequence which is at least at least 97% identical to the a polypeptide corresponding to the mature polypeptide of SEQ ID NO:2, SEQ ID NO:4 or SEQ ID NO:5 or to a polypeptide shown in SEQ ID NO:6.
6. The variant of claim 1, wherein the variant lysozyme comprises an amino acid sequence which is at least at least 99% identical to the a polypeptide corresponding to the mature polypeptide of SEQ ID NO:2, SEQ ID NO:4 or SEQ ID NO:5 or to a polypeptide shown in SEQ ID NO:6.

In another embodiment, the parental enzyme comprises or consists of the amino acid sequence shown in SEQ ID NO:2, SEQ ID NO:4 or SEQ ID NO:5 or to a polypeptide shown in SEQ ID NO:6. In another embodiment, the parental enzyme is (a) a polypeptide comprising or consisting of the amino acid sequence of SEQ ID NO:2, SEQ ID NO:4 or SEQ ID NO:5 or to a polypeptide shown in SEQ ID NO:6; or (b) a polypeptide comprising an amino acid sequence having at least 80%, at least 85%, at least 90%, at least 95%, at least 96%, at least 97%, at least 98% at least 99% or 100% identity with the mature polypeptide of SEQ ID NO:2, SEQ ID NO:4 or SEQ ID NO:5 or to a polypeptide shown in SEQ ID NO:6.

In one embodiment, the parental lysozyme is (a) a polypeptide comprising an amino acid sequence having at least 80% identity with the mature polypeptide of SEQ ID NO:2, SEQ ID NO:4 or SEQ ID NO:5 or to a polypeptide shown in SEQ ID NO:6; or (b) a polypeptide encoded by a polynucleotide comprising a nucleotide sequence having at least at least at least 80% identity with the mature polypeptide coding sequence of SEQ ID NO:1 or SEQ ID NO:3.

In one embodiment, the parental enzyme is a polypeptide comprising an amino acid sequence having at least 80% identity with the mature polypeptide of SEQ ID NO:2, SEQ ID NO:4 or SEQ ID NO:5 or to a polypeptide shown in SEQ ID NO:6.

In one embodiment, the parental lysozyme is (a) a polypeptide comprising an amino acid sequence having at least 85% identity with the mature polypeptide of SEQ ID NO:2, SEQ ID NO:4 or SEQ ID NO:5 or to a polypeptide shown in SEQ ID NO:6; or (b) a polypeptide encoded by a polynucleotide comprising a nucleotide sequence having at least at least at least 85% identity with the mature polypeptide coding sequence of SEQ ID NO:1 or SEQ ID NO:3.

In one embodiment, the parental enzyme is a polypeptide comprising an amino acid sequence having at least 85% identity with the mature polypeptide of SEQ ID NO:2, SEQ ID NO:4 or SEQ ID NO:5 or to a polypeptide shown in SEQ ID NO:6.

In one embodiment, the parental lysozyme is (a) a polypeptide comprising an amino acid sequence having at least 90% identity with the mature polypeptide of SEQ ID NO:2, SEQ ID NO:4 or SEQ ID NO:5 or to a polypeptide shown in SEQ ID NO:6; or (b) a polypeptide encoded by a polynucleotide comprising a nucleotide sequence having at least at least at least 90% identity with the mature polypeptide coding sequence of SEQ ID NO:1 or SEQ ID NO:3.

In one embodiment, the parental enzyme is a polypeptide comprising an amino acid sequence having at least 90% identity with the mature polypeptide of SEQ ID NO:2, SEQ ID NO:4 or SEQ ID NO:5 or to a polypeptide shown in SEQ ID NO:6.

In one embodiment, the parental lysozyme is (a) a polypeptide comprising an amino acid sequence having at least 95% identity with the mature polypeptide of SEQ ID NO:2, SEQ ID NO:4 or SEQ ID NO:5 or to a polypeptide shown in SEQ ID NO:6; or (b) a polypeptide encoded by a polynucleotide comprising a nucleotide sequence having at least at least at least 95% identity with the mature polypeptide coding sequence of SEQ ID NO:1 or SEQ ID NO:3.

In one embodiment, the parental enzyme is a polypeptide comprising an amino acid sequence having at least 95% identity with the mature polypeptide of SEQ ID NO:2, SEQ ID NO:4 or SEQ ID NO:5 or to a polypeptide shown in SEQ ID NO:6.

In one embodiment, the parental lysozyme is (a) a polypeptide comprising an amino acid sequence having at least 97% identity with the mature polypeptide of SEQ ID NO:2, SEQ ID NO:4 or SEQ ID NO:5 or to a polypeptide shown in SEQ ID NO:6; or (b) a polypeptide encoded by a polynucleotide comprising a nucleotide sequence having at least at least at least 97% identity with the mature polypeptide coding sequence of SEQ ID NO:1 or SEQ ID NO:3.

In one embodiment, the parental enzyme is a polypeptide comprising an amino acid sequence having at least 97% identity with the mature polypeptide of SEQ ID NO:2, SEQ ID NO:4 or SEQ ID NO:5 or to a polypeptide shown in SEQ ID NO:6.

The parent lysozymes may in another aspect be encoded by polynucleotides that hybridize under very low stringency conditions, preferably low stringency conditions, more preferably medium stringency conditions, more preferably medium-high stringency conditions, even more preferably high stringency conditions, and most preferably very high stringency conditions with (i) the mature polypeptide coding sequence of SEQ ID NO:1 or SEQ ID NO:3, (ii) the genomic DNA sequence comprising the mature polypeptide coding sequence of SEQ ID NO:1 or SEQ ID NO:3, (iii) a subsequence of (i) or (ii), or (iv) a full-length complementary strand of (i), (ii), or (iii) (J. Sambrook, E. F. Fritsch, and T. Maniatis (1989) Molecular Cloning, A Laboratory Manual (2^{nd} edition), Cold Spring Harbor, N.Y.). The subsequence may encode a polypeptide fragment having antimicrobial and/or lysozyme activity. In one aspect, the complementary strand is the full-length complementary strand of the mature polypeptide coding sequence of SEQ ID NO:1 or SEQ ID NO:3. A subsequence of the mature polypeptide coding sequence of SEQ ID NO:1 or SEQ ID NO:3, or a homolog thereof, is a nucleotide sequence where one or more (several) nucleotides have been deleted from the 5′- and/or 3′-end, where the polypeptide encoded by the subsequence possess antimicrobial and/or lysozyme activity.

The polynucleotide of SEQ ID NO:1 or SEQ ID NO:3 or a fragment thereof may be used to design nucleic acid probes to identify and clone DNA encoding parent lysozymes from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, at least 25, at least 35, or at least 70 nucleotides in length. It is, however, preferred that the nucleic acid probe is at least 100 nucleotides in length. For example, the nucleic acid probe may be at least 200 nucleotides, at least 300 nucleotides, at least 400 nucleotides, or at least 500 nucleotides in length. Even longer probes may be used, e.g. nucleic acid probes that are at least 600 nucleotides, at least 700 nucleotides, at least 800 nucleotides, at least 900 nucleotides in length, at least 1000 nucleotides in length, at least 1100 nucleotides in length, at least 1200 nucleotides in length, at least 1300 nucleotides in length, at least 1400 nucleotides in length, at least 1500 nucleotides in length or at least 1600 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA library prepared from other organisms may be screened for DNA that hybridizes with the probes described above and encodes a parent lysozyme. Genomic or other DNA from other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO:1 or SEQ ID NO:3, or a subsequence thereof, the carrier material is used in a Southern blot. For purposes of the present invention, hybridization indicates that the polynucleotide hybridizes to a labeled nucleotide probe corresponding to the polynucleotide shown in SEQ ID NO:1 or SEQ ID NO:3, its complementary strand, or a subsequence thereof, under low to very high stringency conditions. Molecules to which the probe hybridizes can be detected using, for example, X-ray film or any other detection means known in the art.

In one aspect, the nucleic acid probe is the mature polypeptide coding sequence of SEQ ID NO:1 or SEQ ID NO:3. In another aspect, the nucleic acid probe is a polynucleotide sequence that encodes the polypeptide of SEQ ID NO:2, SEQ ID NO:4 or SEQ ID NO:5 or to a polypeptide shown in SEQ ID NO:6, or a subsequence thereof. In another aspect, the nucleic acid probe is SEQ ID NO:1 or SEQ ID NO:3.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at 45° C. (very low stringency), more preferably at 50° C. (low stringency), more preferably at 55° C. (medium stringency), more preferably at 60° C. (medium-high stringency), even more preferably at 65° C. (high stringency), and most preferably at 70° C. (very high stringency).

For short probes that are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (see Bolton and McCarthy (1962) Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short probes that are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In another embodiment, the parent lysozyme is encoded by a polynucleotide comprising or consisting of a nucleotide sequence having a degree of identity to the mature polypeptide coding sequence of SEQ ID NO:1 or SEQ ID NO:3 of preferably at least 65%, more preferably at least 70%, more preferably at least 75%, more preferably at least 80%, more preferably at least 85%, even more preferably at least 90%, most preferably at least 95%, and even most preferably 96%, 97%, 98%, or 99%, which encode an active polypeptide.

### Generation of Variants

Variants of a parent lysozyme can be prepared according to any mutagenesis procedure known in the art, such as random and/or site-directed mutagenesis, synthetic gene construction, semi-synthetic gene construction, random mutagenesis, shuffling, etc.

Synthetic gene construction entails in vitro synthesis of a designed polynucleotide molecule to encode a polypeptide molecule of interest. Gene synthesis can be performed utilizing a number of techniques, such as the multiplex microchip-based technology described by Tian et al. (Tian et al., Nature 432:1050-1054) and similar technologies wherein oligonucleotides are synthesized and assembled upon photo-programmable microfluidic chips.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide fragments may then be shuffled.

Site-directed mutagenesis is a technique in which one or several mutations are created at a defined site in a polynucleotide molecule encoding the parent lysozyme. The technique can be performed in vitro or in vivo.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent lysozyme and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. For further description of suitable techniques reference is made to: Sambrook et al. (1989) Molecular cloning: A laboratory manual, Cold Spring Harbor lab., Cold Spring Harbor, N.Y.; Ausubel, F. M. et al. (eds.) Current protocols in Molecular Biology, John Wiley and Sons (1995); Harwood, C. R., and Cutting, S. M. (eds.) Molecular Biological Methods for Bacillus, John Wiley and Sons (1990); WO 96/34946; Scherer and Davis (1979) Proc. Natl. Acad. Sci. USA 76:4949-4955; and Barton et al. (1990) Nucleic Acids Research 18:7349-4966.

After the ligase reaction the ligation mixture may be used to transform a host cell, for cloning purposes E. coli cells are often used as described in Ausubel, F. M. et al. The transformed E. coli cells can be propagated in liquid media or on solid agar plates, plasmids can be rescued from the transformed cells and used to transform B. subtilis cells. Suitable competent Bacillus cells, such as MB1510, a 168-derivative (e.g. available from BGSC with accession no. 1A1 168 trpC2), may be transformed as described in WO 03/095658. An E. coli plasmid-borne integration cassette for library construction may be used for Bacillus transformation. The method is described in detail in WO 03/095658. Alternatively, an in vitro amplified PCR-SOE-product (Melnikov and Youngman, Nucleic Acid Research 27:1056) may be used.

Site-directed mutagenesis can be accomplished in vivo by methods known in the art (see for example U.S. Patent Application Publication 2004/0171154; Storici et al. (2001) Nature Biotechnology 19: 773-776; Kren et al. (1998) Nat. Med. 4: 285-290; and Calissano and Macino (1996) Fungal Genet. Newslett. 43: 15-16).

Any site-directed mutagenesis procedure can be used in the present invention. There are many commercial kits available that can be used to prepare variants of parent lysozymes.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer (1988) Science 241:53-57; Bowie and Sauer (1989) Proc. Natl. Acad. Sci. USA 86:2152-2156; WO 95/17413; or WO 95/22625. Other methods that can be used include error-prone PCR, phage display (e.g. Lowman et al. (1991) Biochem. 30:10832-10837; U.S. Pat. No. 5,223,409; WO 92/06204) and region-directed mutagenesis (Derbyshire et al. (1986) Gene 46:145; Ner et al. (1988) DNA 7:127).

Mutagenesis/shuffling methods as described above can be combined with high-throughput, automated screening methods to detect the activity of cloned, mutagenized polypeptides expressed by host cells, e.g. Bacillus as described above. Mutagenized DNA molecules that encode polypeptides with antimicrobial and/or lysozyme activity can be recovered from the host cells and rapidly sequenced using standard methods in the art.

### Variants

The lysozyme variants of the present invention comprise or consist of an alteration of an amino acid sequence at one or more positions selected from the group consisting of position number 3, 9, 10, 11, 17, 20, 25, 29, 32, 34, 37, 41, 47, 50, 60, 61, 67, 69, 73, 74, 81, 87, 88, 89, 90, 91, 92, 96, 97, 98, 101, 104, 108, 109, 111, and/or 124, which position corresponds to a position in amino acid sequence SEQ ID NO:5. The lysozyme variants have antimicrobial and/or lysozyme activity.

The lysozyme variants of the present invention comprises or consists of a polypeptide comprising an amino acid sequence having at least 80%, at least 85%, at least 90%, at least 95%, at least 96%, at least 97%, at least 98% or at least 99% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 80% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 85% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 90% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 95% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 96% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 97% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 98% identity with the mature polypeptide of SEQ ID NO:2, SEQ ID NO:4 or SEQ ID NO:5 or to a polypeptide shown in SEQ ID NO:6.

The lysozyme variants of the present invention comprises or consists of a polypeptide comprising an amino acid sequence having at least 99% identity with the mature polypeptide of SEQ ID NO:2, SEQ ID NO:4 or SEQ ID NO:5 or to a polypeptide shown in SEQ ID NO:6.

In one embodiment, the isolated variant of the present invention comprises or consists of an alteration at position 3 (using SEQ ID NO:5 for numbering). In an embodiment, the alteration is a substitution of an amino acid at position 3 (using SEQ ID NO:5 for numbering) which improves the stability of the lysozyme. In an embodiment, the alteration is a substitution of the amino acid at position 3 with Phe. For example, the isolated variants of the present invention comprise or consist of an alteration of 3F or specifically 13F.

In one embodiment, the isolated variant of the present invention comprises or consists of an alteration at position 9 (using SEQ ID NO:5 for numbering). In an embodiment, the alteration is a substitution of an amino acid at position 9 (using SEQ ID NO:5 for numbering) which improves the stability of the lysozyme. In an embodiment, the alteration is a substitution of the amino acid at position 9 with Ala. For example, the isolated variants of the present invention comprise or consist of an alteration of 9A or specifically V9A.

In one embodiment, the isolated variant of the present invention comprises or consists of an alteration at position 10 (using SEQ ID NO:5 for numbering). In an embodiment, the alteration is a substitution of an amino acid at position 10 (using SEQ ID NO:5 for numbering) which reduces the glycation susceptibility of the lysozyme. In an embodiment, the alteration is a substitution of the amino acid at position 10 with Arg. For example, the isolated variants of the present invention comprise or consist of an alteration of 10R or specifically K10R.

In one embodiment, the isolated variant of the present invention comprises or consists of an alteration at position 11 (using SEQ ID NO:5 for numbering). In an embodiment, the alteration is a substitution of an amino acid at position 11 (using SEQ ID NO:5 for numbering) which improves the stability of the lysozyme. In an embodiment, the alteration is a substitution of the amino acid at position 11 with Thr. For example, the isolated variants of the present invention comprise or consist of an alteration of 11T or specifically 111T.

In one embodiment, the isolated variant of the present invention comprises or consists of an alteration at position 17 (using SEQ ID NO:5 for numbering). In an embodiment, the alteration is a substitution of an amino acid at position 17 (using SEQ ID NO:5 for numbering) which improves the stability of the lysozyme. In an embodiment, the alteration is a substitution of the amino acid at position 17 with Leu. For example, the isolated variants of the present invention comprise or consist of an alteration of 17L or specifically F17L.

In one embodiment, the isolated variant of the present invention comprises or consists of an alteration at position 20 (using SEQ ID NO:5 for numbering). In an embodiment, the alteration is a substitution of an amino acid which improves the stability of the enzyme. In an embodiment, the alteration is a substitution of the amino acid at position 20 with Tyr. For example, the isolated variants of the present invention comprise or consist of an alteration of 20Y or specifically F20Y.

In one embodiment, the isolated variant of the present invention comprises or consists of an alteration at position 25 (using SEQ ID NO:5 for numbering). In an embodiment, the alteration is a substitution of an amino acid at position 25 (using SEQ ID NO:5 for numbering) which improves the stability of the lysozyme. In an embodiment, the alteration is a substitution of the amino acid at position 25 with Leu. For example, the isolated variants of the present invention comprise or consist of an alteration of 25L or specifically I25L.

In one embodiment, the isolated variant of the present invention comprises or consists of an alteration at position 29 (using SEQ ID NO:5 for numbering). In an embodiment, the alteration is a substitution of an amino acid at position 29 (using SEQ ID NO:5 for numbering) which improves the stability of the lysozyme. In an embodiment, the alteration is a substitution of the amino acid at position 29 with Leu. For example, the isolated variants of the present invention comprise or consist of an alteration of 29L or specifically I29L.

In one embodiment, the isolated variant of the present invention comprises or consists of an alteration at position 32 (using SEQ ID NO:5 for numbering). In an embodiment, the alteration is a substitution of an amino acid at position 32 (using SEQ ID NO:5 for numbering) which improves the stability of the enzyme. In an embodiment, the alteration is a substitution of the amino acid at position 32 with Ala. For example, the isolated variants of the present invention comprise or consist of an alteration of 32A or specifically V32A.

In one embodiment, the isolated variant of the present invention comprises or consists of an alteration at position 34 (using SEQ ID NO:5 for numbering). In an embodiment, the alteration is a substitution of an amino acid at position 34 (using SEQ ID NO:5 for numbering) which improves the stability of the enzyme. In an embodiment, the alteration is a substitution of the amino acid at position 34 with Trp. For example, the isolated variants of the present invention comprise or consist of an alteration of 34W or specifically H34W.

In one embodiment, the isolated variant of the present invention comprises or consists of an alteration at position 37 (using SEQ ID NO:5 for numbering). In an embodiment, the alteration is a substitution of an amino acid at position 37 (using SEQ ID NO:5 for numbering) which improves the stability of the enzyme at alkaline pH. In an embodiment, the alteration is a substitution of the amino acid at position 37 with Asn. For example, the isolated variants of the present invention comprise or consist of an alteration of 37N or specifically D37N.

In one embodiment, the isolated variant of the present invention comprises or consists of an alteration at position 41 (using SEQ ID NO:5 for numbering). In an embodiment, the alteration is a substitution of an amino acid at position 41 (using SEQ ID NO:5 for numbering) which improves the stability of the enzyme at alkaline pH. In an embodiment, the alteration is a substitution of the amino acid at position 41 with Asn or Arg. For example, the isolated variants of the present invention comprise or consist of an alteration of 41N or specifically E41N, or 41R or specifically E41R.

In one embodiment, the isolated variant of the present invention comprises or consists of an alteration at position 47 (using SEQ ID NO:5 for numbering). In an embodiment, the alteration is a substitution of an amino acid at position 47 (using SEQ ID NO:5 for numbering) which improves the stability of the enzyme. In an embodiment, the alteration is a substitution of the amino acid at position 47 with Pro. For example, the isolated variants of the present invention comprise or consist of an alteration of 47P or specifically G47P.

In one embodiment, the isolated variant of the present invention comprises or consists of an alteration at position 50 (using SEQ ID NO:5 for numbering). In an embodiment, the alteration is a substitution of an amino acid at position 50 (using SEQ ID NO:5 for numbering) which alters the pH-dependent activity profile. In an embodiment, the alteration is a substitution of the amino acid at position 50 with Thr. For example, the isolated variants of the present invention comprise or consist of an alteration of 50T or specifically R50T.

In one embodiment, the isolated variant of the present invention comprises or consists of an alteration at position 60 (using SEQ ID NO:5 for numbering). In an embodiment, the alteration is a substitution of an amino acid at position 60 (using SEQ ID NO:5 for numbering) which reduces the glycation susceptibility of the lysozyme. In an embodiment, the alteration is a substitution of the amino acid at position 60 with Arg. For example, the isolated variants of the present invention comprise or consist of an alteration of 60R or specifically K60R.

In one embodiment, the isolated variant of the present invention comprises or consists of an alteration at position 61 (using SEQ ID NO:5 for numbering). In an embodiment, the alteration is a substitution of an amino acid at position 61 (using SEQ ID NO:5 for numbering) which improves the substrate specificity of the enzyme. In an embodiment, the alteration is a substitution of the amino acid at position 61 with Trp. For example, the isolated variants of the present invention comprise or consist of an alteration of 61W or specifically Y61W.

In one embodiment, the isolated variant of the present invention comprises or consists of an alteration at position 67 (using SEQ ID NO:5 for numbering). In an embodiment, the alteration is a substitution of an amino acid at position 67 (using SEQ ID NO:5 for numbering) which reduces the glycation susceptibility of the lysozyme. In an embodiment, the alteration is a substitution of the amino acid at position 67 with Arg. For example, the isolated variants of the present invention comprise or consist of an alteration of 67R or specifically K67R.

In one embodiment, the isolated variant of the present invention comprises or consists of an alteration at position 69 (using SEQ ID NO:5 for numbering). In an embodiment, the alteration is a substitution of an amino acid at position 69 (using SEQ ID NO:5 for numbering) which improves the stability of the enzyme at alkaline pH. In an embodiment, the alteration is a substitution of the amino acid at position 69 with Pro. For example, the isolated variants of the present invention comprise or consist of an alteration of 69P or specifically S69P.

In one embodiment, the isolated variant of the present invention comprises or consists of an alteration at position 73 (using SEQ ID NO:5 for numbering). In an embodiment, the alteration is a substitution of an amino acid at position 73 (using SEQ ID NO:5 for numbering) which improves the stability of the enzyme at alkaline pH. In an embodiment, the alteration is a substitution of the amino acid at position 73 with Asn. For example, the isolated variants of the present invention comprise or consist of an alteration of 73N or specifically D73N.

In one embodiment, the isolated variant of the present invention comprises or consists of an alteration at position 74 (using SEQ ID NO:5 for numbering). In an embodiment, the alteration is a substitution of an amino acid which improves the stability of the enzyme. In an embodiment, the alteration is a substitution of the amino acid at position 74 with Ala. For example, the isolated variants of the present invention comprise or consist of an alteration of 74A or specifically G74A.

In one embodiment, the isolated variant of the present invention comprises or consists of an alteration at position 81 (using SEQ ID NO:5 for numbering). In an embodiment, the alteration is a substitution of an amino acid at position 81 (using SEQ ID NO:5 for numbering) which improves the stability of the enzyme at alkaline pH. In an embodiment, the alteration is a substitution of the amino acid at position 81 with Ala. For example, the isolated variants of the present invention comprise or consist of an alteration of 81A or specifically E81A.

In one embodiment, the isolated variant of the present invention comprises or consists of an alteration at position 87 (using SEQ ID NO:5 for numbering). In an embodiment, the alteration is a substitution of an amino acid at position 87 (using SEQ ID NO:5 for numbering) which improves the stability of the lysozyme. In an embodiment, the alteration is a substitution of the amino acid at position 87 with Ile. For example, the isolated variants of the present invention comprise or consist of an alteration of 87I or specifically L87I.

In one embodiment, the isolated variant of the present invention comprises or consists of an alteration at position 88 (using SEQ ID NO:5 for numbering). In an embodiment, the alteration is a substitution of an amino acid at position 88 (using SEQ ID NO:5 for numbering) which improves the stability of the enzyme at alkaline pH. In an embodiment, the alteration is a substitution of the amino acid at position 88 with Thr. For example, the isolated variants of the present invention comprise or consist of an alteration of 88T or specifically E88T.

In one embodiment, the isolated variant of the present invention comprises or consists of an alteration at position 89 (using SEQ ID NO:5 for numbering). In an embodiment, the alteration is a substitution of an amino acid at position 89 (using SEQ ID NO:5 for numbering) which improves the stability of the enzyme at alkaline pH. In an embodiment, the alteration is a substitution of the amino acid at position 89 with Gln. For example, the isolated variants of the present invention comprise or consist of an alteration of 89Q or specifically D89Q.

In one embodiment, the isolated variant of the present invention comprises or consists of an alteration at position 90 (using SEQ ID NO:5 for numbering). In an embodiment, the alteration is a substitution of an amino acid at position 90 (using SEQ ID NO:5 for numbering) which alters the pH-dependent activity profile. In an embodiment, the alteration is a substitution of the amino acid at position 90 with Ala. For example, the isolated variants of the present invention comprise or consist of an alteration of 90A or specifically D90A.

In one embodiment, the isolated variant of the present invention comprises or consists of an alteration at position 91 (using SEQ ID NO:5 for numbering). In an embodiment, the alteration is a substitution of an amino acid at position 91 (using SEQ ID NO:5 for numbering) which improves the stability of the lysozyme. In an embodiment, the alteration is a substitution of the amino acid at position 91 with Val. For example, the isolated variants of the present invention comprise or consist of an alteration of 91V or specifically I91V.

In one embodiment, the isolated variant of the present invention comprises or consists of an alteration at position 92 (using SEQ ID NO:5 for numbering). In an embodiment, the alteration is a substitution of an amino acid at position 92 (using SEQ ID NO:5 for numbering) which reduces the glycation susceptibility of the lysozyme. In an embodiment, the alteration is a substitution of the amino acid at position 92 with Ala. For example, the isolated variants of the present invention comprise or consist of an alteration of 92A or specifically K92A.

In one embodiment, the isolated variant of the present invention comprises or consists of an alteration at position 96 (using SEQ ID NO:5 for numbering). In an embodiment, the alteration is a substitution of an amino acid at position 96 (using SEQ ID NO:5 for numbering) which reduces the glycation susceptibility of the lysozyme. In an embodiment, the alteration is a substitution of the amino acid at position 96 with Arg. For example, the isolated variants of the present invention comprise or consist of an alteration of 96R or specifically K96R.

In one embodiment, the isolated variant of the present invention comprises or consists of an alteration at position 97 (using SEQ ID NO:5 for numbering). In an embodiment, the alteration is a substitution of an amino acid at position 97 (using SEQ ID NO:5 for numbering) which improves the substrate specificity of the enzyme. In an embodiment, the alteration is a substitution of the amino acid at position 97 with Val. For example, the isolated variants of the present invention comprise or consist of an alteration of 97V or specifically I97V.

In one embodiment, the isolated variant of the present invention comprises or consists of an alteration at position 98 (using SEQ ID NO:5 for numbering). In an embodiment, the alteration is a substitution of an amino acid at position 98 (using SEQ ID NO:5 for numbering) which improves the stability of the lysozyme. In an embodiment, the alteration is a substitution of the amino acid at position 98 with Val. For example, the isolated variants of the present invention comprise or consist of an alteration of 98V or specifically A98V.

In one embodiment, the isolated variant of the present invention comprises or consists of an alteration at position 101 (using SEQ ID NO:5 for numbering). In an embodiment, the alteration is a substitution of an amino acid at position 101 (using SEQ ID NO:5 for numbering) which improves the stability of the enzyme at alkaline pH. In an embodiment, the alteration is a substitution of the amino acid at position 101 with Pro. For example, the isolated variants of the present invention comprise or consist of an alteration of 101P or specifically A101P.

In one embodiment, the isolated variant of the present invention comprises or consists of an alteration at position 104 (using SEQ ID NO:5 for numbering). In an embodiment, the alteration is a substitution of an amino acid at position 104 (using SEQ ID NO:5 for numbering) which improves the stability of the lysozyme. In an embodiment, the alteration is a substitution of the amino acid at position 104 with Ile. For example, the isolated variants of the present invention comprise or consist of an alteration of 104I or specifically L104I.

In one embodiment, the isolated variant of the present invention comprises or consists of an alteration at position 108 (using SEQ ID NO:5 for numbering). In an embodiment, the alteration is a substitution of an amino acid at position 108 (using SEQ ID NO:5 for numbering) which improves the substrate specificity of the enzyme. In an embodiment, the alteration is a substitution of the amino acid at position 108 with Val. For example, the isolated variants of the present invention comprise or consist of an alteration of 108V or specifically Y108V.

In one embodiment, the isolated variant of the present invention comprises or consists of an alteration at position 109 (using SEQ ID NO:5 for numbering). In an embodiment, the alteration is a substitution of an amino acid at position 109 (using SEQ ID NO:5 for numbering) which improves the substrate specificity of the enzyme. In an embodiment, the alteration is a substitution of an amino acid which improves the stability of the enzyme. In an embodiment, the alteration is a substitution of the amino acid at position 109 with Val. For example, the isolated variants of the present invention comprise or consist of an alteration of 109A or specifically G109A.

In one embodiment, the isolated variant of the present invention comprises or consists of an alteration at position 111 (using SEQ ID NO:5 for numbering). In an embodiment, the alteration is a substitution of an amino acid at position 111 (using SEQ ID NO:5 for numbering) which reduces the glycation susceptibility of the lysozyme. In an embodiment, the alteration is a substitution of the amino acid at position 111 with Arg. For example, the isolated variants of the present invention comprise or consist of an alteration of 111R or specifically K111R.

In one embodiment, the isolated variant of the present invention comprises or consists of an alteration at position 124 (using SEQ ID NO:5 for numbering). In an embodiment, the alteration is a substitution of an amino acid at position 124 (using SEQ ID NO:5 for numbering) which reduces the glycation susceptibility of the lysozyme. In an embodiment the alteration is a substitution of the amino acid at position 124 with Arg. For example, the isolated variants of the present invention comprise or consist of an alteration of K124R. In an embodiment, the alteration is a substitution of an amino acid which improves the stability of the enzyme. In an embodiment, the alteration is a substitution of the amino acid at position 124 with Ser or Thr. For example, the isolated variants of the present invention comprise or consist of an alteration of 124S or specifically K124S, or 124T or specifically K124T.

### Functional Characteristics

The lysozyme variants of the present invention are further defined as having antimicrobial and/or lysozyme activity. Lysozyme variants which are not capable of catalyzing the hydrolysis of 1,4-beta-linkages between N-acetylmuramic acid and N-acetyl-D-glucosamine residues in a peptidoglycan and between N-acetyl-D-glucosamine residues in chitodextrins may still have an antimicrobial effect since such inactive lysozyme variants can bind to the surface of the microorganism and potentially inhibit its growth. Such lysozyme variants may also be termed “bacteriostatics”.

### Structural Characteristics

The lysozyme variants are further defined as (a) a polypeptide having an amino acid sequence which is at least 80%, at least 85%, at least 90%, at least 95%, at least 96%, at least 97%, at least 98%, or at least 99% identity with the mature polypeptide of SEQ ID NO:3 or with the polypeptide of SEQ ID NO:5 or SEQ ID NO:6; or (b) a polypeptide encoded by a polynucleotide that hybridizes under at least medium stringency conditions with (i) the mature polypeptide encoded by sequence of SEQ ID NO:10R SEQ ID NO:3 (ii) the genomic or artificial DNA sequence comprising the mature polypeptide coding sequence of SEQ ID NO:10R SEQ ID NO:3, or (iii) a full-length complementary strand of (i) or (ii); or (d) a polypeptide encoded by a polynucleotide comprising a nucleotide sequence having at least 60%, at least 65%, at least 70%, at least 75%, at least 80%, at least 85%, at least 90%, at least 95%, at least 96%, at least 97%, at least 98%, or at least 99% identity with the mature polypeptide coding sequence of SEQ ID NO:10R SEQ ID NO:3.

In one aspect, the variants of the present invention have an amino acid sequence that differs by thirty amino acids, twenty nine amino acids, twenty eight amino acids, twenty seven amino acids, twenty six amino acids, twenty five amino acids, twenty four amino acids, twenty three amino acids, twenty two amino acids, twenty amino acids, nineteen amino acids, eighteen amino acids, seventeen amino acids, sixteen amino acids, fifteen amino acids, fourteen amino acids, thirteen amino acids, twelve amino acids, eleven amino acids, ten amino acids, nine amino acids, eight amino acids, seven amino acids, six amino acids, five amino acids, four amino acids, three amino acids, two amino acids, or only one amino acid from the parent lysozyme or reference sequence (e.g., the mature polypeptide of SEQ ID NO:2 or the polypeptide of SEQ ID NO:5).

In addition to the alterations described herein, the lysozyme variants may also comprise other alterations, such as, e.g., a conservative amino acid substitution and other substitutions that do not significantly affect the three-dimensional folding or activity of the protein or polypeptide. Amino acid substitutions which do not generally alter specific activity are known in the art and are described, for example, by Neurath and Hill (1979) The Proteins, Academic Press, New York. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

The lysozyme variants may also comprise small deletions, typically of one to about 9 amino acids, one to 15 amino acids or one to 30 amino acids; and small amino- or carboxyl-terminal extensions, such as an amino-terminal methionine residue, a small linker peptide of up to about five to ten residues, 10 to 15 residues, or 20 to 25 residues, or a small extension that facilitates purification (an affinity tag), such as a poly-histidine tag, or protein A (Nilsson et al. (1985) EMBO J. 4:1075; Nilsson et al. (1991) Methods Enzymol. 198:3. See also, in general, Ford et al. (1991) Protein Expression and Purification 2:95-107.

Although the minor changes described may be applied to the lysozyme variants, such changes may also be of a substantive nature, such as, other alterations known in the art to change or improve one or more properties of the lysozyme variants, in particular, alterations known in the GH22 hydrolase family lysozymes. The lysozyme variants may also include fusion of larger polypeptides (e.g., up to 300 amino acids or more) both as amino- or carboxyl-terminal extensions. Examples of fusion polypeptides include the addition of a binding domains and/or linker segments to the lysozymes of the present invention.

Essential amino acids in the lysozyme polypeptides of the present invention can be identified according to procedures known in the art, such as site-directed mutagenesis or alanine-scanning mutagenesis (Cunningham and Wells (1989) Science 244:1081-1085). In the latter technique, single alanine mutations are introduced at every residue in the molecule, and the resultant mutant molecules are tested for biological activity (i.e. antimicrobial and/or lysozyme activity) to identify amino acid residues that are critical to the activity of the molecule. See also Hilton et al. (1996) J. Biol. Chem. 271:4699-4708. The active site of the enzyme or other biological interaction can also be determined by physical analysis of structure, as determined by such techniques as nuclear magnetic resonance, crystallography, electron diffraction or photo affinity labeling, in conjunction with mutation of putative contact site amino acids. See for example de Vos et al. (1992) Science 255:306-312; Smith et al. (1992) J. Mol. Biol. 224:899-904; Wlodaver et al. (1992) FEBS Lett. 309:59-64. The identities of essential amino acids can also be inferred from analysis of homologies with polypeptides which are related to a polypeptide according to the invention.

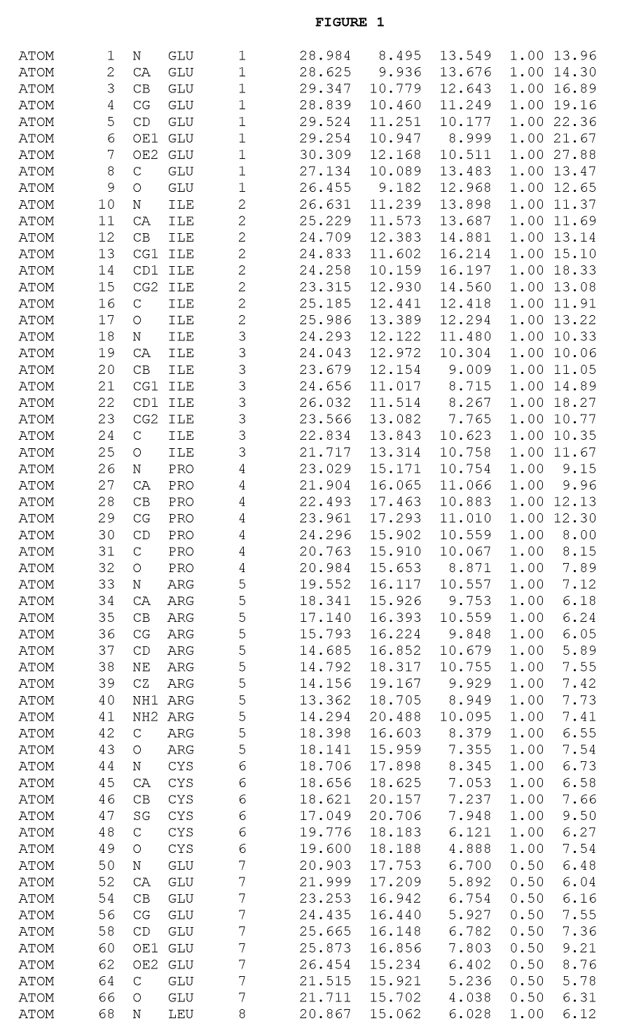

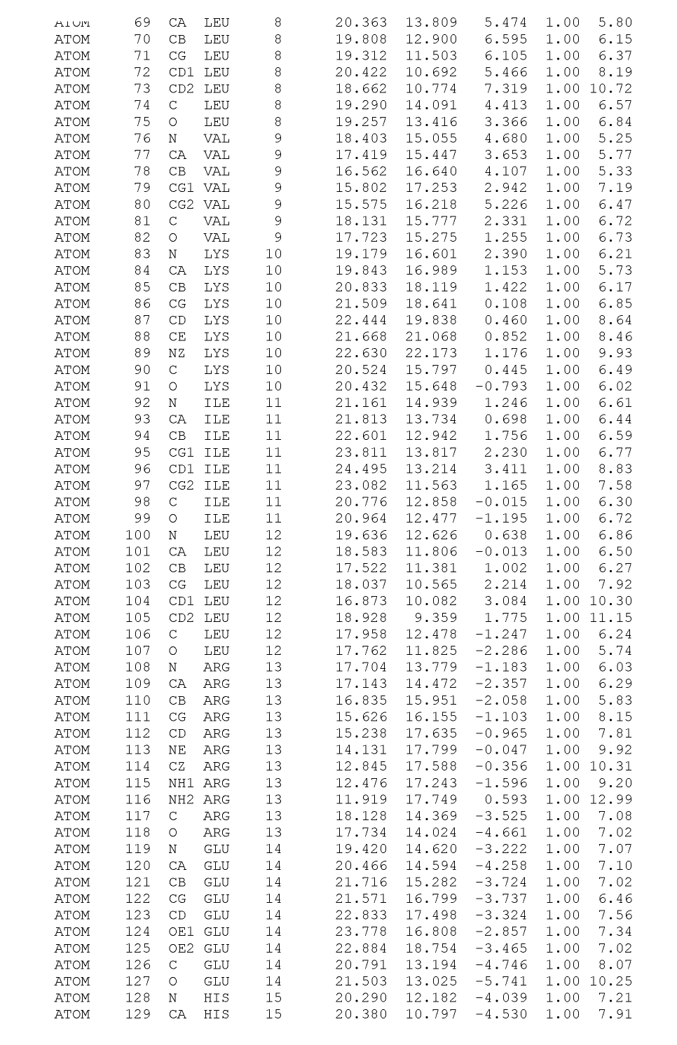

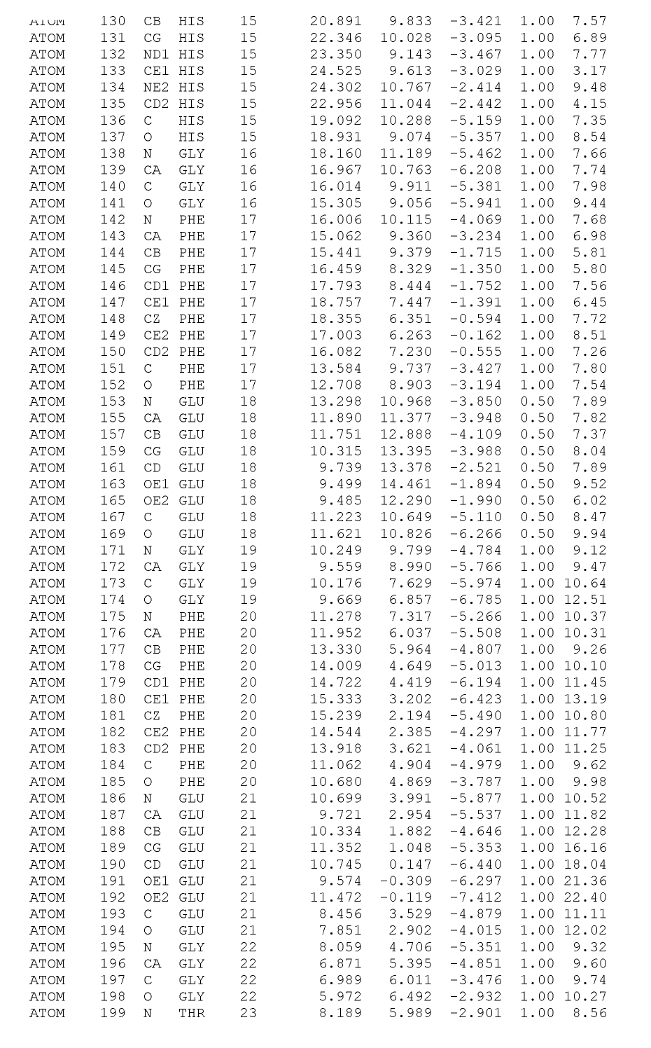

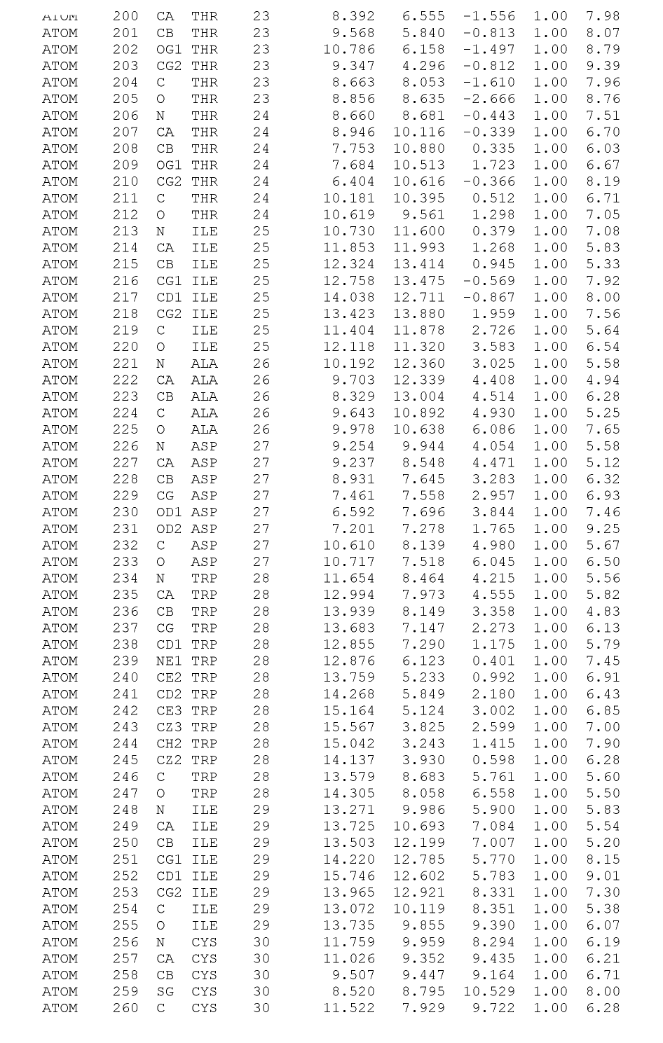

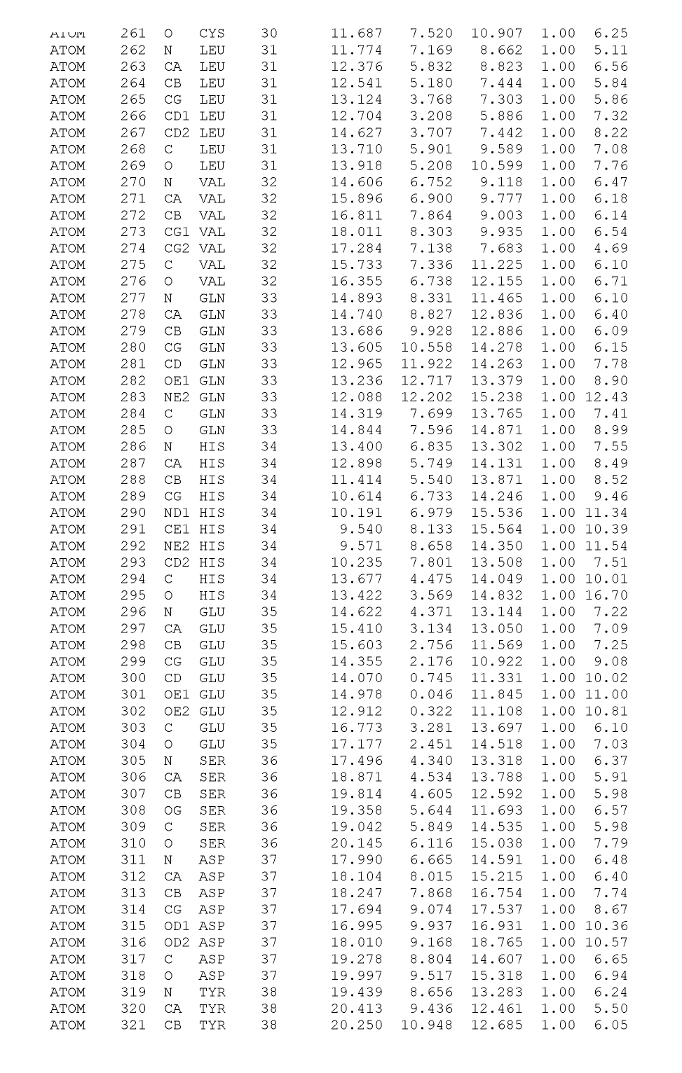

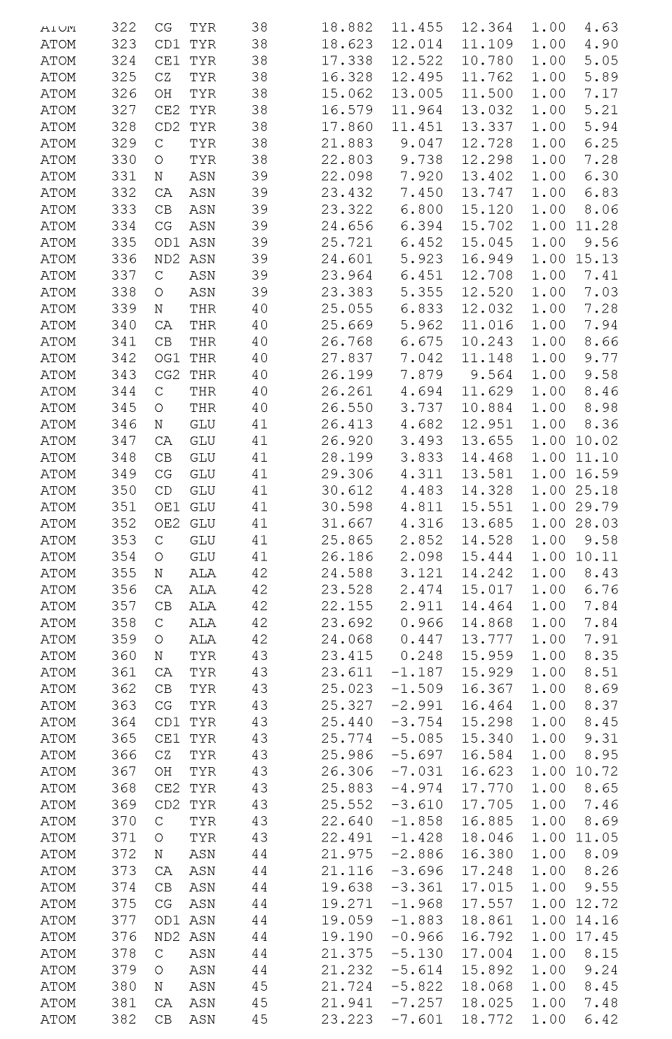

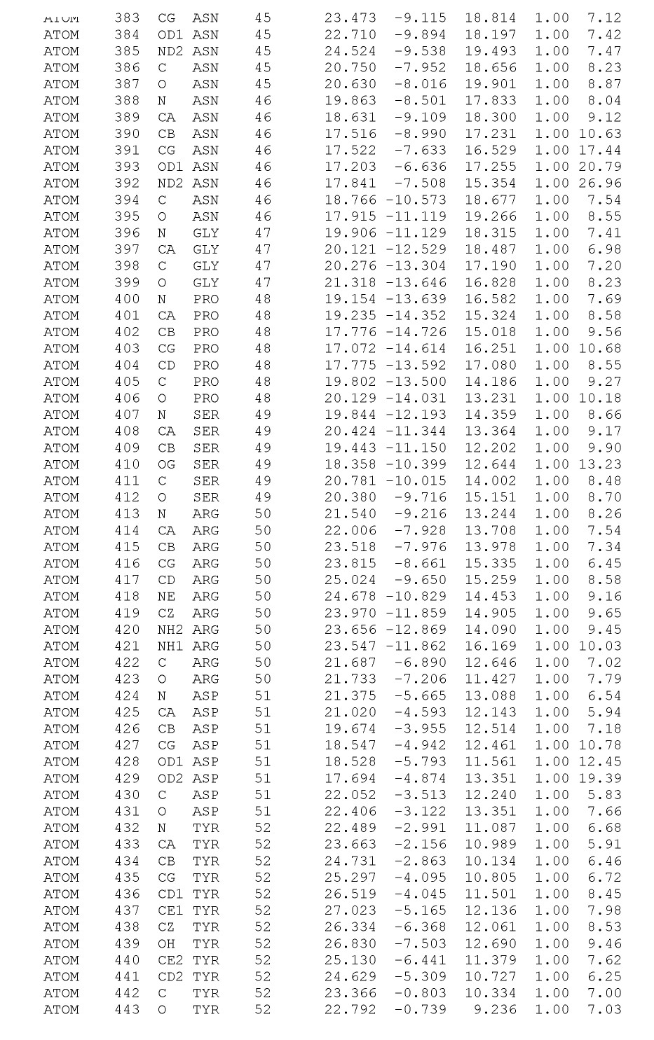

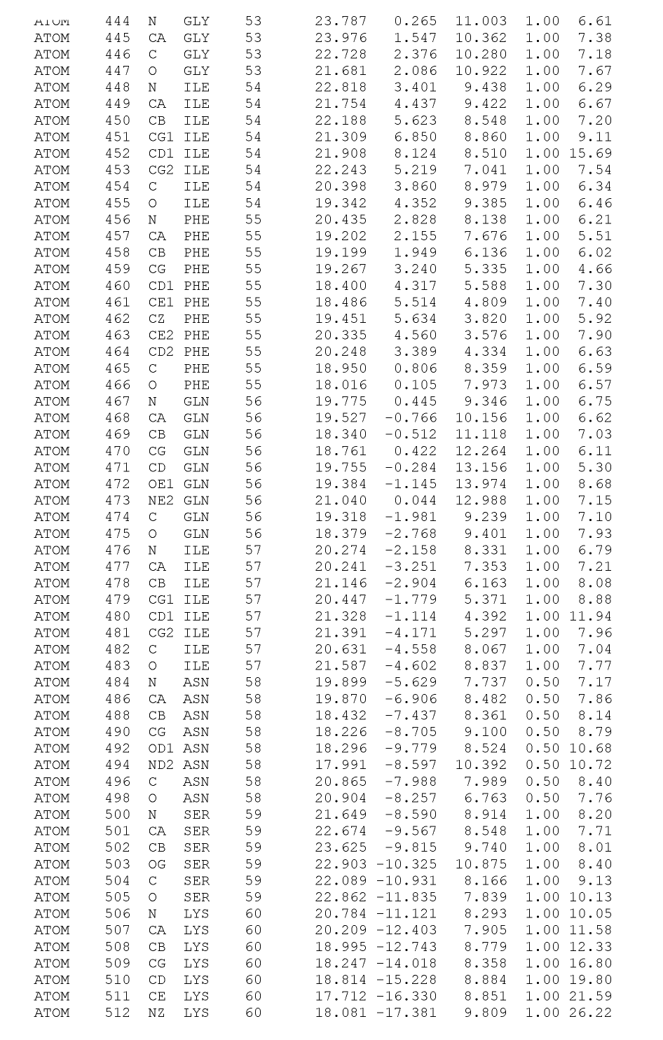

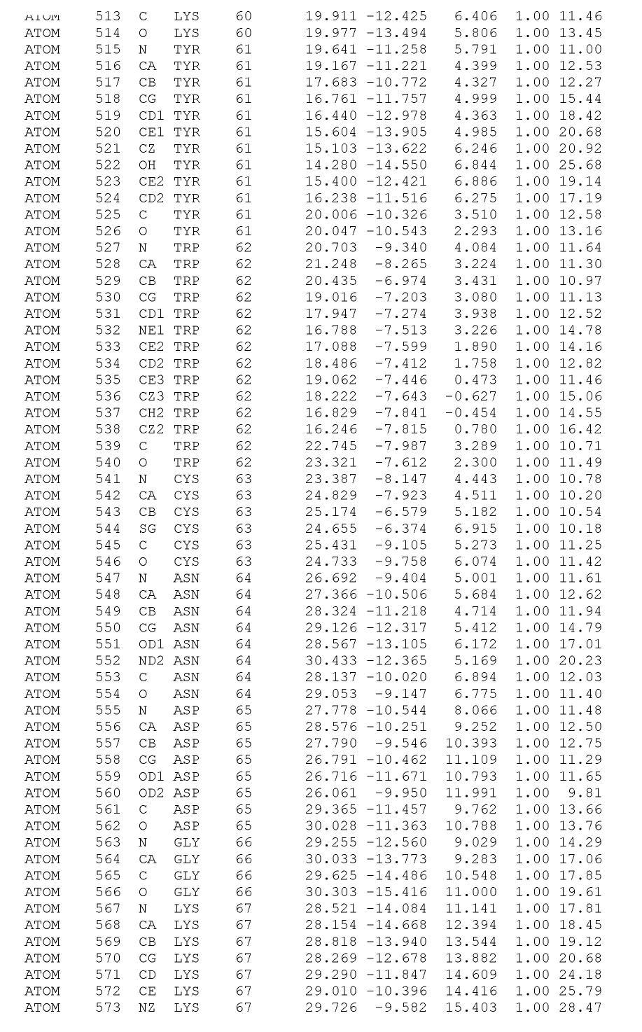

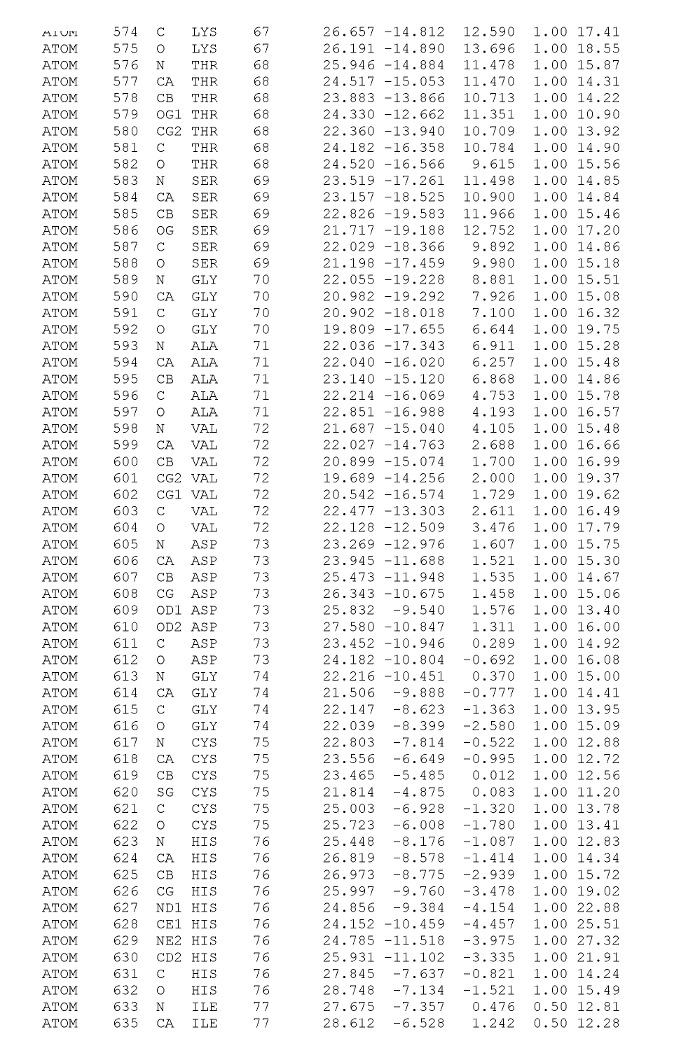

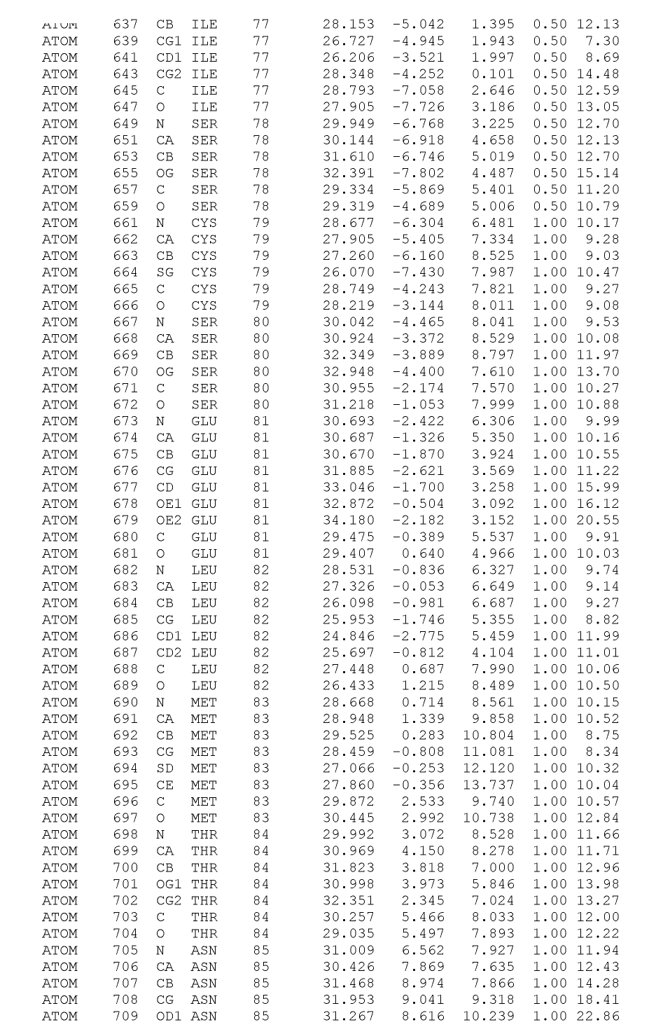

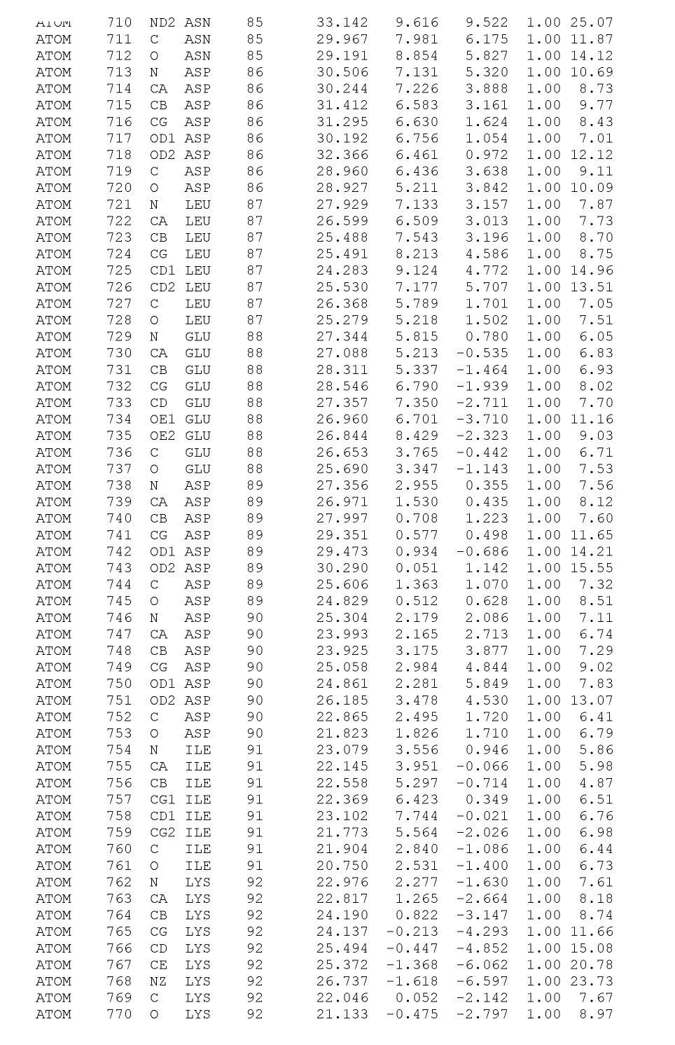

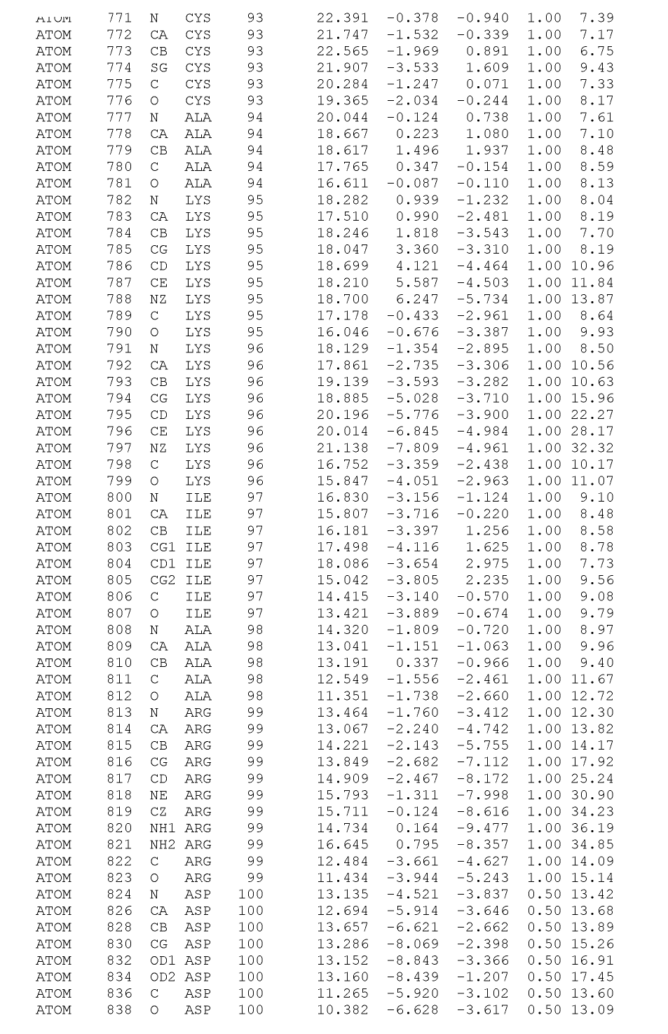

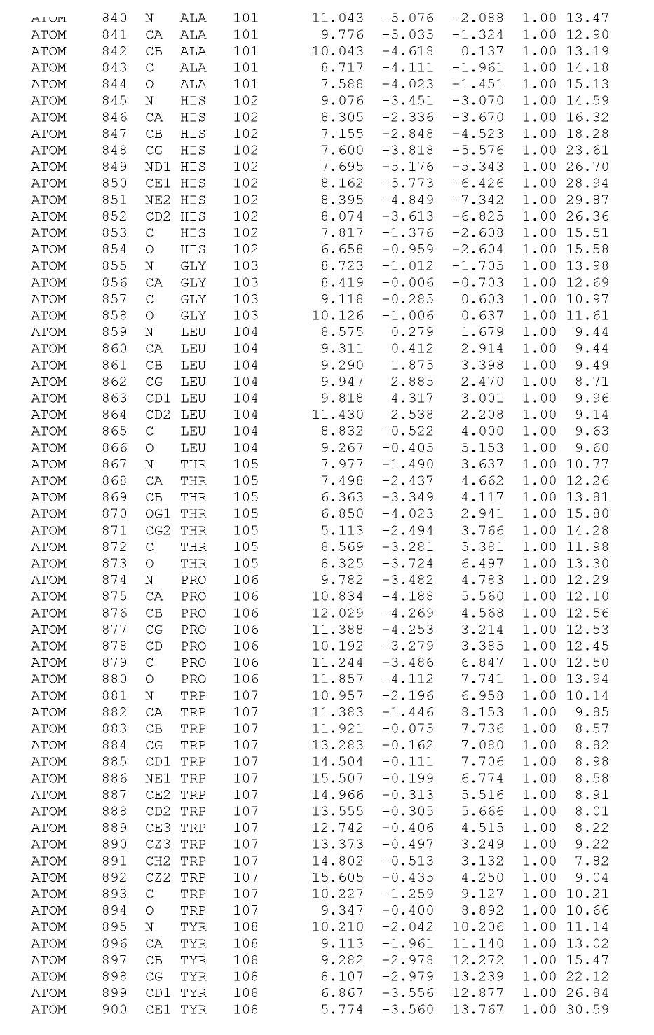

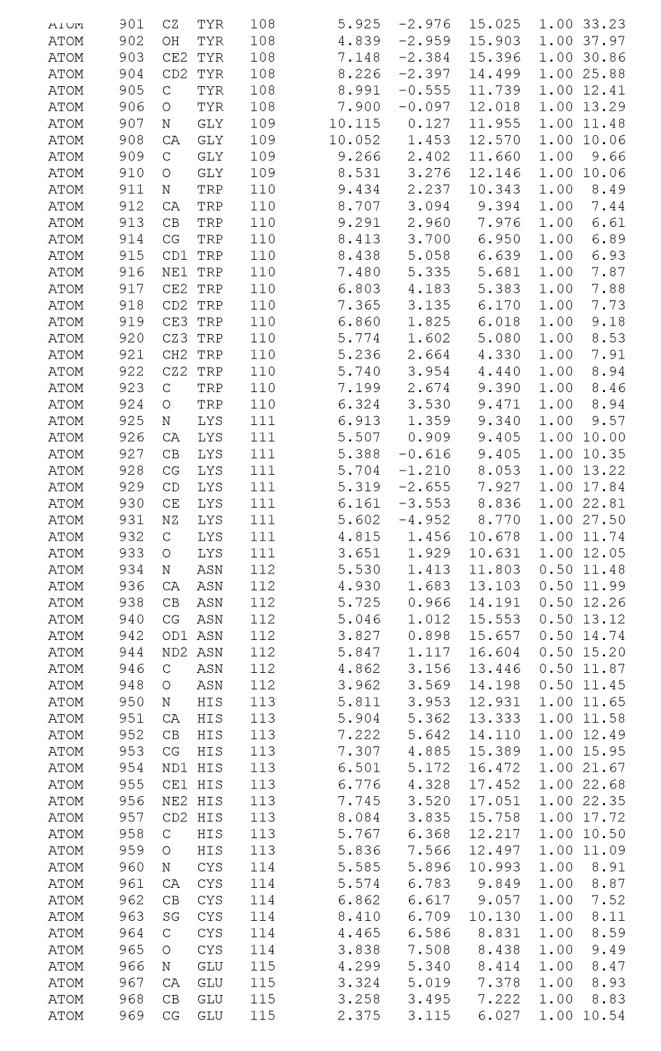

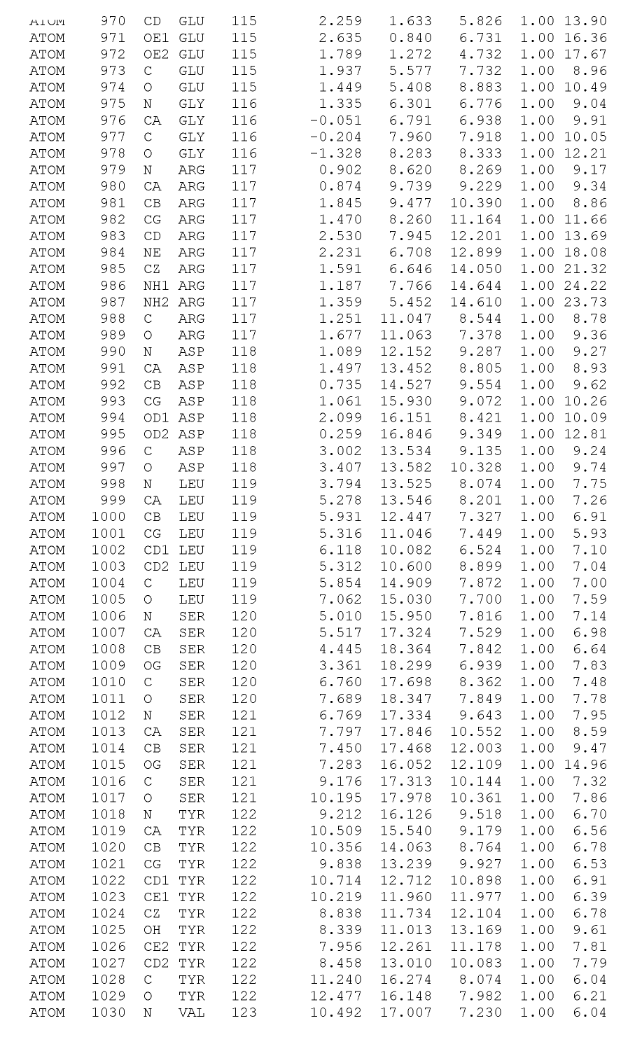

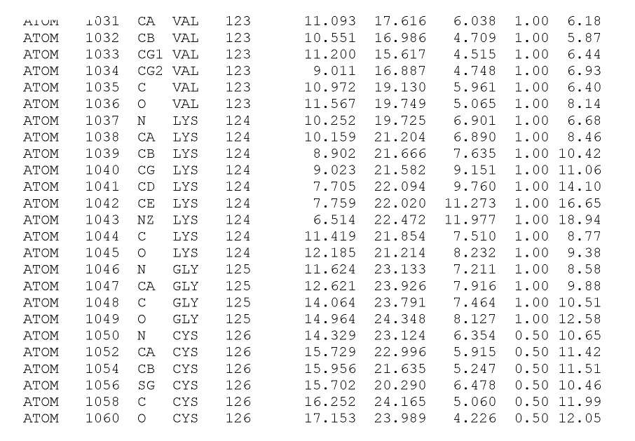

The crystal structure of the Opisthocomus hoazin lysozyme was solved at a resolution of 1.57 Å. The atomic coordinates of this structure are shown in FIG. 1. These atomic coordinates can be used to generate a three dimensional model depicting the structure of the Opisthocomus hoazin lysozyme or homologous structures (such as the variants of the present invention).

The Opisthocomus hoazin lysozyme belongs to the GH22 hydrolase family assigned E.C: number 3.2.1.17. The catalytic mechanism is believed to be the classical Koshland retaining one, with net retention configuration. This is often achieved by a two step, double displacement mechanism involving a covalent glycosyl-enzyme intermediate. Each step passes through an oxocarbenium ion-like transition state. The reaction occurs with acid/base and nucleophilic assistance provided by two amino acids side chains. In the first step (often called the glycosylation step), one amino acid residue (D51) plays the role of a nucleophile attacking the anomeric centre to displace the aglycon and form a glycosyl enzyme intermediate. At the same time, the second amino acid residue (E35) functions as an acid catalyst and protonates the glycosidic oxygen as the bond cleaves. In this second step (often called the deglycosylation step), the glcosyl is hydrolyzed by water, with the second amino acid residue (E35) now acting as a base catalyst deprotonating, the water molecule as it attacks. The pKa value of the acid/base group cycles between high and low values during catalysis to optimize for its role at each step of the catalysis.

Amino acid residues E35 and D51 (using SEQ ID NO:5 for numbering) have been identified as catalytic residues. In embodiments of the invention, (in addition to one or more of the alterations recited herein) the amino acid sequence of the lysozyme variants of the present invention no alteration is made for the amino acids corresponding to D51 and E35, using SEQ ID NO:5 for numbering.

Six substrate binding sites (A-F) for sugar molecules have also been indentified and the following residues/regions have been identified as involved in substrate binding at each of these six sites using SEQ ID NO:5 for numbering:

### D100 (Binding Site A);

### D100 (Binding Site B);

### N58, Y61, W62, P106 (Binding Site C);

### Q56, Y108, D51 (Binding Site D);

### N44, Q56, E35 (Binding Site E); and

### H34, D37, H113 (Binding Site F).

In an embodiment, (in addition to one or more of the alterations recited herein) the amino acid sequence of the lysozyme variant of the present invention has one or more of the following amino acids (using SEQ ID NO:5 for numbering) in the binding sites:

(A and B): D100 (no change);
(C′): 61Y or 61W and no change in N58 and P106, or specifically Y61W;
(D′): 108Y or 108V and no change in Q56 and D51, or specifically Y108V;
(E′): no change in N44, Q56, and E35; and/or
(F′): 34H or 34W, 37D or 37N, and no change in H113, or specifically H34W, D37N, and

### H34W+D37N.

In an embodiment, (in addition to one or more of the alterations recited herein), the amino acid sequence of the lysozyme variant of the present invention has no changes in the amino acids corresponding to E35, D51 and D100 (using SEQ ID NO:5 for numbering).

In an embodiment, (in addition to one or more of the alterations recited herein) the amino acid sequence of the lysozyme variant of the present invention has amino acids corresponding to

No changes in E35 and D51 in combination with binding site C′.

In an embodiment, (in addition to one or more of the alterations recited herein) the amino acid sequence of the lysozyme variant of the present invention has amino acids corresponding to

No changes in E35 and D51 in combination with binding site D′.

In an embodiment, (in addition to one or more of the alterations recited herein) the amino acid sequence of the lysozyme variant of the present invention has amino acids corresponding to

No changes in E35 and D51 in combination with binding site E′.

In an embodiment, (in addition to one or more of the alterations recited herein) the amino acid sequence of the lysozyme variant of the present invention has amino acids corresponding to

No changes in E35 and D51 in combination with binding site F′.

In an embodiment, (in addition to one or more of the alterations recited herein) the amino acid sequence of the lysozyme variant of the present invention has amino acids corresponding to

No changes in E35 and D51 in combination with 34H or 34W, 37D or 37N, 61Y or 61W, 108Y or 108V, and no change in N44, Q56, N58, W62, D100, P106, and H113 (using SEQ ID NO:5 for numbering).

### Altered Properties

In an embodiment, the present invention provides lysozyme variants having altered properties relative to the parent lysozyme or a reference lysozyme. Such altered properties include, but are not limited to, altered substrate specificity, altered pH-dependent activity profile, altered pH stability, altered pH optimum/activity, altered temperature stability, and/or altered resistance to non-enzymatic glycation.

In one embodiment, the isolated variant of the present invention comprises a combination of a substitution and insertion in the amino acid between position 46 to 49 (using SEQ ID NO:5 for numbering), and in particular, the two amino acid residues GP in positions 47 and 48 are changed into the three amino acids TDG (G47T+*47aD+P48G)

In one embodiment, the isolated variant of the present invention comprises or consists of an alteration at position 50 and/or position 90 (using SEQ ID NO:5 for numbering), and where the alteration broadens the pH-dependent activity profile of the lysozyme as compared to the lysozyme of SEQ ID NO:5 and/or to a parent or reference lysozyme.

In one embodiment, the isolated variant of the present invention comprises or consists of an alteration at one or more of positions selected from the group consisting of position 20, 32, 34, 47, 69, 74, 101, 109, and/or 124 (using SEQ ID NO:5 for numbering), and wherein the alteration improves the stability of the enzyme as compared to the lysozyme of SEQ ID NO:5 and/or to a parent or reference lysozyme.

Apart from the single alterations this means the following combinations:

20+32, 20+34, 20+47, 20+69, 20+74, 20+101, 20+109, 20+124, 32+34, 32+47, 32+69, 32+74, 32+101, 32+109, 32+124, 34+47, 34+69, 34+74, 34+101, 34+109, 34+124, 47+69, 47+74, 47+101, 47+109, 47+124, 69+74, 69+101, 69+109, 69+124, 74+101, 74+109, 74+124, 101+109, 101+124, 109+124, 20+32+34, 20+32+47, 20+32+69, 20+32+74, 20+32+101, 20+32+109, 20+32+124, 20+34+47, 20+34+69, 20+34+74, 20+34+101, 20+34+109, 20+34+124, 20+47+69, 20+47+74, 20+47+101, 20+47+109, 20+47+124, 20+69+74, 20+69+101, 20+69+109, 20+69+124, 20+74+101, 20+74+109, 20+74+124, 20+101+109, 20+101+124, 20+109+124, 32+34+47, 32+34+69, 32+34+74, 32+34+101, 32+34+109, 32+34+124, 32+47+69, 32+47+74, 32+47+101, 32+47+109, 32+47+124, 32+69+74, 32+69+101, 32+69+109, 32+69+124, 32+74+101, 32+74+109, 32+74+124, 32+101+109, 32+101+124, 32+109+124, 34+47+69, 34+47+74, 34+47+101, 34+47+109, 34+47+124, 34+69+74, 34+69+101, 34+69+109, 34+69+124, 34+74+101, 34+74+109, 34+74+124, 34+101+109, 34+101+124, 34+109+124, 47+69+74, 47+69+101, 47+69+109, 47+69+124, 47+74+101, 47+74+109, 47+74+124, 47+101+109, 47+101+124, 47+109+124, 69+74+101, 69+74+109, 69+74+124, 69+101+109, 69+101+124, 69+109+124, 74+101+109, 74+101+124, 74+109+124, 101+109+124, 20+32+34+47, 20+32+34+69, 20+32+34+74, 20+32+34+101, 20+32+34+109, 20+32+34+124, 20+32+47+69, 20+32+47+74, 20+32+47+101, 20+32+47+109, 20+32+47+124, 20+32+69+74, 20+32+69+101, 20+32+69+109, 20+32+69+124, 20+32+74+101, 20+32+74+109, 20+32+74+124, 20+32+101+109, 20+32+101+124, 20+32+109+124, 20+34+47+69, 20+34+47+74, 20+34+47+101, 20+34+47+109, 20+34+47+124, 20+34+69+74, 20+34+69+101, 20+34+69+109, 20+34+69+124, 20+34+74+101, 20+34+74+109, 20+34+74+124, 20+34+101+109, 20+34+101+124, 20+34+109+124, 20+47+69+74, 20+47+69+101, 20+47+69+109, 20+47+69+124, 20+47+74+101, 20+47+74+109, 20+47+74+124, 20+47+101+109, 20+47+101+124, 20+47+109+124, 20+69+74+101, 20+69+74+109, 20+69+74+124, 20+69+101+109, 20+69+101+124, 20+69+109+124, 20+74+101+109, 20+74+101+124, 20+74+109+124, 20+101+109+124, 32+34+47+69, 32+34+47+74, 32+34+47+101, 32+34+47+109, 32+34+47+124, 32+34+69+74, 32+34+69+101, 32+34+69+109, 32+34+69+124, 32+34+74+101, 32+34+74+109, 32+34+74+124, 32+34+101+109, 32+34+101+124, 32+34+109+124, 32+47+69+74, 32+47+69+101, 32+47+69+109, 32+47+69+124, 32+47+74+101, 32+47+74+109, 32+47+74+124, 32+47+101+109, 32+47+101+124, 32+47+109+124, 32+69+74+101, 32+69+74+109, 32+69+74+124, 32+69+101+109, 32+69+101+124, 32+69+109+124, 32+74+101+109, 32+74+101+124, 32+74+109+124, 32+101+109+124, 34+47+69+74, 34+47+69+101, 34+47+69+109, 34+47+69+124, 34+47+74+101, 34+47+74+109, 34+47+74+124, 34+47+101+109, 34+47+101+124, 34+47+109+124, 34+69+74+101, 34+69+74+109, 34+69+74+124, 34+69+101+109, 34+69+101+124, 34+69+109+124, 34+74+101+109, 34+74+101+124, 34+74+109+124, 34+101+109+124, 47+69+74+101, 47+69+74+109, 47+69+74+124, 47+69+101+109, 47+69+101+124, 47+69+109+124, 47+74+101+109, 47+74+101+124, 47+74+109+124, 47+101+109+124, 69+74+101+109, 69+74+101+124, 69+74+109+124, 69+101+109+124, 74+101+109+124, 20+32+34+47+69, 20+32+34+47+74, 20+32+34+47+101, 20+32+34+47+109, 20+32+34+47+124, 20+32+34+69+74, 20+32+34+69+101, 20+32+34+69+109, 20+32+34+69+124, 20+32+34+74+101, 20+32+34+74+109, 20+32+34+74+124, 20+32+34+101+109, 20+32+34+101+124, 20+32+34+109+124, 20+32+47+69+74, 20+32+47+69+101, 20+32+47+69+109, 20+32+47+69+124, 20+32+47+74+101, 20+32+47+74+109, 20+32+47+74+124, 20+32+47+101+109, 20+32+47+101+124, 20+32+47+109+124, 20+32+69+74+101, 20+32+69+74+109, 20+32+69+74+124, 20+32+69+101+109, 20+32+69+101+124, 20+32+69+109+124, 20+32+74+101+109, 20+32+74+101+124, 20+32+74+109+124, 20+32+101+109+124, 20+34+47+69+74, 20+34+47+69+101, 20+34+47+69+109, 20+34+47+69+124, 20+34+47+74+101, 20+34+47+74+109, 20+34+47+74+124, 20+34+47+101+109, 20+34+47+101+124, 20+34+47+109+124, 20+34+69+74+101, 20+34+69+74+109, 20+34+69+74+124, 20+34+69+101+109, 20+34+69+101+124, 20+34+69+109+124, 20+34+74+101+109, 20+34+74+101+124, 20+34+74+109+124, 20+34+101+109+124, 20+47+69+74+101, 20+47+69+74+109, 20+47+69+74+124, 20+47+69+101+109, 20+47+69+101+124, 20+47+69+109+124, 20+47+74+101+109, 20+47+74+101+124, 20+47+74+109+124, 20+47+101+109+124, 20+69+74+101+109, 20+69+74+101+124, 20+69+74+109+124, 20+69+101+109+124, 20+74+101+109+124, 32+34+47+69+74, 32+34+47+69+101, 32+34+47+69+109, 32+34+47+69+124, 32+34+47+74+101, 32+34+47+74+109, 32+34+47+74+124, 32+34+47+101+109, 32+34+47+101+124, 32+34+47+109+124, 32+34+69+74+101, 32+34+69+74+109, 32+34+69+74+124, 32+34+69+101+109, 32+34+69+101+124, 32+34+69+109+124, 32+34+74+101+109, 32+34+74+101+124, 32+34+74+109+124, 32+34+101+109+124, 32+47+69+74+101, 32+47+69+74+109, 32+47+69+74+124, 32+47+69+101+109, 32+47+69+101+124, 32+47+69+109+124, 32+47+74+101+109, 32+47+74+101+124, 32+47+74+109+124, 32+47+101+109+124, 32+69+74+101+109, 32+69+74+101+124, 32+69+74+109+124, 32+69+101+109+124, 32+74+101+109+124, 34+47+69+74+101, 34+47+69+74+109, 34+47+69+74+124, 34+47+69+101+109, 34+47+69+101+124, 34+47+69+109+124, 34+47+74+101+109, 34+47+74+101+124, 34+47+74+109+124, 34+47+101+109+124, 34+69+74+101+109, 34+69+74+101+124, 34+69+74+109+124, 34+69+101+109+124, 34+74+101+109+124, 47+69+74+101+109, 47+69+74+101+124, 47+69+74+109+124, 47+69+101+109+124, 47+74+101+109+124, 69+74+101+109+124, 20+32+34+47+69+74, 20+32+34+47+69+101, 20+32+34+47+69+109, 20+32+34+47+69+124, 20+32+34+47+74+101, 20+32+34+47+74+109, 20+32+34+47+74+124, 20+32+34+47+101+109, 20+32+34+47+101+124, 20+32+34+47+109+124, 20+32+34+69+74+101, 20+32+34+69+74+109, 20+32+34+69+74+124, 20+32+34+69+101+109, 20+32+34+69+101+124, 20+32+34+69+109+124, 20+32+34+74+101+109, 20+32+34+74+101+124, 20+32+34+74+109+124, 20+32+34+101+109+124, 20+32+47+69+74+101, 20+32+47+69+74+109, 20+32+47+69+74+124, 20+32+47+69+101+109, 20+32+47+69+101+124, 20+32+47+69+109+124, 20+32+47+74+101+109, 20+32+47+74+101+124, 20+32+47+74+109+124, 20+32+47+101+109+124, 20+32+69+74+101+109, 20+32+69+74+101+124, 20+32+69+74+109+124, 20+32+69+101+109+124, 20+32+74+101+109+124, 20+34+47+69+74+101, 20+34+47+69+74+109, 20+34+47+69+74+124, 20+34+47+69+101+109, 20+34+47+69+101+124, 20+34+47+69+109+124, 20+34+47+74+101+109, 20+34+47+74+101+124, 20+34+47+74+109+124, 20+34+47+101+109+124, 20+34+69+74+101+109, 20+34+69+74+101+124, 20+34+69+74+109+124, 20+34+69+101+109+124, 20+34+74+101+109+124, 20+47+69+74+101+109, 20+47+69+74+101+124, 20+47+69+74+109+124, 20+47+69+101+109+124, 20+47+74+101+109+124, 20+69+74+101+109+124, 32+34+47+69+74+101, 32+34+47+69+74+109, 32+34+47+69+74+124, 32+34+47+69+101+109, 32+34+47+69+101+124, 32+34+47+69+109+124, 32+34+47+74+101+109, 32+34+47+74+101+124, 32+34+47+74+109+124, 32+34+47+101+109+124, 32+34+69+74+101+109, 32+34+69+74+101+124, 32+34+69+74+109+124, 32+34+69+101+109+124, 32+34+74+101+109+124, 32+47+69+74+101+109, 32+47+69+74+101+124, 32+47+69+74+109+124, 32+47+69+101+109+124, 32+47+74+101+109+124, 32+69+74+101+109+124, 34+47+69+74+101+109, 34+47+69+74+101+124, 34+47+69+74+109+124, 34+47+69+101+109+124, 34+47+74+101+109+124, 34+69+74+101+109+124, 47+69+74+101+109+124, 20+32+34+47+69+74+101, 20+32+34+47+69+74+109, 20+32+34+47+69+74+124, 20+32+34+47+69+101+109, 20+32+34+47+69+101+124, 20+32+34+47+69+109+124, 20+32+34+47+74+101+109, 20+32+34+47+74+101+124, 20+32+34+47+74+109+124, 20+32+34+47+101+109+124, 20+32+34+69+74+101+109, 20+32+34+69+74+101+124, 20+32+34+69+74+109+124, 20+32+34+69+101+109+124, 20+32+34+74+101+109+124, 20+32+47+69+74+101+109, 20+32+47+69+74+101+124, 20+32+47+69+74+109+124, 20+32+47+69+101+109+124, 20+32+47+74+101+109+124, 20+32+69+74+101+109+124, 20+34+47+69+74+101+109, 20+34+47+69+74+101+124, 20+34+47+69+74+109+124, 20+34+47+69+101+109+124, 20+34+47+74+101+109+124, 20+34+69+74+101+109+124, 20+47+69+74+101+109+124, 32+34+47+69+74+101+109, 32+34+47+69+74+101+124, 32+34+47+69+74+109+124, 32+34+47+69+101+109+124, 32+34+47+74+101+109+124, 32+34+69+74+101+109+124, 32+47+69+74+101+109+124, 34+47+69+74+101+109+124, 20+32+34+47+69+74+101+109, 20+32+34+47+69+74+101+124, 20+32+34+47+69+74+109+124, 20+32+34+47+69+101+109+124, 20+32+34+47+74+101+109+124, 20+32+34+69+74+101+109+124, 20+32+47+69+74+101+109+124, 20+34+47+69+74+101+109+124, 32+34+47+69+74+101+109+124 and 20+32+34+47+69+74+101+109+124.

In one embodiment, the isolated variant of the present invention comprises or consists of an alteration at one or more positions selected from the group consisting of position 37, 41, 73, 81, 88, and/or 89 (using SEQ ID NO:5 for numbering), and wherein the alteration improves the stability of the enzyme at alkaline pH as compared to the lysozyme of SEQ ID NO:5 and/or to a parent or reference lysozyme.

Apart from the single alterations this means the following combinations:

37+41, 37+73, 37+81, 37+88, 37+89, 41+73, 41+81, 41+88, 41+89, 73+81, 73+88, 73+89, 81+88, 81+89, 88+89, 37+41+73, 37+41+81, 37+41+88, 37+41+89, 37+73+81, 37+73+88, 37+73+89, 37+81+88, 37+81+89, 37+88+89, 41+73+81, 41+73+88, 41+73+89, 41+81+88, 41+81+89, 41+88+89, 73+81+88, 73+81+89, 73+88+89, 81+88+89, 37+41+73+81, 37+41+73+88, 37+41+73+89, 37+41+81+88, 37+41+81+89, 37+41+88+89, 37+73+81+88, 37+73+81+89, 37+73+88+89, 37+81+88+89, 41+73+81+88, 41+73+81+89, 41+73+88+89, 41+81+88+89, 73+81+88+89, 37+41+73+81+88, 37+41+73+81+89, 37+41+73+88+89, 37+41+81+88+89, 37+73+81+88+89, 41+73+81+88+89 and 37+41+73+81+88+89.

In one embodiment, the isolated variant of the present invention comprises or consists of an alteration at one or more positions selected from the group consisting of position 10, 60, 67, 92, 96, 111 and/or 124 (using SEQ ID NO:5 for numbering), and wherein the alteration reduces the glycation susceptibility of the lysozyme as compared to the lysozyme of SEQ ID NO:5 and/or to a parent or reference lysozyme.

Apart from the single alterations this means the following combinations:

10+60, 10+67, 10+92, 10+96, 10+111, 10+124, 60+67, 60+92, 60+96, 60+111, 60+124, 67+92, 67+96, 67+111, 67+124, 92+96, 92+111, 92+124, 96+111, 96+124, 111+124, 10+60+67, 10+60+92, 10+60+96, 10+60+111, 10+60+124, 10+67+92, 10+67+96, 10+67+111, 10+67+124, 10+92+96, 10+92+111, 10+92+124, 10+96+111, 10+96+124, 10+111+124, 60+67+92, 60+67+96, 60+67+111, 60+67+124, 60+92+96, 60+92+111, 60+92+124, 60+96+111, 60+96+124, 60+111+124, 67+92+96, 67+92+111, 67+92+124, 67+96+111, 67+96+124, 67+111+124, 92+96+111, 92+96+124, 92+111+124, 96+111+124, 10+60+67+92, 10+60+67+96, 10+60+67+111, 10+60+67+124, 10+60+92+96, 10+60+92+111, 10+60+92+124, 10+60+96+111, 10+60+96+124, 10+60+111+124, 10+67+92+96, 10+67+92+111, 10+67+92+124, 10+67+96+111, 10+67+96+124, 10+67+111+124, 10+92+96+111, 10+92+96+124, 10+92+111+124, 10+96+111+124, 60+67+92+96, 60+67+92+111, 60+67+92+124, 60+67+96+111, 60+67+96+124, 60+67+111+124, 60+92+96+111, 60+92+96+124, 60+92+111+124, 60+96+111+124, 67+92+96+111, 67+92+96+124, 67+92+111+124, 67+96+111+124, 92+96+111+124, 10+60+67+92+96, 10+60+67+92+111, 10+60+67+92+124, 10+60+67+96+111, 10+60+67+96+124, 10+60+67+111+124, 10+60+92+96+111, 10+60+92+96+124, 10+60+92+111+124, 10+60+96+111+124, 10+67+92+96+111, 10+67+92+96+124, 10+67+92+111+124, 10+67+96+111+124, 10+92+96+111+124, 60+67+92+96+111, 60+67+92+96+124, 60+67+92+111+124, 60+67+96+111+124, 60+92+96+111+124, 67+92+96+111+124, 10+60+67+92+96+111, 10+60+67+92+96+124, 10+60+67+92+111+124, 10+60+67+96+111+124, 10+60+92+96+111+124, 10+67+92+96+111+124, 60+67+92+96+111+124 and 10+60+67+92+96+111+124.

In one embodiment, the isolated variant of the present invention comprises or consists of an alteration at one or more positions selected from the group consisting of position 61, 97, 108 and/or 109 (using SEQ ID NO:5 for numbering), and wherein the alterations improves the substrate specificity of the lysozyme as compared to the lysozyme of SEQ ID NO:5 and/or to a parent or reference lysozyme.

Apart from the single alterations this means the following combinations:

61+97, 61+108, 61+109, 97+108, 97+109, 108+109, 61+97+108, 61+97+109, 61+108+109, 97+108+109 and 61+97+108+109.

In another aspect, the lysozyme variants of the present invention comprise or consist of alterations at position selected from the group consisting of 9, 25 and 29 (using SEQ ID NO:5 for numbering). In an embodiment, the alterations at positions 9, 25 and/or 29 improve the stability of the enzyme as compared to the lysozyme of SEQ ID NO:5 and/or to a parent or reference lysozyme. In specific embodiments, the lysozyme variants of the present invention comprise or consist of the alterations V9A, I25L and I29L (using SEQ ID NO:5 for numbering).

Apart from the single alterations this means the following combinations:

9+25, 9+29, 25+29 and 9+25+29, where specific positions in the combinations are modified as indicated.

In another aspect, the lysozyme variants of the present invention comprise or consist of alterations at position 3, 11, 17, 87, 91 and 124 (using SEQ ID NO:5 for numbering). In an embodiment, the alterations at positions 3, 11, 17, 87, 91 and 124 improve the stability of the enzyme as compared to the lysozyme of SEQ ID NO:5 and/or to a parent or reference lysozyme. In an embodiment, the lysozyme variants of the present invention comprise or consist of the alterations (using SEQ ID NO:5 for numbering).

Apart from the single alterations this means the following combinations:

3+11, 3+17, 3+87, 3+91, 3+124, 11+17, 11+87, 11+91, 11+124, 17+87, 17+91, 17+124, 87+91, 87+124, 91+124, 3+11+17, 3+11+87, 3+11+91, 3+11+124, 3+17+87, 3+17+91, 3+17+124, 3+87+91, 3+87+124, 3+91+124, 11+17+87, 11+17+91, 11+17+124, 11+87+91, 11+87+124, 11+91+124, 17+87+91, 17+87+124, 17+91+124, 87+91+124, 3+11+17+87, 3+11+17+91, 3+11+17+124, 3+11+87+91, 3+11+87+124, 3+11+91+124, 3+17+87+91, 3+17+87+124, 3+17+91+124, 3+87+91+124, 11+17+87+91, 11+17+87+124, 11+17+91+124, 11+87+91+124, 17+87+91+124, 3+11+17+87+91, 3+11+17+87+124, 3+11+17+91+124, 3+11+87+91+124, 3+17+87+91+124, 11+17+87+91+124 and 3+11+17+87+91+124.

In another aspect, the lysozyme variants of the present invention comprise or consist of alterations at position 98 and 104 (using SEQ ID NO:5 for numbering). In an embodiment, the alterations at positions 98 and 104 improve the stability of the enzyme as compared to the lysozyme of SEQ ID NO:5 and/or to a parent or reference lysozyme. In an embodiment, the lysozyme variants of the present invention comprise or consist of the alterations A98V and L104I and the combination A98V+L104I (using SEQ ID NO:5 for numbering).

In another aspect, the lysozyme variants of the present invention comprise or consist of alterations at position 3, 9, 11, 17, 25, 29, 87, 91, 98 and 104 (using SEQ ID NO:5 for numbering). In an embodiment, the alterations at positions 3, 9, 11, 17, 25, 29, 87, 91, 98 and 104 improve the stability of the enzyme as compared to the lysozyme of SEQ ID NO:5 and/or to a parent or reference lysozyme. In an embodiment, the lysozyme variants of the present invention comprise or consist of the alterations 13F, V9A, 111T, F17L, I25L, I29L L87I, I91V, V98A, and L104I (using SEQ ID NO:5 for numbering).

Apart from the single alterations this means the following combinations:

3+9, 3+11, 3+17, 3+25, 3+29, 3+87, 3+91, 3+98, 3+104, 9+11, 9+17, 9+25, 9+29, 9+87, 9+91, 9+98, 9+104, 11+17, 11+25, 11+29, 11+87, 11+91, 11+98, 11+104, 17+25, 17+29, 17+87, 17+91, 17+98, 17+104, 25+29, 25+87, 25+91, 25+98, 25+104, 29+87, 29+91, 29+98, 29+104, 87+91, 87+98, 87+104, 91+98, 91+104, 98+104, 3+9+11, 3+9+17, 3+9+25, 3+9+29, 3+9+87, 3+9+91, 3+9+98, 3+9+104, 3+11+17, 3+11+25, 3+11+29, 3+11+87, 3+11+91, 3+11+98, 3+11+104, 3+17+25, 3+17+29, 3+17+87, 3+17+91, 3+17+98, 3+17+104, 3+25+29, 3+25+87, 3+25+91, 3+25+98, 3+25+104, 3+29+87, 3+29+91, 3+29+98, 3+29+104, 3+87+91, 3+87+98, 3+87+104, 3+91+98, 3+91+104, 3+98+104, 9+11+17, 9+11+25, 9+11+29, 9+11+87, 9+11+91, 9+11+98, 9+11+104, 9+17+25, 9+17+29, 9+17+87, 9+17+91, 9+17+98, 9+17+104, 9+25+29, 9+25+87, 9+25+91, 9+25+98, 9+25+104, 9+29+87, 9+29+91, 9+29+98, 9+29+104, 9+87+91, 9+87+98, 9+87+104, 9+91+98, 9+91+104, 9+98+104, 11+17+25, 11+17+29, 11+17+87, 11+17+91, 11+17+98, 11+17+104, 11+25+29, 11+25+87, 11+25+91, 11+25+98, 11+25+104, 11+29+87, 11+29+91, 11+29+98, 11+29+104, 11+87+91, 11+87+98, 11+87+104, 11+91+98, 11+91+104, 11+98+104, 17+25+29, 17+25+87, 17+25+91, 17+25+98, 17+25+104, 17+29+87, 17+29+91, 17+29+98, 17+29+104, 17+87+91, 17+87+98, 17+87+104, 17+91+98, 17+91+104, 17+98+104, 25+29+87, 25+29+91, 25+29+98, 25+29+104, 25+87+91, 25+87+98, 25+87+104, 25+91+98, 25+91+104, 25+98+104, 29+87+91, 29+87+98, 29+87+104, 29+91+98, 29+91+104, 29+98+104, 87+91+98, 87+91+104, 87+98+104, 91+98+104, 3+9+11+17, 3+9+11+25, 3+9+11+29, 3+9+11+87, 3+9+11+91, 3+9+11+98, 3+9+11+104, 3+9+17+25, 3+9+17+29, 3+9+17+87, 3+9+17+91, 3+9+17+98, 3+9+17+104, 3+9+25+29, 3+9+25+87, 3+9+25+91, 3+9+25+98, 3+9+25+104, 3+9+29+87, 3+9+29+91, 3+9+29+98, 3+9+29+104, 3+9+87+91, 3+9+87+98, 3+9+87+104, 3+9+91+98, 3+9+91+104, 3+9+98+104, 3+11+17+25, 3+11+17+29, 3+11+17+87, 3+11+17+91, 3+11+17+98, 3+11+17+104, 3+11+25+29, 3+11+25+87, 3+11+25+91, 3+11+25+98, 3+11+25+104, 3+11+29+87, 3+11+29+91, 3+11+29+98, 3+11+29+104, 3+11+87+91, 3+11+87+98, 3+11+87+104, 3+11+91+98, 3+11+91+104, 3+11+98+104, 3+17+25+29, 3+17+25+87, 3+17+25+91, 3+17+25+98, 3+17+25+104, 3+17+29+87, 3+17+29+91, 3+17+29+98, 3+17+29+104, 3+17+87+91, 3+17+87+98, 3+17+87+104, 3+17+91+98, 3+17+91+104, 3+17+98+104, 3+25+29+87, 3+25+29+91, 3+25+29+98, 3+25+29+104, 3+25+87+91, 3+25+87+98, 3+25+87+104, 3+25+91+98, 3+25+91+104, 3+25+98+104, 3+29+87+91, 3+29+87+98, 3+29+87+104, 3+29+91+98, 3+29+91+104, 3+29+98+104, 3+87+91+98, 3+87+91+104, 3+87+98+104, 3+91+98+104, 9+11+17+25, 9+11+17+29, 9+11+17+87, 9+11+17+91, 9+11+17+98, 9+11+17+104, 9+11+25+29, 9+11+25+87, 9+11+25+91, 9+11+25+98, 9+11+25+104, 9+11+29+87, 9+11+29+91, 9+11+29+98, 9+11+29+104, 9+11+87+91, 9+11+87+98, 9+11+87+104, 9+11+91+98, 9+11+91+104, 9+11+98+104, 9+17+25+29, 9+17+25+87, 9+17+25+91, 9+17+25+98, 9+17+25+104, 9+17+29+87, 9+17+29+91, 9+17+29+98, 9+17+29+104, 9+17+87+91, 9+17+87+98, 9+17+87+104, 9+17+91+98, 9+17+91+104, 9+17+98+104, 9+25+29+87, 9+25+29+91, 9+25+29+98, 9+25+29+104, 9+25+87+91, 9+25+87+98, 9+25+87+104, 9+25+91+98, 9+25+91+104, 9+25+98+104, 9+29+87+91, 9+29+87+98, 9+29+87+104, 9+29+91+98, 9+29+91+104, 9+29+98+104, 9+87+91+98, 9+87+91+104, 9+87+98+104, 9+91+98+104, 11+17+25+29, 11+17+25+87, 11+17+25+91, 11+17+25+98, 11+17+25+104, 11+17+29+87, 11+17+29+91, 11+17+29+98, 11+17+29+104, 11+17+87+91, 11+17+87+98, 11+17+87+104, 11+17+91+98, 11+17+91+104, 11+17+98+104, 11+25+29+87, 11+25+29+91, 11+25+29+98, 11+25+29+104, 11+25+87+91, 11+25+87+98, 11+25+87+104, 11+25+91+98, 11+25+91+104, 11+25+98+104, 11+29+87+91, 11+29+87+98, 11+29+87+104, 11+29+91+98, 11+29+91+104, 11+29+98+104, 11+87+91+98, 11+87+91+104, 11+87+98+104, 11+91+98+104, 17+25+29+87, 17+25+29+91, 17+25+29+98, 17+25+29+104, 17+25+87+91, 17+25+87+98, 17+25+87+104, 17+25+91+98, 17+25+91+104, 17+25+98+104, 17+29+87+91, 17+29+87+98, 17+29+87+104, 17+29+91+98, 17+29+91+104, 17+29+98+104, 17+87+91+98, 17+87+91+104, 17+87+98+104, 17+91+98+104, 25+29+87+91, 25+29+87+98, 25+29+87+104, 25+29+91+98, 25+29+91+104, 25+29+98+104, 25+87+91+98, 25+87+91+104, 25+87+98+104, 25+91+98+104, 29+87+91+98, 29+87+91+104, 29+87+98+104, 29+91+98+104, 87+91+98+104, 3+9+11+17+25, 3+9+11+17+29, 3+9+11+17+87, 3+9+11+17+91, 3+9+11+17+98, 3+9+11+17+104, 3+9+11+25+29, 3+9+11+25+87, 3+9+11+25+91, 3+9+11+25+98, 3+9+11+25+104, 3+9+11+29+87, 3+9+11+29+91, 3+9+11+29+98, 3+9+11+29+104, 3+9+11+87+91, 3+9+11+87+98, 3+9+11+87+104, 3+9+11+91+98, 3+9+11+91+104, 3+9+11+98+104, 3+9+17+25+29, 3+9+17+25+87, 3+9+17+25+91, 3+9+17+25+98, 3+9+17+25+104, 3+9+17+29+87, 3+9+17+29+91, 3+9+17+29+98, 3+9+17+29+104, 3+9+17+87+91, 3+9+17+87+98, 3+9+17+87+104, 3+9+17+91+98, 3+9+17+91+104, 3+9+17+98+104, 3+9+25+29+87, 3+9+25+29+91, 3+9+25+29+98, 3+9+25+29+104, 3+9+25+87+91, 3+9+25+87+98, 3+9+25+87+104, 3+9+25+91+98, 3+9+25+91+104, 3+9+25+98+104, 3+9+29+87+91, 3+9+29+87+98, 3+9+29+87+104, 3+9+29+91+98, 3+9+29+91+104, 3+9+29+98+104, 3+9+87+91+98, 3+9+87+91+104, 3+9+87+98+104, 3+9+91+98+104, 3+11+17+25+29, 3+11+17+25+87, 3+11+17+25+91, 3+11+17+25+98, 3+11+17+25+104, 3+11+17+29+87, 3+11+17+29+91, 3+11+17+29+98, 3+11+17+29+104, 3+11+17+87+91, 3+11+17+87+98, 3+11+17+87+104, 3+11+17+91+98, 3+11+17+91+104, 3+11+17+98+104, 3+11+25+29+87, 3+11+25+29+91, 3+11+25+29+98, 3+11+25+29+104, 3+11+25+87+91, 3+11+25+87+98, 3+11+25+87+104, 3+11+25+91+98, 3+11+25+91+104, 3+11+25+98+104, 3+11+29+87+91, 3+11+29+87+98, 3+11+29+87+104, 3+11+29+91+98, 3+11+29+91+104, 3+11+29+98+104, 3+11+87+91+98, 3+11+87+91+104, 3+11+87+98+104, 3+11+91+98+104, 3+17+25+29+87, 3+17+25+29+91, 3+17+25+29+98, 3+17+25+29+104, 3+17+25+87+91, 3+17+25+87+98, 3+17+25+87+104, 3+17+25+91+98, 3+17+25+91+104, 3+17+25+98+104, 3+17+29+87+91, 3+17+29+87+98, 3+17+29+87+104, 3+17+29+91+98, 3+17+29+91+104, 3+17+29+98+104, 3+17+87+91+98, 3+17+87+91+104, 3+17+87+98+104, 3+17+91+98+104, 3+25+29+87+91, 3+25+29+87+98, 3+25+29+87+104, 3+25+29+91+98, 3+25+29+91+104, 3+25+29+98+104, 3+25+87+91+98, 3+25+87+91+104, 3+25+87+98+104, 3+25+91+98+104, 3+29+87+91+98, 3+29+87+91+104, 3+29+87+98+104, 3+29+91+98+104, 3+87+91+98+104, 9+11+17+25+29, 9+11+17+25+87, 9+11+17+25+91, 9+11+17+25+98, 9+11+17+25+104, 9+11+17+29+87, 9+11+17+29+91, 9+11+17+29+98, 9+11+17+29+104, 9+11+17+87+91, 9+11+17+87+98, 9+11+17+87+104, 9+11+17+91+98, 9+11+17+91+104, 9+11+17+98+104, 9+11+25+29+87, 9+11+25+29+91, 9+11+25+29+98, 9+11+25+29+104, 9+11+25+87+91, 9+11+25+87+98, 9+11+25+87+104, 9+11+25+91+98, 9+11+25+91+104, 9+11+25+98+104, 9+11+29+87+91, 9+11+29+87+98, 9+11+29+87+104, 9+11+29+91+98, 9+11+29+91+104, 9+11+29+98+104, 9+11+87+91+98, 9+11+87+91+104, 9+11+87+98+104, 9+11+91+98+104, 9+17+25+29+87, 9+17+25+29+91, 9+17+25+29+98, 9+17+25+29+104, 9+17+25+87+91, 9+17+25+87+98, 9+17+25+87+104, 9+17+25+91+98, 9+17+25+91+104, 9+17+25+98+104, 9+17+29+87+91, 9+17+29+87+98, 9+17+29+87+104, 9+17+29+91+98, 9+17+29+91+104, 9+17+29+98+104, 9+17+87+91+98, 9+17+87+91+104, 9+17+87+98+104, 9+17+91+98+104, 9+25+29+87+91, 9+25+29+87+98, 9+25+29+87+104, 9+25+29+91+98, 9+25+29+91+104, 9+25+29+98+104, 9+25+87+91+98, 9+25+87+91+104, 9+25+87+98+104, 9+25+91+98+104, 9+29+87+91+98, 9+29+87+91+104, 9+29+87+98+104, 9+29+91+98+104, 9+87+91+98+104, 11+17+25+29+87, 11+17+25+29+91, 11+17+25+29+98, 11+17+25+29+104, 11+17+25+87+91, 11+17+25+87+98, 11+17+25+87+104, 11+17+25+91+98, 11+17+25+91+104, 11+17+25+98+104, 11+17+29+87+91, 11+17+29+87+98, 11+17+29+87+104, 11+17+29+91+98, 11+17+29+91+104, 11+17+29+98+104, 11+17+87+91+98, 11+17+87+91+104, 11+17+87+98+104, 11+17+91+98+104, 11+25+29+87+91, 11+25+29+87+98, 11+25+29+87+104, 11+25+29+91+98, 11+25+29+91+104, 11+25+29+98+104, 11+25+87+91+98, 11+25+87+91+104, 11+25+87+98+104, 11+25+91+98+104, 11+29+87+91+98, 11+29+87+91+104, 11+29+87+98+104, 11+29+91+98+104, 11+87+91+98+104, 17+25+29+87+91, 17+25+29+87+98, 17+25+29+87+104, 17+25+29+91+98, 17+25+29+91+104, 17+25+29+98+104, 17+25+87+91+98, 17+25+87+91+104, 17+25+87+98+104, 17+25+91+98+104, 17+29+87+91+98, 17+29+87+91+104, 17+29+87+98+104, 17+29+91+98+104, 17+87+91+98+104, 25+29+87+91+98, 25+29+87+91+104, 25+29+87+98+104, 25+29+91+98+104, 25+87+91+98+104, 29+87+91+98+104, 3+9+11+17+25+29, 3+9+11+17+25+87, 3+9+11+17+25+91, 3+9+11+17+25+98, 3+9+11+17+25+104, 3+9+11+17+29+87, 3+9+11+17+29+91, 3+9+11+17+29+98, 3+9+11+17+29+104, 3+9+11+17+87+91, 3+9+11+17+87+98, 3+9+11+17+87+104, 3+9+11+17+91+98, 3+9+11+17+91+104, 3+9+11+17+98+104, 3+9+11+25+29+87, 3+9+11+25+29+91, 3+9+11+25+29+98, 3+9+11+25+29+104, 3+9+11+25+87+91, 3+9+11+25+87+98, 3+9+11+25+87+104, 3+9+11+25+91+98, 3+9+11+25+91+104, 3+9+11+25+98+104, 3+9+11+29+87+91, 3+9+11+29+87+98, 3+9+11+29+87+104, 3+9+11+29+91+98, 3+9+11+29+91+104, 3+9+11+29+98+104, 3+9+11+87+91+98, 3+9+11+87+91+104, 3+9+11+87+98+104, 3+9+11+91+98+104, 3+9+17+25+29+87, 3+9+17+25+29+91, 3+9+17+25+29+98, 3+9+17+25+29+104, 3+9+17+25+87+91, 3+9+17+25+87+98, 3+9+17+25+87+104, 3+9+17+25+91+98, 3+9+17+25+91+104, 3+9+17+25+98+104, 3+9+17+29+87+91, 3+9+17+29+87+98, 3+9+17+29+87+104, 3+9+17+29+91+98, 3+9+17+29+91+104, 3+9+17+29+98+104, 3+9+17+87+91+98, 3+9+17+87+91+104, 3+9+17+87+98+104, 3+9+17+91+98+104, 3+9+25+29+87+91, 3+9+25+29+87+98, 3+9+25+29+87+104, 3+9+25+29+91+98, 3+9+25+29+91+104, 3+9+25+29+98+104, 3+9+25+87+91+98, 3+9+25+87+91+104, 3+9+25+87+98+104, 3+9+25+91+98+104, 3+9+29+87+91+98, 3+9+29+87+91+104, 3+9+29+87+98+104, 3+9+29+91+98+104, 3+9+87+91+98+104, 3+11+17+25+29+87, 3+11+17+25+29+91, 3+11+17+25+29+98, 3+11+17+25+29+104, 3+11+17+25+87+91, 3+11+17+25+87+98, 3+11+17+25+87+104, 3+11+17+25+91+98, 3+11+17+25+91+104, 3+11+17+25+98+104, 3+11+17+29+87+91, 3+11+17+29+87+98, 3+11+17+29+87+104, 3+11+17+29+91+98, 3+11+17+29+91+104, 3+11+17+29+98+104, 3+11+17+87+91+98, 3+11+17+87+91+104, 3+11+17+87+98+104, 3+11+17+91+98+104, 3+11+25+29+87+91, 3+11+25+29+87+98, 3+11+25+29+87+104, 3+11+25+29+91+98, 3+11+25+29+91+104, 3+11+25+29+98+104, 3+11+25+87+91+98, 3+11+25+87+91+104, 3+11+25+87+98+104, 3+11+25+91+98+104, 3+11+29+87+91+98, 3+11+29+87+91+104, 3+11+29+87+98+104, 3+11+29+91+98+104, 3+11+87+91+98+104, 3+17+25+29+87+91, 3+17+25+29+87+98, 3+17+25+29+87+104, 3+17+25+29+91+98, 3+17+25+29+91+104, 3+17+25+29+98+104, 3+17+25+87+91+98, 3+17+25+87+91+104, 3+17+25+87+98+104, 3+17+25+91+98+104, 3+17+29+87+91+98, 3+17+29+87+91+104, 3+17+29+87+98+104, 3+17+29+91+98+104, 3+17+87+91+98+104, 3+25+29+87+91+98, 3+25+29+87+91+104, 3+25+29+87+98+104, 3+25+29+91+98+104, 3+25+87+91+98+104, 3+29+87+91+98+104, 9+11+17+25+29+87, 9+11+17+25+29+91, 9+11+17+25+29+98, 9+11+17+25+29+104, 9+11+17+25+87+91, 9+11+17+25+87+98, 9+11+17+25+87+104, 9+11+17+25+91+98, 9+11+17+25+91+104, 9+11+17+25+98+104, 9+11+17+29+87+91, 9+11+17+29+87+98, 9+11+17+29+87+104, 9+11+17+29+91+98, 9+11+17+29+91+104, 9+11+17+29+98+104, 9+11+17+87+91+98, 9+11+17+87+91+104, 9+11+17+87+98+104, 9+11+17+91+98+104, 9+11+25+29+87+91, 9+11+25+29+87+98, 9+11+25+29+87+104, 9+11+25+29+91+98, 9+11+25+29+91+104, 9+11+25+29+98+104, 9+11+25+87+91+98, 9+11+25+87+91+104, 9+11+25+87+98+104, 9+11+25+91+98+104, 9+11+29+87+91+98, 9+11+29+87+91+104, 9+11+29+87+98+104, 9+11+29+91+98+104, 9+11+87+91+98+104, 9+17+25+29+87+91, 9+17+25+29+87+98, 9+17+25+29+87+104, 9+17+25+29+91+98, 9+17+25+29+91+104, 9+17+25+29+98+104, 9+17+25+87+91+98, 9+17+25+87+91+104, 9+17+25+87+98+104, 9+17+25+91+98+104, 9+17+29+87+91+98, 9+17+29+87+91+104, 9+17+29+87+98+104, 9+17+29+91+98+104, 9+17+87+91+98+104, 9+25+29+87+91+98, 9+25+29+87+91+104, 9+25+29+87+98+104, 9+25+29+91+98+104, 9+25+87+91+98+104, 9+29+87+91+98+104, 11+17+25+29+87+91, 11+17+25+29+87+98, 11+17+25+29+87+104, 11+17+25+29+91+98, 11+17+25+29+91+104, 11+17+25+29+98+104, 11+17+25+87+91+98, 11+17+25+87+91+104, 11+17+25+87+98+104, 11+17+25+91+98+104, 11+17+29+87+91+98, 11+17+29+87+91+104, 11+17+29+87+98+104, 11+17+29+91+98+104, 11+17+87+91+98+104, 11+25+29+87+91+98, 11+25+29+87+91+104, 11+25+29+87+98+104, 11+25+29+91+98+104, 11+25+87+91+98+104, 11+29+87+91+98+104, 17+25+29+87+91+98, 17+25+29+87+91+104, 17+25+29+87+98+104, 17+25+29+91+98+104, 17+25+87+91+98+104, 17+29+87+91+98+104, 25+29+87+91+98+104, 3+9+11+17+25+29+87, 3+9+11+17+25+29+91, 3+9+11+17+25+29+98, 3+9+11+17+25+29+104, 3+9+11+17+25+87+91, 3+9+11+17+25+87+98, 3+9+11+17+25+87+104, 3+9+11+17+25+91+98, 3+9+11+17+25+91+104, 3+9+11+17+25+98+104, 3+9+11+17+29+87+91, 3+9+11+17+29+87+98, 3+9+11+17+29+87+104, 3+9+11+17+29+91+98, 3+9+11+17+29+91+104, 3+9+11+17+29+98+104, 3+9+11+17+87+91+98, 3+9+11+17+87+91+104, 3+9+11+17+87+98+104, 3+9+11+17+91+98+104, 3+9+11+25+29+87+91, 3+9+11+25+29+87+98, 3+9+11+25+29+87+104, 3+9+11+25+29+91+98, 3+9+11+25+29+91+104, 3+9+11+25+29+98+104, 3+9+11+25+87+91+98, 3+9+11+25+87+91+104, 3+9+11+25+87+98+104, 3+9+11+25+91+98+104, 3+9+11+29+87+91+98, 3+9+11+29+87+91+104, 3+9+11+29+87+98+104, 3+9+11+29+91+98+104, 3+9+11+87+91+98+104, 3+9+17+25+29+87+91, 3+9+17+25+29+87+98, 3+9+17+25+29+87+104, 3+9+17+25+29+91+98, 3+9+17+25+29+91+104, 3+9+17+25+29+98+104, 3+9+17+25+87+91+98, 3+9+17+25+87+91+104, 3+9+17+25+87+98+104, 3+9+17+25+91+98+104, 3+9+17+29+87+91+98, 3+9+17+29+87+91+104, 3+9+17+29+87+98+104, 3+9+17+29+91+98+104, 3+9+17+87+91+98+104, 3+9+25+29+87+91+98, 3+9+25+29+87+91+104, 3+9+25+29+87+98+104, 3+9+25+29+91+98+104, 3+9+25+87+91+98+104, 3+9+29+87+91+98+104, 3+11+17+25+29+87+91, 3+11+17+25+29+87+98, 3+11+17+25+29+87+104, 3+11+17+25+29+91+98, 3+11+17+25+29+91+104, 3+11+17+25+29+98+104, 3+11+17+25+87+91+98, 3+11+17+25+87+91+104, 3+11+17+25+87+98+104, 3+11+17+25+91+98+104, 3+11+17+29+87+91+98, 3+11+17+29+87+91+104, 3+11+17+29+87+98+104, 3+11+17+29+91+98+104, 3+11+17+87+91+98+104, 3+11+25+29+87+91+98, 3+11+25+29+87+91+104, 3+11+25+29+87+98+104, 3+11+25+29+91+98+104, 3+11+25+87+91+98+104, 3+11+29+87+91+98+104, 3+17+25+29+87+91+98, 3+17+25+29+87+91+104, 3+17+25+29+87+98+104, 3+17+25+29+91+98+104, 3+17+25+87+91+98+104, 3+17+29+87+91+98+104, 3+25+29+87+91+98+104, 9+11+17+25+29+87+91, 9+11+17+25+29+87+98, 9+11+17+25+29+87+104, 9+11+17+25+29+91+98, 9+11+17+25+29+91+104, 9+11+17+25+29+98+104, 9+11+17+25+87+91+98, 9+11+17+25+87+91+104, 9+11+17+25+87+98+104, 9+11+17+25+91+98+104, 9+11+17+29+87+91+98, 9+11+17+29+87+91+104, 9+11+17+29+87+98+104, 9+11+17+29+91+98+104, 9+11+17+87+91+98+104, 9+11+25+29+87+91+98, 9+11+25+29+87+91+104, 9+11+25+29+87+98+104, 9+11+25+29+91+98+104, 9+11+25+87+91+98+104, 9+11+29+87+91+98+104, 9+17+25+29+87+91+98, 9+17+25+29+87+91+104, 9+17+25+29+87+98+104, 9+17+25+29+91+98+104, 9+17+25+87+91+98+104, 9+17+29+87+91+98+104, 9+25+29+87+91+98+104, 11+17+25+29+87+91+98, 11+17+25+29+87+91+104, 11+17+25+29+87+98+104, 11+17+25+29+91+98+104, 11+17+25+87+91+98+104, 11+17+29+87+91+98+104, 11+25+29+87+91+98+104, 17+25+29+87+91+98+104, 3+9+11+17+25+29+87+91, 3+9+11+17+25+29+87+98, 3+9+11+17+25+29+87+104, 3+9+11+17+25+29+91+98, 3+9+11+17+25+29+91+104, 3+9+11+17+25+29+98+104, 3+9+11+17+25+87+91+98, 3+9+11+17+25+87+91+104, 3+9+11+17+25+87+98+104, 3+9+11+17+25+91+98+104, 3+9+11+17+29+87+91+98, 3+9+11+17+29+87+91+104, 3+9+11+17+29+87+98+104, 3+9+11+17+29+91+98+104, 3+9+11+17+87+91+98+104, 3+9+11+25+29+87+91+98, 3+9+11+25+29+87+91+104, 3+9+11+25+29+87+98+104, 3+9+11+25+29+91+98+104, 3+9+11+25+87+91+98+104, 3+9+11+29+87+91+98+104, 3+9+17+25+29+87+91+98, 3+9+17+25+29+87+91+104, 3+9+17+25+29+87+98+104, 3+9+17+25+29+91+98+104, 3+9+17+25+87+91+98+104, 3+9+17+29+87+91+98+104, 3+9+25+29+87+91+98+104, 3+11+17+25+29+87+91+98, 3+11+17+25+29+87+91+104, 3+11+17+25+29+87+98+104, 3+11+17+25+29+91+98+104, 3+11+17+25+87+91+98+104, 3+11+17+29+87+91+98+104, 3+11+25+29+87+91+98+104, 3+17+25+29+87+91+98+104, 9+11+17+25+29+87+91+98, 9+11+17+25+29+87+91+104, 9+11+17+25+29+87+98+104, 9+11+17+25+29+91+98+104, 9+11+17+25+87+91+98+104, 9+11+17+29+87+91+98+104, 9+11+25+29+87+91+98+104, 9+17+25+29+87+91+98+104, 11+17+25+29+87+91+98+104, 3+9+11+17+25+29+87+91+98, 3+9+11+17+25+29+87+91+104, 3+9+11+17+25+29+87+98+104, 3+9+11+17+25+29+91+98+104, 3+9+11+17+25+87+91+98+104, 3+9+11+17+29+87+91+98+104, 3+9+11+25+29+87+91+98+104, 3+9+17+25+29+87+91+98+104, 3+11+17+25+29+87+91+98+104, 9+11+17+25+29+87+91+98+104 and 3+9+11+17+25+29+87+91+98+104, where specific positions in the combinations are modified as indicated.

In another aspect, the lysozyme variants of the present invention comprise or consist of alterations at position 47 and 48 and the combination 47+48 (using SEQ ID NO:5 for numbering).

It is understood that the embodiments described herein can be combined if desired, especially that the indicated positions and specific alterations may be combined and the positions and specific alterations may be applied in polypeptides having specified sequence identities.

### Polynucleotides

The present invention also relates to isolated polynucleotides that encode variants of a parent lysozyme according to the present invention. In particular polynucleotides that encode a lysozyme variant as described in the variant section above, is encompassed by the present invention. Polynucleotides of the invention will hybridize to a denatured double-stranded DNA probe comprising either the full variant sequence corresponding to positions 1 to 705 of SEQ ID NO:1 or position 51 to 705 of SEQ ID NO:1 or 75 to 705 of SEQ ID NO:1 with proper sequence alterations corresponding to actual amino acid alterations in the variant or any probe comprising a variant subsequence thereof having a length of at least about 100 base pairs under at least medium stringency conditions, but preferably at high stringency conditions. The variant polynucleotides of the present invention may also comprise silent mutations in addition to the mutations giving rise to the amino acid alterations described in the variant section above. Silent mutations are mutations in the three-letter code which do not give rise to a change in the amino acid, e.g. GTT to GAT, which both code for valine.

The polynucleotides encoding the lysozyme variants of the present invention include DNA and RNA. Methods for isolating DNA and RNA are well known in the art. DNA and RNA encoding genes of interest can be cloned in Gene Banks or DNA libraries by means of methods known in the art. Polynucleotides encoding polypeptides having antimicrobial and/or lysozyme activity of the invention are then identified and isolated by, for example, hybridization or PCR.

### Expression Vectors

The present invention also relates to expression vectors, in particular recombinant expression vectors, comprising a nucleic acid construct of the invention. Nucleic acid constructs of the invention comprise an isolated polynucleotide encoding the lysozyme or a variant lysozyme of the present invention, preferably operably linked to one or more control sequences which direct the expression of the coding sequence in a suitable host cell under conditions compatible with the control sequences. In creating the expression vector, the coding sequence is located in the vector so that the coding sequence is operably linked with the appropriate control sequences for expression. The control sequences may either be provided by the vector or by the nucleic acid construct inserted into the vector.

The control sequence may be an appropriate promoter sequence, a nucleotide sequence which is recognized by a host cell for expression of a polynucleotide encoding a polypeptide of the present invention. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell. Such promoters are well known in the art. The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention; such terminators are well known in the art. The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention, such leader sequences are well known in the art. The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention. The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention. It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound.

An isolated polynucleotide encoding the lysozyme or a variant lysozyme of the present invention may be manipulated in a variety of ways to provide for expression of the polypeptide. Manipulation of the polynucleotide sequence prior to insertion into a vector may be desirable or necessary depending on the expression vector. The techniques for modifying polynucleotide sequences utilizing recombinant DNA methods are well known in the art. Furthermore, tags which may aid purification or immobilization of the polypeptide may be added to the polypeptide. Such a tag may for example be a polyhistidine tag (His tag). Preferably, the tag located in the N-terminal or C-terminal of the polypeptide, and may be encoded by the vector. Alternatively, the tag may be located internally in the polypeptide, as long as it does not affect the functionality of the polypeptide.

The recombinant expression vector may be any vector (e.g. a plasmid, phagemid, phage or virus) that can be conveniently subjected to recombinant DNA procedures and can bring about the expression of the nucleotide sequence. The choice of the vector will typically depend on the compatibility of the vector with the host cell into which the vector is to be introduced.

The vectors may be linear or closed circular plasmids. The vector may be an autonomously replicating vector, i.e. a vector that exists as an extrachromosomal entity, the replication of which is independent of chromosomal replication, e.g. a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers that permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers that confer antibiotic resistance such as ampicillin, kanamycin, chloramphenicol or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention may contain an element(s) that permits stable integration of the vector into the host cell's genome or autonomous replication of the vector in the cell independent of the genome.

More than one copy of a nucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see for example Sambrook et al. (1989) supra).

In one embodiment of the present invention the plasmid vector may contain the following elements:
i) a signal peptide coding region (e.g. obtained from the genes for Bacillus NCIB 11837 maltogenic amylase, Bacillus stearothermophilus alpha-amylase, Bacillus licheniformis subtilisin, Bacillus licheniformis alpha-amylase, Bacillus stearothermophilus neutral proteases (nprT, nprS, nprM), and Bacillus subtilis prsA), followed by a polynucleotide sequence encoding the mature lysozyme variant. This sequence may be preceded by and operably linked to:
ii) a DNA sequence comprising a mRNA stabilising segment (e.g. derived from the Cryllla gene, as shown in WO 99/043835);
iii) a marker gene (e.g. a chloramphenicol resistance gene); and
iv) genomic DNA from Bacillus subtilis as 5′ and 3′ flanking segments upstream and downstream of the polynucleotide, respectively, to enable genomic integration by homologous recombination between the flanking segments and the Bacillus genome.

The vectors described above may also be useful in the generation and screening of the variants using the previously described mutagenesis procedures.

### Host Cells

The present invention also relates to a recombinant host cell comprising a polynucleotide encoding the lysozyme or a variant lysozyme of the invention, which is advantageously used in the recombinant production of the polypeptides. A vector comprising a polynucleotide sequence of the present invention is introduced into a host cell so that the vector is maintained as a chromosomal integrant or as a self-replicating extra-chromosomal vector as described earlier.

The host cell may be a prokaryote such as bacterial cells, an archaea or a eukaryote such as fungal cells, plant cells, insect cells, or mammalian cells.

Useful prokaryotes are bacterial cells such as gram positive bacteria including, but not limited to, a Bacillus cell, e.g. Bacillus alkalophilus, Bacillus amyloliquefaciens, Bacillus brevis, Bacillus circulans, Bacillus clausii, Bacillus coagulans, Bacillus halodurans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

The introduction of a vector into a bacterial host cell may, for instance, be effected by protoplast transformation (see for example Chang and Cohen (1979) Molecular General Genetics 168:111-115), using competent cells (see for example Young and Spizizin (1961) Journal of Bacteriology 81:823-829; or Dubnau and Davidoff-Abelson (1971) Journal of Molecular Biology 56:209-221), electroporation (see for example Shigekawa and Dower (1988) Biotechniques 6:742-751), or conjugation (see for example Koehler and Thorne (1987) Journal of Bacteriology 169:5771-5278).

In a preferred embodiment, the host cell is a fungal cell. “Fungi” as used herein includes the phyla Ascomycota, Basidiomycota, Chytridiomycota, and Zygomycota (as defined by Hawksworth et al. (1995) Ainsworth and Bisby's Dictionary of The Fungi (8th edition), CAB International, University Press, Cambridge, UK) as well as the Oomycota (as cited in Hawksworth et al. (1995) Ainsworth and Bisby's Dictionary of The Fungi (8th edition), CAB International, University Press, Cambridge, UK, page 171) and all mitosporic fungi (Hawksworth et al. (1995) Ainsworth and Bisby's Dictionary of The Fungi (8th edition), CAB International, University Press, Cambridge, UK). In a more preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport (eds) (1980) Soc. App. Bacteriol. Symposium Series No. 9).

In an even more preferred embodiment, the yeast host cell is a Candida, Hansenula, Kluyveromyces, Pichia, Saccharomyces, Schizosaccharomyces, or Yarrowia cell. In a most preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred embodiment, the yeast host cell is a Kluyveromyces lactis cell. In another most preferred embodiment, the yeast host cell is a Yarrowia lipolytica cell.

In another more preferred embodiment, the fungal host cell is a filamentous fungal cell. Filamentous fungi include all filamentous forms of the subdivision Eumycota and Oomycota (as defined by Hawksworth et al. (1995) Ainsworth and Bisby's Dictionary of The Fungi (8th edition), CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In an even more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, or Trichoderma. In a most preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.) cell. In another most preferred embodiment, the filamentous fungal host cell is a Humicola insolens, Humicola lanuginosa, Mucor miehei, Myceliophthora thermophila, Neurospora crassa, Penicillium purpurogenum, Thielavia terrestris, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238023 and Yelton et al. (1984) Proceedings of the National Academy of Sciences USA 81:1470-1474. Suitable methods for transforming Fusarium species are described by Malardier et al. (1989) Gene 78:147-156 and WO 96/00787. Yeast may be transformed using the procedures described by Becker and Guarente, Abelson and Simon (editors) Guide to Yeast Genetics and Molecular Biology, Methods in Enzymology 194:182-187, Academic Press, Inc., New York; Ito et al. (1983) Journal of Bacteriology 153:163; and Hinnen et al. (1978) Proceedings of the National Academy of Sciences USA 75:1920.

A particular embodiment of the present invention is a recombinant host cell transformed with a polynucleotide encoding a variant lysozyme of the present invention. Preferably, such a host cell does not contain an inherent lysozyme encoding gene, or such a gene has been disrupted. Thereby the recombinant variant lysozyme is the only lysozyme produced by the recombinant host cell of the present invention.

### Methods of Production

The present invention also relates to methods of producing a lysozyme variant, comprising: (a) cultivating a host cell of the present invention under conditions suitable for the expression of the variant; and (b) recovering the variant from the cultivation medium.

One embodiment of the present invention is a method for producing the lysozyme of SEQ ID NO:2, SEQ ID NO:4, SEQ ID NO:5, or SEQ ID NO:6 by recombinant means as indicated above.

In the production methods of the present invention, the host cells are cultivated in a nutrient medium suitable for production of the lysozyme variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g. in catalogues of the American Type Culture Collection). If the polypeptide is secreted into the nutrient medium, the polypeptide can be recovered directly from the medium. If the polypeptide is not secreted, it can be recovered from cell lysates.

One embodiment of the present invention is a method of producing a lysozyme or a variant of a parent lysozyme, wherein said lysozyme or variant has antimicrobial and/or lysozyme activity, said method comprising: a) culturing a cell under conditions suitable for expression of the lysozyme or variant, where said cell contains a polynucleotide sequence encoding a variant of a parent lysozyme in which said variant is altered in one or more (several) amino acid position(s) selected from the group consisting of positions: 3, 9, 10, 11, 17, 20, 25, 29, 32, 34, 37, 41, 47, 50, 60, 61, 67, 69, 73, 74, 81, 87, 88, 89, 90, 91, 92, 96, 97, 98, 101, 104, 108, 109, 111, and/or 124, and said polynucleotide sequence is prepared by mutagenesis of a parent polynucleotide sequence of SEQ ID NO:10R SEQ ID NO:3, or a parent polynucleotide sequence having at least 80% identity, such as, least 85%, at least 90%, at least 95%, at least 96%, at least 97%, at least 98%, at least 99% identity to the nucleotide sequence of SEQ ID NO:10R SEQ ID NO:3; and b) recovering the lysozyme variant from the cultivation medium. An optimized parent nucleic acid sequence may also be used, such as, provided in SEQ ID NO:3.

In an alternative aspect, the lysozyme or lysozyme variant is not recovered, but rather a host cell of the present invention expressing a variant is used as a source of the variant.

The lysozyme or lysozyme variant may be detected using methods known in the art that are specific for the expressed polypeptides. These detection methods may include use of specific antibodies, formation of an enzyme product, or disappearance of an enzyme substrate. For example, an enzyme assay may be used to determine the activity of the lysozyme or variant lysozyme as described herein in the Examples.

The resulting lysozyme or lysozyme variant may be recovered by methods known in the art. For example, the polypeptide may be recovered from the nutrient medium by conventional procedures including, but not limited to, collection, centrifugation, filtration, extraction, spray-drying, evaporation, or precipitation.

The lysozyme or the lysozyme variant of the present invention may be purified by a variety of procedures known in the art including, but not limited to, chromatography (e.g. ion exchange, affinity, hydrophobic, chromatofocusing, and size exclusion), electrophoretic procedures (e.g. preparative isoelectric focusing), differential solubility (e.g. ammonium sulfate precipitation), SDS-PAGE, or extraction (see e.g. J.-C. Janson and Lars Ryden (editors) (1989) Protein Purification VCH Publishers, New York) to obtain substantially pure lysozyme variants.

### Compositions

The present invention also relates to compositions comprising a variant lysozyme or a polypeptide having antimicrobial and/or lysozyme activity of the present invention and a carrier and/or an excipient. Preferably, the compositions are enriched in such a variant or polypeptide. The term “enriched” indicates that the antimicrobial and/or lysozyme activity of the composition has been increased, e.g. with an enrichment factor of 1.1 or more. Preferably, the compositions are formulated to provide desirable characteristics such as low color, low odor and acceptable storage stability.

The composition may comprise a variant or polypeptide of the present invention as the major enzymatic component, e.g. a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase.

The polypeptide compositions may be prepared in accordance with methods known in the art and may be in the form of a liquid, paste, gel or a dry formulation. For instance, the polypeptide may be formulated in the form of a granulate or a microgranulate. The variant or polypeptide to be included in the composition may be stabilized in accordance with methods known in the art. In a preferred embodiment the variant lysozyme is formulated in a liquid composition.

A preferred embodiment of the present invention is a feed composition comprising a lysozyme variant of the present invention. In particular a variant with improved stability at acidic pH and/or increased protease stability is preferred.

### Detergent Compositions

The present invention also encompasses detergent compositions comprising a lysozyme variant of the present invention. In particular a variant with improved stability and/or activity at alkaline pH and/or a variant with improved activity at low or moderate temperature is preferred. The detergent composition may be adapted for specific uses such laundry, in particular household laundry, dish washing or hard surface cleaning.

The detergent composition typically comprises conventional detergent ingredients such as surfactants, builders, bleaches, enzymes and other ingredients.

In a preferred embodiment the detergent composition comprises a lysozyme and a protease.

The detergent composition can be in any form, such as a solid, liquid, paste, gel or any combination thereof. The composition may be in the form of a tablet, bar or pouch, including multi-compartment pouches. The composition can be in the form of a powder, for example a free-flowing powder, such as an agglomerate, spray-dried powder, encapsulate, extrudate, needle, noodle, flake, or any combination thereof.

### Enzymes

In one aspect, the present invention provides a detergent additive comprising a lysozyme variant of the present invention. The detergent additive as well as the detergent composition may comprise one or more enzymes such as a protease, lipase, cutinase, amylase, carbohydrase, cellulase, pectinase, mannanase, arabinase, galactanase, xylanase, oxidase, e.g. a laccase, and/or peroxidase.

In general the properties of the selected enzyme(s) should be compatible with the selected detergent (i.e. pH-optimum, compatibility with other enzymatic and non-enzymatic ingredients, etc.), and the enzyme(s) should be present in effective amounts.

### Proteases:

Suitable proteases include those of animal, vegetable or microbial origin. Microbial origin is preferred. Chemically modified or protein engineered mutants are included. The protease may for example be a metalloprotease (EC 3.4.17 or EC 3.4.24) or a serine protease (EC 3.4.21), preferably an alkaline microbial protease or a trypsin-like protease. Examples of alkaline proteases are subtilisins (EC 3.4.21.62), especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include Alcalase®, Savinase®, Primase®, Duralase®, Esperase®, and Kannase® (Novozymes A/S), Maxatase®, Maxacal®, Maxapem®, Properase®, Purafect®, Purafect OxP®, FN2™, and FN3™ (Genencor International Inc.).

Protease enzymes may be incorporated into detergent compositions in accordance with the invention at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition

### Lipases:

Suitable lipases include those of bacterial or fungal origin. Chemically or genetically modified mutants of such lipases are included in this connection. The lipase may for example be triacylglycerol lipase (EC3.1.1.3), phospholipase A2 (EC 3.1.1.4), Lysophospholipase (EC 3.1.1.5), Monoglyceride lipase (EC 3.1.1.23), galactolipase (EC 3.1.1.26), phospholipase A1 (EC 3.1.1.32), Lipoprotein lipase (EC 3.1.1.34). Examples of useful lipases include a Humicola lanuginosa lipase, e.g. as described in EP 258 068 and EP 305 216; a Rhizomucor miehei lipase, e.g. as described in EP 238 023 or from H. insolens as described in WO 96/13580; a Candida lipase, such as a C. antarctica lipase, e.g. the C. antarctica lipase A or B described in EP 214 761; a Pseudomonas lipase, such as one of those described in EP 721 981 (e.g. a lipase obtainable from a Pseudomonas sp. SD705 strain having deposit accession number FERM BP-4772), in PCT/JP96/00426, in PCT/JP96/00454 (e.g. a P. solanacearum lipase), in EP 571 982 or in WO 95/14783 (e.g. a P. mendocina lipase), a P. alcaligenes or P. pseudoalcaligenes lipase, e.g. as described in EP 218 272, a P. cepacia lipase, e.g. as described in EP 331 376, a P. stutzeri lipase, e.g. as disclosed in GB 1,372,034, or a P. fluorescens lipase; a Bacillus lipase, e.g. a B. subtilis lipase (Dartois et al. (1993) Biochemica et Biophysica Acta 1131:253-260), a B. stearothermophilus lipase (JP 64/744992) and a B. pumilus lipase (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202. A preferred lipase variant is that of Humicola lanuginosa DSM 4109 as described in WO 00/60063. Especially preferred are the variants disclosed in the Example in WO 00/60063 with improved first wash performance., i.e., T231R+N233R;G91A+D96W+E99K+G263Q+L264A+1265T+G266D+T267A+L269N+R209P+T231R+N233R; N33Q+D96S+T231R+N233R+Q249R; E99N+N101S+T231R+N233R+Q249R; E99N+N101S+T231R+N233R+Q249R.

Suitable commercially available lipases include Lipex®, Lipolase® and Lipolase Ultra®, Lipolex®, Lipoclean® (available from Novozymes A/S), M1 Lipase™ and Lipomax™ (available from Genencor Inc.) and Lipase P “Amano” (available from Amano Pharmaceutical Co. Ltd.). Commercially available cutinases include Lumafast™ from Genencor Inc.

The lipases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Cutinases:

Potentially useful types of cutinases include (EC 3.1.1.74), e.g. a cutinase derived from Pseudomonas mendocina as described in WO 88/09367, or a cutinase derived from Fusarium solani pisi (described e.g. in WO 90/09446). Due to the lipolytic activity of cutinases they may be effective against the same stains as lipases. Commercially available cutinases include Lumafast™ from Genencor Inc.

The cutinases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Carbohydrases:

Carbohydrases covers glycoside hydrolases (EC 3.2.1.-) and polysaccharide lyases (EC 4.2.2.-). Glycoside hydrolases catalyze the hydrolysis of the glycosidic bond between two or more carbohydrates or between a carbohydrate and a non-carbohydrate moiety. Polysaccharide lyases catalyze the cleavage of polysaccharide chains by a beta elimination mechanism resulting in a double bond of the newly formed reducing end. Carbohydrases include for example amylases, hemicellulases, pectinases and cellulases described in more detail below. Other carbohydrases may be xanthanases or pullulanases.

Suitable xanthanases include those of bacterial or fungal origin. Chemically or genetically modified mutants are included. Sources of xanthanases are for example described in Cadmus et al. (1988) J of Industrial Microbiology and Biotechnology 4:127-133; EP0030393; and Hashimoto et al. (1998) Appl Environ Microbiol. 64:3765-3768.

Suitable pullulanases include those of bacterial or fungal origin. Chemically or genetically modified mutants are included. Sources of pullulanase are for example Dextrozyme® and Promozyme® D2 (Novozymes A/S).

### Amylases:

Amylases comprise e.g. alpha-amylases (EC 3.2.1.1), beta-amylases (EC 3.2.1.2) and/or glucoamylases (EC 3.2.1.3) of bacterial or fungal origin. Chemically or genetically modified mutants of such amylases are included in this connection. Alpha-amylases are preferred in relation to the present invention. Relevant alpha-amylases include, for example, α-amylases obtainable from Bacillus species, in particular a special strain of B. licheniformis, described in more detail in GB 1296839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Further examples of useful amylases are the alpha-amylases derived from Bacillus sp. strains NCIB 12289, NCIB 12512, NCIB 12513 and DSM 9375; the alpha-amylases shown in SEQ ID NO 1 and 2 of WO 95/26397 (hereby incorporated by reference); the AA560 alpha-amylase derived from Bacillus sp. DSM 12649 disclosed as SEQ ID NO:2 in WO 00/60060 (hereby incorporated by reference); and the variants of the AA560 alpha-amylase, including the AA560 variant disclosed in Examples 7 and 8 (hereby incorporated by reference).

Relevant commercially available amylases include Natelase®, Stainzyme®, Duramyl®, Termamyl®, Termamyl™ Ultra, Fungamyl® and BAN® (all available from Novozymes A/S, Bagsvaerd, Denmark), and Rapidase® and Maxamyl® P (available from DSM, Holland) and Purastar®, Purastar OxAm and Powerase™ (available from Danisco A/S).

Other useful amylases are CGTases (cyclodextrin glucanotransferases, EC 2.4.1.19), e.g. those obtainable from species of Bacillus, Thermoanaerobactor or Thermoanaerobacterium.

The amylases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Hemicellulases:

Suitable hemicellulases include enzymes with xylanolytiactivity, arabinolytic activity, galactolytic activity and/or mannolytic activity. The hemi-cellulases of the present invention may for example be selected from xylanases (EC 3.2.1.8, EC 3.2.1.32, and EC 3.2.1.136), xyloglucanases (EC 3.2.1.4 and EC 3.2.1.151), arabinofuranosidases (EC 3.2.1.55), acetylxylan esterases (EC EC 3.1.1.72), glucuronidases (EC 3.2.1.31, EC 3.2.1.56, 3.2.1.128 and 3.2.1.139), glucanohydrolase (EC 3.2.1.11, EC 3.2.1.83 and EC 3.2.1.73), ferulic acid esterases (EC 3.1.1.73), coumaric acid esterases (EC 3.1.1.73), mannanases (EC 3.2.1.25; EC 3.2.1.78 and EC 3.2.1.101), arabinosidase (EC 3.2.1.88), arabinanases (EC 3.2.1.99), galactanases (EC 3.2.1.89, EC 3.2.1.23 and 3.2.1.164) and lichenases (EC 3.2.1.73). This is, however, not to be considered as an exhausting list.

Mannananase is a preferred hemicellulase in relation to the present invention. Suitable mannanases include those of bacterial or fungal origin. Chemically or genetically modified mutants are included. In a preferred embodiment the mannanase is derived from a strain of the genus Bacillus, especially Bacillus sp. 1633 disclosed in positions 31-330 of SEQ ID NO:2 or in SEQ ID NO:5 of WO 99/64619 (hereby incorporated by reference) or Bacillus agaradhaerens, for example from the type strain DSM 8721. A suitable commercially available mannanase is Mannaway® produced by Novozymes A/S or Purabrite™ produced by Genencor a Danisco division.

Xylanase is a preferred hemicellulase in relation to the present invention. A suitable commercially available xylanase is Pulpzyme® HC (available from Novozymes A/S).

The hemicellulases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Pectinases:

Suitable pectinolytic enzymes include those described in WO 99/27083, WO 99/27084, WO 00/55309 and WO 02/092741.

Suitable pectate lyases include those of bacterial or fungal origin. Chemically or genetically modified mutants are included. In a preferred embodiment the pectate lyase is derived from a strain of the genus Bacillus, especially a strain of Bacillus substilis, especially Bacillus subtilis DSM14218 disclosed in SEQ ID NO:2 or a variant thereof disclosed in Example 6 of WO 02/092741 (hereby incorporated by reference) or a variant disclosed in WO 03/095638 (hereby incorporated by reference). Alternatively the pectate lyase is derived from a strain of Bacillus licheniformis, especially the pectate lyases disclosed as SEQ ID NO:8 in WO 99/27083 (hereby incorporated by reference) or variants thereof as described in WO 02/06442.

Suitable commercially available pectate lyases are Pectaway® or Pectawash® produced by Novozymes A/S.

The pectinolytic enzymes are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Cellulases:

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed “endoglucanases” (EC 3.2.1.4). Some xyloglucanases may also have endoglucanases activity and are also considered as suitable cellulases in the present invention. Suitable cellulases are disclosed in U.S. Pat. No. 4,435,307, which discloses fungal cellulases produced from Humicola insolens. Especially suitable cellulases are the cellulases having textile care benefits. Examples of such cellulases are cellulases described in European patent application No. 0 495 257.

Suitable mono-component endoglucanases may be obtained from one or more of the following species Exidia glandulosa, Crinipellis scabella, Fomes fomentarius, Spongipellis sp., Rhizophlyctis rosea, Rhizomucor pusillus, Phycomyces nitens, and Chaetostylum fresenii, Diplodia gossypina, Microsphaeropsis sp., Ulospora bilgramii, Aureobasidium sp., Macrophomina phaseolina, Ascobolus stictoides, Saccobolus dilutellus, Peziza, Penicillium verruculosum, Penicillium chrysogenum, and Thermomyces verrucosus, Trichoderma reesei aka Hypocrea jecorina, Diaporthe porthe syngenesia, Colletotrichum lagenarium, Xylaria hypoxylon, Nigrospora sp., Nodulisporum sp., and Poronia punctata, Cylindrocarpon sp., Nectria pinea, Volutella colletotrichoides, Sordaria fimicola, Sordaria macrospora, Thielavia thermophila, Syspastospora boninensis, Cladorrhinum foecundissimum, Chaetomium murorum, Chaetomium virescens, Chaetomium brasiliensis, Chaetomium cunicolorum, Myceliophthora thermophila, Gliocladium catenulatum, Scytalidium thermophila, Acremonium sp Fusarium solani, Fusarium anguioides, Fusarium poae, Fusarium oxysporum ssp. lycopersici, Fusarium oxysporum ssp. passiflora, Humicola nigrescens, Humicola grisea, Fusarium oxysporum, Thielavia terrestris or Humicola insolens. One preferred endoglucanase is disclosed in WO 96/29397 as SEQ ID NO:9 (hereby incorporated by reference) or an enzyme with at least 70% identity thereto and variants thereof as disclosed in Example 1 of WO 98/12307. Another preferred endoglucanase is disclosed in WO 91/017243 (SEQ ID NO:2) or endoglucanases variants as disclosed in WO 94/007998.

Endoglucanases with an anti-redeposition effect may be obtained from fungal endoglucanases lacking a carbohydrate-binding module (CBM) from a number of bacterial sources. Some sources are Humicola insolens, Bacillus sp. deposited as DSM 12648, Bacillus sp. KSMS237 deposited as FERM P-16067, Panibacillus polymyxa, and Panibacillus pabuli. Specific anti-redeposition endoglucanase are disclosed in WO 91/17244 (FIG. 14) (hereby incorporated by reference), WO 04/053039 (SEQ ID NO:2) (hereby incorporated by reference), JP 2000210081 (position 1 to 824 of SEQ ID NO:1) (hereby incorporated by reference).

Xyloglucanases with an anti-redeposition effect may be obtained from a number of bacterial sources. Some sources are Bacillus licheniformis, Bacillus agaradhaerens (WO 99/02663), Panibacillus polymyxa, and Panibacillus pabuli (WO01/62903). Suitable variants of xyloglucasnes are also described in PCT/EP2009/056875. A commercially available xyloglucanase is Whitezyme® (Novozymes A/S).

Commercially available cellulases include Celluclast® produced from Trichoderma reesei, Celluzyme® produced from Humicola insolens. Commercially available endoglucanases are Carezyme®, Renozyme®, Endolase® and Celluclean® (Novozymes A/S), and KAC-500(B)™ (Kao Corporation) and Clazinase™, Puradax EG L and Puradax HA (Danisco A/S).

Cellulases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Peroxidases/Oxidases:

Suitable peroxidases/oxidases include those of plant, bacterial or fungal origin. Chemically modified or protein engineered mutants are included. Examples of useful oxidases are laccases (EC 1.10.3.2). Examples of useful peroxidases include catalases (EC 1.11.1.6) and peroxidases from Coprinus, e.g. from C. cinereus, and variants thereof as those described in WO 93/24618, WO 95/10602, and WO 98/15257.

Commercially available peroxidases include Guardzyme™ (Novozymes A/S).

### Arylesterases:

Suitable arylesterases include those of plant, bacterial or fungal origin. Chemically modified or protein engineered mutants are included. Examples of useful arylesterase are for example obtained from M. Smegmatis as described in WO 05/056782.

### Surfactants

Typically, the detergent composition comprises (by weight of the composition) one or more surfactants in the range of 0% to 50%, preferably from 2% to 40%, more preferably from 5% to 35%, more preferably from 7% to 30%, most preferably from 10% to 25%, even most preferably from 15% to 20%. Preferred surfactants are anionic surfactants, non-ionic surfactants, cationic surfactants, zwitterionic surfactants, amphoteric surfactants, and mixtures thereof. Preferably, the major part of the surfactant is anionic. Suitable anionic surfactants are well known in the art and may comprise fatty acid carboxylates (soap), branced-chain, linear-chain and random chain alkyl sulfates or fatty alcohol sulfates or primary alcohol sulfates or alkyl benzenesulfonates such as LAS and LAB or phenylalknesulfonates or alkenyl sulfonates or alkenyl benzenesulfonates or alkyl ethoxysulfates or fatty alcohol ether sulfates or alpha-olefin sulfonate or dodecenyl/tetradecnylsuccinic acid. The anionic surfactants may be alkoxylated. The detergent composition may also comprise from 1 wt % to 10 wt % of non-ionic surfactant, preferably from 2 wt % to 8 wt %, more preferably from 3 wt % to 7 wt %, even more preferably less than 5 wt % of non-ionic surfactant. Suitable non-ionic surfactants are well known in the art and may comprise alcohol ethoxylates, and/or alkyl ethoxylaes, and/or alkylphenol ethoxylates, and/or glucamides such as fatty acid N-glucosyl N-methyl amides, and/or alkyl polyglucosides and/or mono- or diethanolamides or fatty acid amides. The detergent composition may also comprise from 0 wt % to 10 wt % of cationic surfactant, preferably from 0.1 wt % to 8 wt %, more preferably from 0.5 wt % to 7 wt %, even more preferably less than 5 wt % of cationic surfactant. Suitable cationic surfactants are well known in the art and may comprise alkyl quaternary ammonium compounds, and/or alkyl pyridinium compounds and/or alkyl quaternary phosphonium compounds and/or alkyl ternary sulphonium compounds. The composition preferably comprises surfactant in an amount to provide from 100 ppm to 5,000 ppm surfactant in the wash liquor during the laundering process. The composition upon contact with water typically forms a wash liquor comprising from 0.5 g/l to 10 g/l detergent composition. Many suitable surface active compounds are available and fully described in the literature, for example, in “Surface-Active Agents and Detergents”, Volumes 1 and 11, by Schwartz, Perry and Berch.

### Builders

The main role of a builder is to sequester divalent metal ions (such as calcium and magnesium ions) from the wash solution that would otherwise interact negatively with the surfactant system. Builders are also effective at removing metal ions and inorganic soils from the fabric surface, leading to improved removal of particulate and beverage stains. Builders are also a source of alkalinity and buffer the pH of the wash water to a level of 9.5 to 11. The buffering capacity is also termed “reserve alkalinity”, and should preferably be greater than 4.

The detergent compositions of the present invention may comprise one or more detergent builders or builder systems. Many suitable builder systems are described in the literature, for example in Powdered Detergents, Surfactant science series volume 71, Marcel Dekker, Inc. Builder may comprise from 0% to 60%, preferably from 5% to 45%, more preferably from 10% to 40%, most preferably from 15% to 35%, even more preferably from 20% to 30% builder by weight of the subject composition. Builders include, but are not limited to, the alkali metal, ammonium and alkanolammonium salts of polyphosphates (e.g. tripolyphosphate STPP), alkali metal silicates, alkaline earth and alkali metal carbonates, aluminosilicate builders (e.g. zeolite) and polycarboxylate compounds, ether hydroxypolycarboxylates, copolymers of maleic anhydride with ethylene or vinyl methyl ether, 1, 3, 5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Ethanole amines (MEA, DEA, and TEA may also contribute to the buffering capacity in liquid detergents.

### Bleaches

The detergent compositions of the present invention may comprise one or more bleaching agents. In particular powdered detergents may comprise one or more bleaching agents. Suitable bleaching agents include other photobleaches, pre-formed peracids, sources of hydrogen peroxide, bleach activators, hydrogen peroxide, bleach catalysts and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:

(1) other photobleaches, for example Vitamin K3;
(2) preformed peracids: suitable preformed peracids include, but are not limited to, compounds selected from the group consisting of percarboxylic acids and salts, percarbonic acids and salts, perimidic acids and salts, peroxymonosulfuric acids and salts, for example, Oxone, and mixtures thereof. Suitable percarboxylic acids include hydrophobic and hydrophilic peracids having the formula R—(C═O)O—O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;
(3) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include inorganic salts such as alkali metal silicate, carbonate or borate salts or mixtures thereof, or organic materials such as water-soluble or dispersible polymers, waxes, oils or fatty soaps. Useful bleaching compositions are described in U.S. Pat. Nos. 5,576,282, and 6,306,812;
(4) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof; and
(5) bleach catalysts that are capable of accepting an oxygen atom from peroxyacid and transferring the oxygen atom to an oxidizable substrate are described in WO2008/007319 (hereby incorporated by reference). Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof. The bleach catalyst will typically be comprised in the detergent composition at a level of from 0.0005% to 0.2%, from 0.001% to 0.1%, or even from 0.005% to 0.05% by weight.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

### Adjunct Materials

### Dispersants:

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents:

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent:

The detergent compositions of the present invention will preferably also contain additional components that may tint articles being cleaned, such as fluorescent whitening agent or optical brighterners. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of:
4,4′-bis-(2-diethanolamino-4-anilino-s-triazin-6-ylamino) stilbene-2,2′-disulphonate,
4,4′-bis-(2,4-dianilino-s-triazin-6-ylamino) stilbene-2.2′-disulphonate,
4,4′-bis-(2-anilino-4(N-methyl-N-2-hydroxy-ethylamino)-s-triazin-6-ylamino) stilbene-2,2′-disulphonate,
4,4′-bis-(4-phenyl-2,1,3-triazol-2-yl)stilbene-2,2′-disulphonate,
4,4′-bis-(2-anilino-4(1-methyl-2-hydroxy-ethylamino)-s-triazin-6-ylamino) stilbene-2,2′-disulphonate and,
2-(stilbyl-4″-naptho-1.,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl)disulphonate.

Also preferred are fluorescent whitening agents is the commercially available Parawhite KX, supplied by Paramount Minerals and Chemicals, Mumbai, India.

Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Fabric Hueing Agents:

The detergent compositions of the present invention may also include fabric hueing agents such as dyes or pigments which when formulated in detergent compositions can deposit onto a fabric when said fabric is contacted with a wash liquor comprising said detergent compositions, thus altering the tint of said fabric through absorption of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 05/03274, WO 05/03275, WO 05/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch.

### Soil Release Polymers:

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 09/087,523 (hereby incorporated by reference). Furthermore random graft co-polymers are suitable soil release polymers Suitable graft co-polymers are described in more detail in WO 07/138,054, WO 06/108856 and WO 06/113314 (hereby incorporated by reference). Other soil release polymers are substituted polysaccharide structures especially substituted cellulosic structures such as modified cellulose deriviatives such as those described in EP 1867808 or WO 03/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxylpropyl methyl cellulose, ester carboxy methyl cellulose, and mixtures thereof.

### Anti-Redeposition Agents:

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of:

(i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (iii) both (i) and (ii). All the ingredients are described in WO 07/130,562 hereby incorporated by reference in its entirety further polymers useful in detergent formulations are described in WO 07/149,806, which is hereby incorporated by reference in its entirety.

In another aspect the detergent is a compact granular (powdered) detergent comprising a) at least about 10%, preferably from 15 to 60% by weight of the composition, of surfactant selected from anionic surfactants, non ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeiolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

### Uses

The Opisthocomus hoazin lysozyme or lysozyme variant, or a composition thereof, of the present invention may be used in several applications to degrade a material comprising a peptidoglycan or a chitodextrin by treating the material with the lysozyme or composition thereof (see for example Proctor and Cunningham, (1988) Critical Reviews in Food Science and Nutrition 26:359-395; Carini et al. (1985) Microbiol. Alimen. Nutr. 3:299-320; Hughey and Johnson (1987) Appl. Environ. Microbiol. 53:2165-2170; Cunningham et al. (1991) World's Poultry Science Journal 47:141-163).

Examples of preferred uses of the lysozyme or compositions thereof of the present invention are given below. The dosage of the lysozyme and other conditions under which the lysozyme is used may be determined on the basis of methods known in the art.

A lysozyme of the present invention may be used as antimicrobial agents. One aspect of the present invention is a method for reducing microbial contamination, comprising treating a microbial contaminated surface with a lysozyme of the present invention.

To assess whether a lysozyme of the present invention is capable of acting as an antimicrobial agent it can be tested in a turbidity assay. In this assay it is tested whether the lysozyme is capable of degrading microbial cells e.g. a dried substrate of Exiguobacterium undae cells (isolated from a smelly sock) or Clostridium perfringens cells dissolved in buffer or detergent, and thereby reducing the optical density (OD) at for example 540 nm, when compared to a microbial suspension only treated with buffer.

A lysozyme of the present invention is preferably incorporated into and/or used together with detergent compositions as described above.

When washing is performed repeatedly at temperatures below 60° C. there is an increased risk of malodour in the washing machine (laundry as well as dishwashing) and on the textiles or items washed in the machine. This malodour is likely to be caused by microbial organisms such as bacteria, fungi, algae or other unicellular organisms growing in the washing machine. The present invention provides a method of reducing microbial contamination on a surface, such as a textile garment or hard surface such as metal, plastic or rubber parts in a washing machine or dish washing machine, bathroom tiles, floors, table tops, drains, sinks and washbasin, by treating the microbially contaminated surface with a lysozyme or lysozyme composition of the present invention. Such a treatment is also expected to reduce the malodour on textiles and hard surfaces containing microbial contamination.

The reduction of microbial contamination can be assessed in several ways, for example by letting a panel assess whether the smell has been decreased, alternatively a sample may be taken from the surface and cultivated to assess whether the microbial count has been reduced as a result of the treatment compared to a treatment without lysozyme.

Furthermore, the invention relates to a process for laundering of fabrics comprising treating fabrics with a washing solution containing a detergent composition and a lysozyme or a lysozyme composition of the invention. The laundering treatment can for example be carried out in a machine washing process or in a manual washing process. The washing solution can for example be an aqueous washing solution containing the detergent composition and with a pH between 3 and 12, preferably between pH 7 and 12, more preferably between pH 8 and 10.

The fabrics subjected to the methods of the present invention may be conventional washable laundry, for example household laundry. Preferably, the major part of the laundry is garments and fabrics, including knits, wovens, denims, yarns, and towelling, made from cotton, cotton blends or natural or manmade cellulosics (e.g. originating from wood pulp) or blends thereof. Examples of blends are blends of cotton or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell).

A lysozyme of the invention may also be used in animal feed. In an embodiment, the present invention provides a method for preparing an animal feed composition comprising adding a lysozyme of the present invention to one or more animal feed ingredients. Preferably, the animal feed does not contain antibiotics.

A lysozyme of the present invention may for example be used to stabilize the healthy microflora of animals, in particular livestock such as, but not limited to, cattle, dear, goats, pigs, poultry, rabbits and sheep, but also in fish by suppressing growth/intestinal colonization of pathogens such as Clostridium perfringens, Escherichia coli and Salmonella, such as Salmonella typhimurium and Salmonella Mbandaka. In a preferred embodiment, the lysozyme replaces antibiotics in animal diets. In a preferred embodiment a lysozyme is applied to chicken and has anti-microbal activity against Clostridium perfringens. In a further embodiment a lysozyme of the present invention is used as a feed additive, where it may provide a positive effect on the microbial balance of the chicken digestive tract and in this way improve animal performance.

A lysozyme of the present invention may also be used in animal feed as feed enhancing enzymes that improve feed digestibility to increase the efficiency of its utilization according to WO 00/21381 and WO 04/026334.

A lysozyme of the present invention may be used for disinfection and for preventing or removing biofilm on a surface according to U.S. Pat. No. 6,777,223.

A lysozyme of the present invention may also be used to selectively inhibit the uncontrolled growth of Clostridium tyrobutyricum during the maturation of cheeses, in particular those made from pressed and cooked curds, e.g. Swiss Cheese, Parmesan, Edam, Gouda, Cheddar, and many others.

A lysozyme of the present invention may also be used in oral care. For example, lysozyme can be used alone or in combination with other enzymes or even antimicrobial peptides in toothpaste or other oral care products. The polypeptides may be introduced into the oral cavity or applied to an article that is to be introduced into the oral cavity. See for example WO 08/124,764.

A lysozyme of the present invention may also be used in topical treatment of dystrophic and inflammatory lesions of the skin and soft tissues. See for example Palmieri and Boraldi (1977) Arch. Sci. Med. (Torino) 134:481-485.

A lysozyme of the present invention may also be used in skin care. For example, the polypeptide is applied to the skin of a patient suffering from a skin infection, such as acne. The lysozyme may also be used in a wound dressing, which is applied to wounded skin, for example, to aid in healing of the wound. See, for example, U.S. Application No. 20080254079.

A lysozyme of the present invention may also be used in lipstick, lip balm, lip gel, or lip gloss. For example, such products can be used for treatment of a localized lip infection, for example, a cold sore. See, for example, U.S. Application No. 20080254079.

A lysozyme of the present invention may also be used in the treatment of bronchopulmonary diseases.

A lysozyme of the present invention may also be used to control microbial growth in a fermentation process, such as, in making ethanol or other products from biomass. See, for example, WO 2007/109750. Accordingly, the lysozyme may be used, e.g., in a process for producing a fermentation product comprising (a) liquefying and/or saccharifying a carbohydrate material and (b) fermenting using a fermentation organism, wherein a lysozyme of the present invention is applied to the fermentation process before, during and/or after fermentation concentrations sufficient to kill and/or inhibit growth of bacterial cells.

A lysozyme of the present invention may also be used in wine making, to control or inhibit microbial contamination.

A lysozyme of the present invention may also be used as digestive enzymes or digestive aids. A lysozyme of the present invention may also be used to improve the use of dead/live bacteria as a food source, e.g., by controlling undesirable microbial contaminants.

A lysozyme of the present invention may also be used as a therapeutic in a human or other animal, e.g., to control or inhibit bacterial overgrowth in the intestines of a human suffering from a disease, e.g., pancreatic disease or an immuno compromised patient.

A lysozyme of the present invention may also be used in controlling microbial growth in a fish or shrimp farm.

### Plants

The present invention also relates to plants, e.g., a transgenic plant, plant part, or plant cell, comprising an isolated polynucleotide encoding a polypeptide having lysozyme activity. The polypeptide may be recovered from the plant or plant part. Alternatively, the plant or plant part containing the recombinant polypeptide may be used for control or inhibiting microbial infestation in the plant or in an animal digesting the plant or plant material as a feed source.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part.

Likewise, plant parts such as specific tissues and cells isolated to facilitate the utilisation of the invention are also considered plant parts, e.g., embryos, endosperms, aleurone and seeds coats.

Also included within the scope of the present invention are the progeny of such plants, plant parts, and plant cells.

The transgenic plant or plant cell expressing a polypeptide of the present invention may be constructed in accordance with methods known in the art. In short, the plant or plant cell is constructed by incorporating one or more (several) expression constructs encoding a polypeptide of the present invention into the plant host genome or chloroplast genome and propagating the resulting modified plant or plant cell into a transgenic plant or plant cell.

The expression construct is conveniently a nucleic acid construct that comprises a polynucleotide encoding a polypeptide of the present invention operably linked with appropriate regulatory sequences required for expression of the nucleotide sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the polypeptide is desired to be expressed. For instance, the expression of the gene encoding a polypeptide of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, and the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588). Likewise, the promoter may inducible by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a polypeptide of the present invention in the plant. For instance, the promoter enhancer element may be an intron that is placed between the promoter and the nucleotide sequence encoding a polypeptide of the present invention. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated the expression construct are selected and regenerated into whole plants according to methods well-known in the art. Often the transformation procedure is designed for the selective elimination of selection genes either during regeneration or in the following generations by using, for example, co-transformation with two separate T-DNA constructs or site specific excision of the selection gene by a specific recombinase.

The present invention is further described by the following examples that should not be construed as limiting the scope of the invention.

### EXAMPLES

### Materials and Methods

### Turbidity Assay

The activity of lysozyme may be determined by measuring the decrease (drop) in absorbance/optical density of a solution of resuspended Micrococcus lysodeikticus ATTC No. 4698 (Sigma-Aldrich M3770) measured in a spectrophotometer at 540 nm.

Preparation of Micrococcus lysodeikticus Substrate

Before use the cells are resuspended in citric acid—phosphate buffer pH 4.4 to a concentration of 0.5 mg cells/mL and the optical density (OD) at 540 nm is measured. The cell suspension is then adjusted so the cell concentration equaled an OD540=1.0. The adjusted cell suspension is then stored cold before use. Resuspended cells should be used within 4 hours.

### Measurement of Lysozyme Antimicrobial Activity in the Turbidity Assay

The lysozyme sample to be measured is diluted to a concentration of 100-200 mg enzyme protein/L in citric acid—phosphate buffer pH 4.4, and kept on ice until use. In a 96 well microtiterplate (Nunc) 200 μL of the substrate is added to each well, and the plate is incubated at 37° C. for 5 minutes in a VERSAmax microplate reader (Molecular Devices). Following the incubation the absorbance of each well is measured at 540 nm (start value). To start the activity measurement 20 μL of the diluted lysozyme samples is added to the 200 μL substrate in each well and kinetic measurement of absorbance at 540 nm is initiated for minimum 30 minutes up to 24 hours at 37° C. The measured absorbance at 540 nm is monitored for each well and over time a drop in absorbance is seen if the lysozyme has antimicrobial activity. The larger the drop in absorbance is the larger was the lysozyme antimicrobial activity.

Preparation of citric acid—phosphate buffer pH 4.4

### Stock:

Mix 55.90 mL 0.1 M citric acid with 44.10 mL 0.2 M Na₂HPO₄

Adjust pH with HCl or NaOH to pH 4.4.

In order to compare results from the turbidity assay the samples to be compared should preferably be tested in the same experimental run using the same buffer and substrate batch.

### Variations in the Lysozyme Antimicrobial Activity in the Turbidity Assay

The assay described above, is the preferred standard microbial activity test for the present invention. However, for lysozyme variants which are generated to achieve an improved property, the assay may be adapted.

To test improved thermal activity the incubation temperature can be increased to the desired temperature, e.g. from 45° C. to 110° C. If the temperature exceeds 50° C., the sample must be incubated in a external heat-source instead of directly in the spectrophotometer measurements are then performed by measuring a start value and an end value.

To test improved activity at low or moderate temperatures the incubation temperature can be changed to the desired temperature, e.g. from 0° C. to 40° C.

To test improved temperature stability the lysozyme sample may be pre-incubated at a desired temperature, e.g. 45° C. to 110° C. for 30 min to 24 hours, and then cooled down to the temperature of the assay described above before measuring the activity.

To test improved pH activity the pH may be increased to for example pH 7.5 to 12 or decreased pH for example from 2 to 5.5.

To test improved pH stability the lysozyme sample may be pre-incubated at a desired pH for 30 min to 24 h, and then returned to the pH of the assay described above before measuring the activity.

To test increased resistance to protease degradation the lysozyme sample may be pre-incubated with pepsin at 0.5 to 2 mg/ml, preferably from 1 to 1.5 mg/ml or with a serine-proteases such as 10 mg/L Savinase® at 25-40° C. for 30 min to 24 h prior to conducting the assay described above.

The temperature and pH optimum of a lysozyme may also be assessed using the turbidity assay. For temperature optimum assessment the assay is run in a range of temperatures, for example from 5° C. to 80° C., while the pH is maintained at 6. For pH optimum the pH of the assay is varied over a range, for example pH 2 to 12 while the temperature is maintained at 37° C.

To test non-enzymatic glycation, a purified lysozyme is incubated with glucose under the following conditions: 0.5 mg/ml enzyme in 0.1M HEPES pH 7.5 is mixed with 1M glucose and incubated at 50° C. for 5 hr. The activity of the lysozyme is measured before and after incubation.

### Thermostability Screen

Purified samples of WT lysozyme and variants in the form of supernatants were diluted in Citric acid—phosphate buffer pH 4.4. Lysozyme thermostability was tested by incubation of purified lysozyme WT and variants thereof in a thermal Cycler (Applied Biosystems) for 76 min at 95° C. using. Simultaneously, control samples were incubated on ice. Immediately after incubation samples were put on ice. Residual activity was assessed as described for the turbidity assay using Micrococcus luteus as substrate except that activity was measured for 8 minutes.

### Example 1

### Expression of Opisthocomus Hoazin Lysozyme and Variants Thereof.

### Summary

The lysozyme from the folivorous bird Opisthocomus hoazin was expressed in Aspergillus oryzae. 31 variants covering modifications for altering specificity, pH optimum and stability were made. All variants were enzymatically active—including anti-glycation variants made with the aim of enhancing pelleting stability and variants aiming at modifying specificity to resemble Hen Egg White lysozyme. 6 variants were made that in comparison to WT showed improved thermostability.

### Heterologous Expression in Aspergillus

Using the published Genbank entry as starting point (AAA73935.1, Genbank entry, published in Kornegay et al., 1994, according to which genomic DNA was extracted from a frozen hoatzin tissue sample provided by the Louisiana State University Museum of Vertebrate Zoology collection (Opisthocomus hoazin (GenBank® accession number L36032) an artificial coding region (CDR) was designed adapted to Aspergillus oryzae codon usage.

The CDR was amplified by PCR and cloned into an expression vector using InFusion cloning. Resulting clones were sequenced on both strands and transformed into an Aspergillus oryzea expression host.

Transformants were grown for 4 days in YPM supplemented MTP and resulting supernatants analyzed by SDS-page.

The codon optimized CDR gave rise to expression of a protein with a Mw a little higher that the predicted Mw of 14.3 kDa

The transformant was fermented in shake flasks and purified. Edman degradation on the purified protein indicated an N-terminus corresponding to the expected and Mass spectroscopy data fitted the predicted molecular weight.

### Determination of Structure

The purified enzyme from above was used for X-ray structure determination. The structural coordinates are provided in FIG. 1.

### Example 2

### In Vitro Stability of Hoazin Lysozyme

Residual activity of hen egg white (Hew) and hoatzin lysozymes was used to determine the stability of the hoatzin lysozyme as compared to Hew lysozyme.

Both lysozymes were incubated 0, 15, 30 and 60 minutes in artificial gastric juice. The turbidity assay activity measurements from each incubation time point was compared to lysozyme standard curves and translated into a lysozyme concentration estimate. The results are indicated in Table 3 below.

TABLE 3

Gastric incubation time (min)

| | 0 | 15 | 30 | 60 |
|---|---|---|---|---|
| Hew lysozyme | 54 (100%) | 30 (56%) | 16 (30%) | 4.0 (7.5%) |
| concentration | | | | |
| Hoatzin lysozyme | 52 (100%) | 49 (94%) | 48 (92%) | 41 (78%) |
| concentration | | | | |

Number in parentheses describes the percentage decrease in lysozyme concentration

### Example 3

### Antimicrobial Activity of the Opisthocomus Hoazin Lysozyme

### Assay for Determination of Antimicrobial Activity:

### Inoculation and Preparation of Cultures:

Clostridium perfringens is inoculated into test tubes with 10 ml LB media. It is incubated in an anaerobic incubator over night at 37° C. without shaking.

The culture is transferred to Eppendorf tubes that are centrifugated at 12.000 RPM for 2 minutes.

The supernatant is discarded and pellets pooled. 0.9% saline is added to the Eppendorf tube with pellet and vortexed briefly.

Dilutions of 10×, 100×, 1000×, and 10000× are made and OD405 nm measured.

### Lysozyme Treatment:

10 μl lysozyme or buffer control is mixed with 100 μl bacterial suspension in a microtiter dish. This is incubated at 37° C. for 1 hour at 750 rpm in an Eppendorf thermomixer.

75 μl from each well is plated on TY plates including buffer controls as well as lysozyme control. This is incubated in an anaerobic incubator over night at 37° C.

The number of colony forming units (CFU) is counted on each plate.

Hoatzin lysozyme antibacterial activity against C. perfringens was tested at pH 4, 5 and 6.

Two different batches were tested using the above assay. The results are shown in Table 4

TABLE 4
Antibacterial effect of 100 ppm Hoatzin lysozyme batch A
against C. perfringens at pH 4, 5 and 6. pH

| | CFU Hoatzin | | |
|---|---|---|---|
| pH | lysozyme treated | CFU Untreated | % cells killed |
| 4 | 283 | 304 | 7 |
| 5 | 270 | 435 | 61 |
| 6 | 361 | 417 | 16 |

A reduction of live C. perfringens cells was observed after incubation with 100 ppm Hoatzin lysozyme Batch A.

TABLE 5
Antibacterial effect of Hoatzin lysozyme Batch B against
C. perfringens at pH 4, 5 and 6. Numbers are
averages from two days.

| | CFU Hoatzin | | |
|---|---|---|---|
| pH | lysozyme treated | CFU Untreated | % cells killed |
| 4 | 1561 | 1572 | 1 |
| 5 | 712 | 1248 | 43 |
| 6 | 1109 | 1412 | 21 |

A reduction of live C. perfringens cells were observed after incubation with 52 ppm Hoatzin lysozyme Batch B for two days.

### Example 4

### Expression of Opisthocomus Hoazin Lysozyme Variants

### KEX-B Site Insertion

Production of variants started by creating a variant aiming at introducing a KEX-B site between the signal and the mature region. The site was introduced at the C-terminal side of the E in position 1 (*1 aK *1 bR) thus generating an N-terminal sequence corresponding to EKR. This variant was used as template for the remaining variants.

### Variant Production

Variants were made using the KexB wild type (*1aK+*1bR) by well known methods.

The following variants were produced:

TABLE 6
Variants produced

| Designation | Modifications | Concept |
|---|---|---|
| KexB WT | *1aK + *1bR | |
| | Additional modifications | |
| 1 | G19N + E21T; | Addition of N-glycosylation |
| 2 | H34N; | Addition of N-glycosylation |
| 4 | K60R + | |
| | Y61WWYYGTFQINSRW | |
| | WCNDGKAFSRSTRGG | |
| 5 | K60R + Y61W | K60R addresses glycation |
| | | susceptibility; Y61W |
| | | addresses specificity. |
| 6 | K124R; | Glycation susceptibility |
| 7 | K124T; | Glycation susceptibility |
| | | Improved stability |
| 8 | K124S | Reduced glycation |
| | | Improved stability |
| 9 | Y108V | Substrate specificity |
| 10 | A101P | Improved stability |
| 11 | I97V | Substrate specificity |
| 12 | K96R | Glycation susceptibility |
| 13 | K92A | Glycation susceptibility |
| 14 | D90A | Broader pH activity profile |
| 15 | D89Q | Alkaline stability |
| 16 | E88T | Alkaline stability |
| 17 | G74A | Improved stability |
| 18 | K67R | Glycation susceptibility |
| 19 | Y61W | Substrate specificity |
| 20 | K60R | Glycation susceptibility |
| 21 | R50T | Broader pH activity profile |
| 22 | G47P | Improved stability |
| 23 | E41R | Alkaline stability |
| 24 | E41N | Alkaline stability |
| 25 | D37N | Alkaline stability |
| 26 | H34W | Improved stability |
| 27 | V32A | Improved stability |

Variants were transformed into an A. oryzae host, expression estimated by SDS-page and activity evaluated using a the turbidity assay with Micrococcus luteus as substrate., All generated variants were active.

### Example 5

### Glycosylation of Variants

The variants aiming at N-glycosylation all showed shifts to higher molecular weights in SDS-page thus suggesting that these variants were glycosylated.

TABLE 7
Glycosylated variants

| Mutation | Designation | Concept |
|---|---|---|
| G19N + E21T | 1 | Addition of N-glycosylation |

### Example 6

### Thermostability of Variants

### Thermostability Assays

Initially the thermostability of purified WT was assessed. Several attempts were needed to reach an inactivation percentage of around 80%. Approximately 20% residual activity was obtained after incubation at 95° C. for 76 min.

All variants were inoculated in a single FT X-14 shake flask, fermented for three days at 34° C. and supernatants harvested. Aliquots of the supernatants were used for relative stability assessment using WT supernatant as reference.

The following variants showed improved thermostability and candidate for purification, DSC and subsequent combination:

TABLE 8
Variants with improved thermostability

| Designation | Mutation | Result compared to real WT |
|---|---|---|
| 12 | K96R | + |
| 13 | K92A | + |
| 17 | G74A | +++ |
| 20 | K60R | ++ |
| 22 | G47P | ++ |
| 25 | D37N | + |

1-22. (canceled)

23. A variant lysozyme, wherein the variant lysozyme:
(a) comprises an amino acid modification at one or more positions selected from the group consisting of positions 3, 9, 10, 11, 17, 20, 25, 29, 32, 34, 37, 41, 47, 50, 60, 61, 67, 69, 73, 74, 81, 87, 88, 89, 90, 91, 92, 96, 97, 98, 101, 104, 108, 109, 111, and/or 124 (using SEQ ID NO:5 for numbering),
(b) comprises an amino acid sequence which is at least 80% identical to the polypeptide corresponding to the mature polypeptide of SEQ ID NO:2, SEQ ID NO:4 or SEQ ID NO:5 or to a polypeptide shown in SEQ ID NO:6, with the proviso that it is not the lysozyme of SEQ ID NO:2, SEQ ID NO:4 or SEQ ID NO:5, and
(c) has lysozyme and/or antimicrobial activity.

24. The variant lysozyme of claim 23, which comprises one or more of the following modifications: 3F, 9A, 10R, 11T, 17L, 20Y, 25L, 29L, 32A, 34W, 37N, 41N,R, 47P, 50T, 60R, 61W, 67R, 69P, 73N, 74A, 81A, 87I, 88T, 89Q, 90A, 91V, 92A, 96R. 97V, 98V, 101P, 104I, 108V, 109A, 111R, and/or 124S,T.

25. The variant lysozyme of claim 24, which comprises one or more of the following modifications: I3F, V9A, K10R, I11T, F17L, F20Y, I25L, I29L, V32A, H34W, D37N, E41N,R, G47P, R50T, K60R, Y61W, K67R, S69P, D73N, G74A, E81A, L87I, E88T, D89Q, D90A, I91V, K92A, K96R. I97V, A98V, A101P, L104I, Y108V, G109A, K111R, and/or K124S,T.

26. The variant lysozyme of claim 23, wherein the variant:
(a) comprises a modification at one or more of the following positions 3, 9, 11, 17, 20, 25, 29, 32, 34, 41, 47, 74, 81, 87, 91, 98, 104, 124 (using SEQ ID NO:5 for numbering), and
(b) the variant lysozyme has improved stability as compared to the parent lysozyme.

27. The variant lysozyme of claim 23, wherein the variant lysozyme:
(a) comprises a modification at one or more of the following positions 10, 60, 67, 92, 96, 111, 124, and
(b) the variant lysozyme has reduced glycation susceptibility as compared to the parent lysozyme.

28. The variant lysozyme of claim 23, wherein the variant lysozyme:
(a) comprises a modification at one or more of the following positions 37, 41, 69, 73, 81, 88, 89, 101 (using SEQ ID NO:5 for numbering), and
(b) the variant lysozyme has improved stability at alkaline pH as compared to parent lysozyme.

29. The variant lysozyme of claim 23, wherein the variant lysozyme:
(a) comprises a modification at one or more of the following positions 50 and 90 (using SEQ ID NO:5 for numbering), and
(b) the variant lysozyme has a broader pH activity profile as compared to the parent lysozyme.

30. The variant lysozyme of claim 23, wherein the variant lysozyme:
(a) comprises a modification at one or more of the following positions 61, 97, 108, 109 (using SEQ ID NO:5 for numbering), and
(b) the variant lysozyme has improved substrate specificity as compared to the parent lysozyme.

31. The variant lysozyme of claim 23, wherein the variant lysozyme comprises amino acids corresponding to D51 and E35.

32. The variant lysozyme of claim 23, wherein the variant lysozyme comprises modifications of residues in one or more of the binding sites:
D100 (Binding Site A);
D100 (Binding Site B);
N58, Y61, W62, P106 (Binding Site C);
Q56, Y108, D51 (Binding Site D);
N44, Q56, E35 (Binding Site E); and
H34, D37, H113 (Binding Site F);
wherein the amino acid sequence of the variant lysozyme of the present invention has one or more of the following amino acids in the binding sites:
(A and B): D100 (no change);
(C′): 61Y or 61W and no change in N58 and P106, or specifically Y61W;
(D′): 108Y or 108V and no change in Q56 and D51, or specifically Y108V;
(E′): no change in N44, Q56, and E35; and/or
(F′): 34H or 34W, 37D or 37N, and no change in H113, or specifically H34W, D37N, and H34W+D37N.

33. The variant lysozyme of claim 23, wherein the variant lysozyme comprises:
no changes in the amino acids corresponding to E35, D51 and D100;
no changes in E35 and D51 in combination with binding site C′;
no changes in E35 and D51 in combination with binding site D′;
no changes in E35 and D51 in combination with binding site E′;
no changes in E35 and D51 in combination with binding site F′;
no changes in E35 and D51 in combination with 34H or 34W, 37D or 37N, 61Y or 61W, 108Y or 108V, and
no change in N44, Q56, N58, W62, D100, P106, and H113 (using SEQ ID NO:5 for numbering).

34. The variant lysozyme of claim 23, wherein the variant lysozyme is a variant of a parent lysozyme, and wherein the parent lysozyme comprises an amino acid sequence comprising SEQ ID NO:2, SEQ ID NO:4, SEQ ID NO:6; or wherein the parent lysozyme comprises an amino acid sequence shown in SEQ ID NO:6 or wherein the parent lysozyme is the Opisthocomus hoazin lysozyme.

35. A detergent composition comprising the variant lysozyme of claim 23 and a surfactant.

36. A feed composition comprising the variant lysozyme of claim 23 and a feed component.

37. A method for reducing microbial contamination, comprising treating a microbially contaminated surface or composition with a variant lysozyme of claim 23.

38. An isolated polynucleotide sequence encoding the variant lysozyme of claim 23.

39. A recombinant host cell comprising an expression vector comprising the polynucleotide sequence of claim 38.

40. A method for producing a variant lysozyme having lysozyme activity, said method comprising:
(a) cultivating the host cell of claim 39 under conditions suitable for the expression of the variant lysozyme; and
(b) recovering the variant lysozyme from the cultivation medium.


SEQUENCE LISTING (6 sequences)

SEQ ID NO: 1 - DNA, 435 bp, Opisthocomus hoazin
  CDS (1)..(435)
  sig_peptide (1)..(57)
  mat_peptide (58)..(435)
atgctcttcttcggctttcttctggccttcctctcggctgtgccaggcactgagggagaaatcatcccccgatgtgagttggtgaagatcctgcgtgagcatggctttgagggcttcgagggcacaaccatagctgactggatctgcctggtgcaacacgagagcgattacaacaccgaagcgtataacaacaatggtccaagcagggactacgggatctttcagatcaacagcaagtactggtgtaatgatggcaagaccagtggagccgtcgacggctgccacatcagttgctcagagcttatgacaaatgatcttgaggacgatattaagtgtgccaagaagattgctagagatgctcacggcctcactccctggtacggctggaaaaaccattgcgagggcagagacctgagttcctacgtcaagggctgc

SEQ ID NO: 2 - protein, 145 aa, Opisthocomus hoazin
MLFFGFLLAFLSAVPGTEGEIIPRCELVKILREHGFEGFEGTTIADWICLVQHESDYNTEAYNNNGPSRDYGIFQINSKYWCNDGKTSGAVDGCHISCSELMTNDLEDDIKCAKKIARDAHGLTPWYGWKNHCEGRDLSSYVKGC

SEQ ID NO: 3 - DNA, 378 bp, Artificial
  Synthetic Construct
gagatcattcctcgctgtgagctcgtcaaaatcttgagggagcacggcttcgaaggcttcgaaggtaccactatcgcagactggatctgtttggtccagcacgaatcggactataacacagaggcctacaacaacaacggtccctccagggactacggcattttccagatcaactcgaagtattggtgtaacgatggaaagacttccggtgcagtcgacggttgtcacatttcgtgttccgagctcatgacgaacgatttggaggacgacatcaagtgtgccaagaaaatcgccagggacgcccacggattgacaccttggtacggctggaaaaaccattgtgaaggccgagatttgtcctcctatgtgaagggttgt

SEQ ID NO: 4 - protein, 126 aa, Artificial
  Synthetic Construct
EIIPRCELVKILREHGFEGFEGTTIADWICLVQHESDYNTEAYNNNGPSRDYGIFQINSKYWCNDGKTSGAVDGCHISCSELMTNDLEDDIKCAKKIARDAHGLTPWYGWKNHCEGRDLSSYVKGC

SEQ ID NO: 5 - protein, 126 aa, Opisthocomus hoazin; identical to SEQ ID NO: 4
  mat_peptide (1)..(126)

SEQ ID NO: 6 - protein, 128 aa, Artificial
  Synthetic Construct
EKRIIPRCELVKILREHGFEGFEGTTIADWICLVQHESDYNTEAYNNNGPSRDYGIFQINSKYWCNDGKTSGAVDGCHISCSELMTNDLEDDIKCAKKIARDAHGLTPWYGWKNHCEGRDLSSYVKGC